SEQUENCE LISTING (44 sequences)

SEQ ID NO: 1 - DNA, 20 bp, Artificial Sequence
  Actin-F
agagctacgagctgcctgac

SEQ ID NO: 2 - DNA, 20 bp, Artificial Sequence
  Actin-R
agcactgtgttggcgtacag

SEQ ID NO: 3 - DNA, 19 bp, Artificial Sequence
  MGMT-F
gctgcggttctcggaggtc

SEQ ID NO: 4 - DNA, 20 bp, Artificial Sequence
  MGMT-R
ctgccagggctgctaattgc

SEQ ID NO: 5 - DNA, 23 bp, Artificial Sequence
  MS-M-F
tttcgacgttcgtaggttttcgc

SEQ ID NO: 6 - DNA, 22 bp, Artificial Sequence
  MS-M-R
gcactcttccgaaaacgaaacg

SEQ ID NO: 7 - DNA, 30 bp, Artificial Sequence
  MS-U-F
tttgtgttttgatgttttgtaggtttttgt

SEQ ID NO: 8 - DNA, 28 bp, Artificial Sequence
  MS-U-R
aactccacactcttccaaaaacaaaaca

SEQ ID NO: 9 - DNA, 22 bp, Artificial Sequence
  PE1-F
taccaggaatgatgtggggtta

SEQ ID NO: 10 - DNA, 19 bp, Artificial Sequence
  PE1-R
ggcatcttccccttcacat

SEQ ID NO: 11 - DNA, 21 bp, Artificial Sequence
  PE2-F
tgaacatggcttttcaaagga

SEQ ID NO: 12 - DNA, 20 bp, Artificial Sequence
  PE2-R
cagcattccatggttgatgt

SEQ ID NO: 13 - DNA, 20 bp, Artificial Sequence
  PE3-F
ccatgcaggcatttctacct

SEQ ID NO: 14 - DNA, 20 bp, Artificial Sequence
  PE3-R
gagctcgcaggagcaactac

SEQ ID NO: 15 - DNA, 23 bp, Artificial Sequence
  PE+5kb-F
tgcaaaatttatttgggagaaaa

SEQ ID NO: 16 - DNA, 23 bp, Artificial Sequence
  PE+5kb-R
agctgccatatagaaaattggtc

SEQ ID NO: 17 - DNA, 23 bp, Artificial Sequence
  Digestion test primer
ccttttgataaatgctttctgga

SEQ ID NO: 18 - DNA, 21 bp, Artificial Sequence
  Anchor primer
atagcctaatctgccaaacga

SEQ ID NO: 19 - DNA, 30 bp, Artificial Sequence
  TaqMan Probe
catgtcttaagccacatacatttggttcct

SEQ ID NO: 20 - DNA, 21 bp, Artificial Sequence
  3C F1 primer
cctttcaggctttatggcacc

SEQ ID NO: 21 - DNA, 23 bp, Artificial Sequence
  3C F2 primer
ttgaatcatctctggttctgttt

SEQ ID NO: 22 - DNA, 27 bp, Artificial Sequence
  3C F3 primer
tcatcttcaaatactactatgccactt

SEQ ID NO: 23 - DNA, 21 bp, Artificial Sequence
  3C F4 primer
tcagtctttgcatcccctttt

SEQ ID NO: 24 - DNA, 21 bp, Artificial Sequence
  3C F5 primer
gccttctcacccagtacacaa

SEQ ID NO: 25 - DNA, 19 bp, Artificial Sequence
  3C F6 primer
ggctggccttgtactctct

SEQ ID NO: 26 - DNA, 23 bp, Artificial Sequence
  MGMT TSS F
gggagagagagagaaagatctcc

SEQ ID NO: 27 - DNA, 20 bp, Artificial Sequence
  MGMT TSS R
gaggcagggatgtccataaa

SEQ ID NO: 28 - DNA, 20 bp, Artificial Sequence
  P1
aaaatcacagcaccgcacag

SEQ ID NO: 29 - DNA, 20 bp, Artificial Sequence
  P2
ttgcagtctggcttcctcaa

SEQ ID NO: 30 - DNA, 20 bp, Artificial Sequence
  Del1-P1
aaaatcacagcaccgcacag

SEQ ID NO: 31 - DNA, 18 bp, Artificial Sequence
  Del1-P2
ctcgcaggagcaactact

SEQ ID NO: 32 - DNA, 20 bp, Artificial Sequence
  Del2-P1
tgacaaagaaacccatcagt

SEQ ID NO: 33 - DNA, 22 bp, Artificial Sequence
  Del2-P2
tttgacctatgggagaagtaga

SEQ ID NO: 34 - DNA, 20 bp, Artificial Sequence
  g1
gcagtggctttcgcatagtc

SEQ ID NO: 35 - DNA, 20 bp, Artificial Sequence
  g2
gacaggtgactctcgaagac

SEQ ID NO: 36 - DNA, 20 bp, Artificial Sequence
  g3
ttacaagtacttactcgcat

SEQ ID NO: 37 - DNA, 20 bp, Artificial Sequence
  T1
cagctcctagctgagtgtaa

SEQ ID NO: 38 - DNA, 20 bp, Artificial Sequence
  T2
ctaacgtgctcagaagatgg

SEQ ID NO: 39 - DNA, 20 bp, Artificial Sequence
  T3
ccgatttctaagtgcacaaa

SEQ ID NO: 40 - DNA, 20 bp, Artificial Sequence
  T4
gactccgtcgtcctgccacg

SEQ ID NO: 41 - DNA, 57 bp, Artificial Sequence
  WT
gtgggccagcagtggctttcgcatagtctgggacaggtgactctcgaagacaggtgg

SEQ ID NO: 42 - DNA, 35 bp, Artificial Sequence
  Deletion-1
gtgggccagcagtggctttcgcataaaacaggtgg

SEQ ID NO: 43 - DNA, 34 bp, Artificial Sequence
  Deletion-2
gtgggccagcagtgcctctaacttataaaggtgg

SEQ ID NO: 44 - DNA, 22 bp, Artificial Sequence
  Deletion-3
gtgggaatctcttgacaggtgg

(19) United States
(12) Patent Application Publication
(10) Pub. No.: US 2021/0060006 A1
(43) Pub. Date: Mar. 4, 2021

(54) COMPOSITIONS AND METHODS FOR TREATING GLIOBLASTOMA BY MODULATING A MGMT ENHANCER

(71) Applicants: The Trustees of Columbia University in the City of New York, New York, NY (US); Mayo Foundation for Medical Education and Research, Rochester, MN (US)

(72) Inventors: Zhiguo Zhang, Fort Lee, NJ (US); Xiaoyue Chen, Rochester, MN (US); Jann Sarkaria, Rochester, MN (US); Haiyun Gan, New York, NY (US)

(21) Appl. No.: 17/073,934

(22) Filed: Oct. 19, 2020

Related U.S. Application Data
(63) Continuation of application No. PCT/US19/28366, filed on Apr. 19, 2019.
(60) Provisional application No. 62/660,395, filed on Apr. 20, 2018.

(51) Int. Cl. A61K 31/495; A61P 35/04

(52) U.S. Cl. CPC A61K 31/495; A61K 45/06; A61P 35/04

(57) ABSTRACT

The present invention provides, inter alia, methods for treating or ameliorating the effects of a cancer in a subject, e.g., glioblastoma (GBM) in a subject that is resistant to temozolomide (TMZ), methods for delaying the emergence of TMZ resistance and/or increasing the TMZ sensitivity in a subject, and methods for reducing tumor aggressiveness in a subject. Also provided are pharmaceutical compositions and kits to implement such methods.

### CROSS REFERENCE TO RELATED APPLICATIONS

The present application is a continuation of PCT international application no. PCT/US2019/028366, filed on Apr. 19, 2019, which claims benefit of U.S. Provisional Patent Application Ser. No. 62/660,395, filed on Apr. 20, 2018 which applications are incorporated by reference herein in their entireties.

### GOVERNMENT FUNDING

This invention was made with government support under CA157489 awarded by the National Institutes of Health. The government has certain rights in the invention.

### INCORPORATION BY REFERENCE OF SEQUENCE LISTING

This application contains references to amino acids and/or nucleic acid sequences that have been filed concurrently herewith as sequence listing text file “CU18203-seq.txt”, file size of 9 KB, created on Oct. 2, 2020. The aforementioned sequence listing is hereby incorporated by reference in its entirety pursuant to 37 C.F.R. § 1.52(e)(5).

### FIELD OF INVENTION

The present invention provides, inter alia, methods for treating or ameliorating the effects of a cancer in a subject, e.g., glioblastoma (GBM) in a subject that is resistant to temozolomide (TMZ), methods for delaying the emergence of TMZ resistance and/or increasing the TMZ sensitivity in a subject, and methods for reducing tumor aggressiveness in a subject. Also provided are pharmaceutical compositions and kits to implement such methods.

### BACKGROUND OF THE INVENTION

Glioblastoma (GBM) is the most common and aggressive primary brain tumor. Despite current therapy, median survival for GBM patients is approximately 15 months (Stupp et al. 2005). Temozolomide (TMZ) has been the standard chemotherapy for newly diagnosed GBM for more than a decade (Stupp et al. 2005). However, almost all patients eventually develop resistance. Moreover, recurrent tumors in general are more aggressive than primary tumors. Therefore, there is a critical need to understand how TMZ resistance is acquired and whether there is any connection between TMZ resistance and tumor aggressiveness.

While multiple factors have been associated with TMZ resistance, expression of O₆-methylguanine-DNA-methytransferase (MGMT) remains a major cause (Kitange et al. 2009). MGMT is a DNA repair protein, which removes the cytotoxic O6-methylguanine (O₆MG) DNA lesions generated by TMZ, and high MGMT expression in cells is mechanistically linked to robust TMZ resistance. MGMT expression is regulated by the methylation of a promoter/enhancer (P/E) region, which contains a promoter and a 59 bp cis-acting enhancer element that spans the first exon-intron boundary of MGMT gene (Everhard et al. 2009). Several large cohort studies indicate an association between DNA methylation of the (P/E) region and favorable outcomes of TMZ treatment (Moradi et al. 2017; Brandes et al. 2017). This observation led to the hypothesis that MGMT inhibition may be a plausible strategy for sensitizing TMZ therapy in MGMT expressed tumors (Lavon et al. 2007; Wickstrom et al. 2015; Bobustuc et al. 2010; Taspinar et al. 2013). However, combinations of TMZ with MGMT inhibitors such as O₆-benzylguanine (O₆BG), a synthetic derivative of guanine that inactivates MGMT, resulted in enhanced hematologic toxicities, a reduced therapeutic window and no clinical benefit compared to TMZ alone (Quinn et al. 2009; Blumenthal et al. 2015; Warren et al. 2012). Moreover, there is significant discordance between promoter methylation status and MGMT protein expression in GBM with wild-type MGMT coding sequence (Park et al. 2012; Kreth et al. 2011; Hegi et al. 2005; Xie et al. 2011; Lund et al. 2017). These observations indicate that in addition to promoter methylation, other factors may regulate MGMT expression and confer TMZ resistance, and that identification of these additional mechanisms of MGMT regulation may provide a strong rationale for the development of a new class of drugs for this deadly disease.

Accordingly, there is a need for a new therapeutic strategy that can improve the result of TMZ treatment by delaying the emergence of TMZ resistance and/or increasing the TMZ sensitivity in a subject in need thereof. This invention is directed to meet these and other needs.

### SUMMARY OF THE INVENTION

Besides DNA methylation, post translational modifications on histone proteins also regulate gene expression. Distinct histone modifications are found at gene regulatory elements important for gene transcription (Bannister et al. 2011). For example, H3K4me3 is enriched at promoters of active genes, whereas H3K27me3 is enriched at promoters of repressed genes. In addition to promoters, enhancers, a DNA element that promotes the transcription of regulated genes via a long-range interaction with their cognate promoters, are also surrounded by nucleosomes with distinct modifications on histones (Banerji et al. 1981). Enhancers in general can be classified as active, primed or poised ones based on histone modifications of surrounding nucleosomes. Active enhancers are typically surrounded by nucleosomes with H3K4me1 and H3K27ac (Shlyueva et al. 2014). Primed enhancers are marked with H3K4me1 and a lack of histone acetylation, and poised enhancers are additionally marked by H3K27me3, a repressive mark (Heinz et al. 2015).

Without being bound to a particular theory, this invention relates to the identification of a novel distal enhancer, named K-M enhancer that is situated between MKI67 and MGMT promoters and is 560 kb away from the MGMT promoter. The K-M enhancer activates MGMT gene expression even in the presence of a hypermethylated promoter. Moreover, the K-M enhancer also regulates the expression of Ki67. Ki67 is a well-known proliferation mark for many tumor types including GBM. Increased Ki67 staining is associated with elevated aggressiveness of glioblastoma (Schlotter et al. 2017; Zhou et al. 2017; Kloppel et al. 2017). This invention shows that deletion of the enhancer results in reduced expression of MGMT as well as Ki67. Moreover, brain tumor cells lacking the enhancer are sensitive to TMZ and exhibit reduced growth rate. Together, these studies uncover a previously unknown mechanism regulating both TMZ resistance and proliferation of GBM cells.

Accordingly, one aspect of the present invention is a method for treating or ameliorating the effects of a cancer in a subject. This method comprises administering to the subject a therapeutically effective amount of a first agent that modulates the activity of an enhancer and a therapeutically effective amount of a second agent that is used to treat the cancer.

Another aspect of the present invention is a method for delaying the emergence of temozolomide (TMZ) resistance and/or increasing the TMZ sensitivity in a subject. This method comprises administering to the subject a therapeutically effective amount of an agent that modulates the activity of an enhancer.

Another aspect of the present invention is a method for reducing tumor aggressiveness in a subject. This method comprises administering to the subject a therapeutically effective amount of an agent that modulates the activity of an enhancer.

An additional aspect of the present invention is a pharmaceutical composition. This pharmaceutical composition comprises 1) a therapeutically effective amount of a first agent that modulates the activity of an enhancer in a subject suffering from a cancer and 2) a therapeutically effective amount of a second agent that is used to treat the cancer.

A further aspect of the present invention is a kit. This kit comprises the aforementioned pharmaceutical composition, and instructions of use.

Yet another aspect of the present invention is a method for treating or ameliorating the effects of gliobastoma (GBM) in a subject that is resistant to temozolomide (TMZ). This method comprises administering to the subject a therapeutically effective amount of a p300 HAT inhibitor or a Bromodomain inhibitor (BETi) and a therapeutically effective amount of TMZ.

Still another aspect of the present invention is a method of modulating proliferation of brain tumor cells with or without activation of a MGMT enhancer. This method comprises contacting the cells with an effective amount of a composition selected from a HAT inhibitor, a p300 inhibitor, a bromodomain inhibitor, and combinations thereof.

### BRIEF DESCRIPTION OF THE DRAWINGS

The application file contains at least one drawing executed in color. Copies of this patent application with color drawing(s) will be provided by the Office upon request and payment of the necessary fee.

FIGS. 1A-1I show that histone modifications marking enhancers are altered in a TMZ resistant PDX line.

FIG. 1A is the schematic diagram for the development of 5199 and 3080 xenograft lines from parental GBM12 cells. Mice with GBM12 flank tumors were treated with placebo or three cycles of TMZ (50 mg/kg/d for 5 days every 28 days). Tumors were dissected after reaching >1500 mm³. The xenograft subline established from placebo treated tumor was named 5199 and that from TMZ treated tumor was named 3080.

FIG. 1B shows the analysis of MGMT levels in protein lysates of 5199 and 3080 xenograft tissues by Western blotting. α-tubulin was used as a loading control.

FIG. 1C shows the analysis of MGMT promoter methylation in 5199 versus 3080 xenograft lines by MS-PCR. Universal methylated DNA was used as positive control and normal human brain DNA was used as negative control.

FIG. 1D is the aggregate plot showing the average ChIP-seq reads distribution for the histone mark, H3K4me1, surrounding the published enhancer regions.

FIG. 1E is the aggregate plot showing the average ChIP-seq reads distribution for the histone mark, H3K27ac, surrounding the published enhancer regions.

FIG. 1F is the aggregate plot showing the average ChIP-seq reads for the histone mark, H3K4me3, surrounding transcription start site (TSS).

FIG. 1G is the aggregate plot showing the average ChIP-seq reads for the histone mark, H3K36me3, across gene bodies. TTS: transcription termination site.

FIG. 1H provides heat maps showing cluster analysis, based on H3K27ac and H3K4me1 alterations, and expression change for genes closest to each genomic locus. The ratio of ChIP-seq reads density (3080 reads/5199 reads) for H3K4me1 and H3K27ac was calculated and subsequently analyzed by k-means cluster analysis. Changes in gene expression were calculated as RNA-seq reads ratio (3080 reads/5199 reads). Color scale represents decreased (green) and increased (red) signal intensity (intensity=log₂ 3080 reads/5199 reads).

FIG. 1I provides bar graphs showing Ingenuity Pathway Analysis for Group-1 genes. Pathways with a p-value <0.05 are presented.

FIGS. 2A-2D show the delineation of a putative enhancer associated with MGMT gene in 3080 xenograft subline.

FIG. 2A provides IGV snapshots showing H3K4me1, H3K27ac, H3K4me3, and H3K36me3 ChIP-seq reads density at the putative enhancer region, MGMT promoter and gene body in 5199 (TMZ-sensitive) and 3080 (TMZ resistant) xenograft lines.

FIG. 2B shows H3K4me1 (bottom left) and H3K27ac (bottom right) occupancy at the putative K-M enhancer in 5199 (parental) and 3080 (TMZ resistant) xenograft tumors were analyzed by ChIP-qPCR. Three different sets of primers (PE1-3) were designed to analyze against putative enhancer region. A set of primers amplifying 5 kb away from the putative enhancer (PE+5 kb) was used as negative control. Top: magnified view of H3K27ac ChIP-read peaks and the location of each primer set.

FIG. 2C shows the effect of each fragment on transcription of luciferase reporter gene in 5199 and 3080 cells. Fragments R1-R10 covering putative enhancer region (top) were cloned upstream of luciferase promoter (middle). Each construct were transfected into 5199 or 3080 cells along with a plasmid expressing Renilla luciferase. Firefly and Renilla luciferase activities were measured 36 h after transfection. Luciferase activity was normalized to the Renilla luciferase activity (internal control) first and subsequently normalized to an empty vector control.

FIG. 2D shows enhancer-promoter interaction analyzed by Chromatin Conformation Capture (3C) assay. Relative crosslinking frequency of each restriction fragment (F1-F6) was calculated as described in the experimental procedures and was plotted on genomic location of the 3′ cutting site of each fragment (X-axis). Values reported were derived from three biological repeats (*p<0.05, **p<0.01).

FIGS. 3A-3F show the analysis of the enhancer activity and MGMT expression from three pairs of primary and recurrent GBM patient samples.

FIG. 3A shows the relative occupancy of H3K4me1 in the K-M enhancer region in primary and matched recurrent tumors from three GBM patients was analyzed by ChIP-qPCR using primers described in FIGS. 2A-2D.

FIG. 3B shows the relative occupancy of H3K27ac in the K-M enhancer region in primary and matched recurrent tumors from three GBM patients was analyzed by ChIP-qPCR using primers described in FIGS. 2A-2D.

FIG. 3C shows the MGMT expression level in each tumor was analyzed by immunofluorescence. The MGMT signal (area×average intensity) from 100 individual nuclei was measured and one-way ANOVA was used for the calculation of significance. (****p<0.0001, ns: not significant, n=100)

FIG. 3D provides the analysis of MGMT expression in proliferating tumor cells. Multicolor immunofluorescence was performed using antibodies against CD45, Ki67, and MGMT. Actively dividing tumor cells (Ki67 staining without CD45 staining) were identified and the percentage of MGMT expressing cells within Ki67 positive and CD45 negative population were calculated. Two-tail paired student t-test was used for calculation of significance. (*p<0.05)

FIG. 3E provides representative images showing co-localization of MGMT and Ki67 in tissue sections from primary tumor obtained from patient #1.

FIG. 3F Representative images showing co-localization of MGMT and Ki67 in tissue sections from recurrent tumor obtained from patient #1.

FIGS. 4A-4G show that targeting p300 catalytic domain to the K-M enhancer increases H3K27ac at the enhancer region and elevates MGMT gene transcription.

FIG. 4A is a schematic diagram showing procedure of targeting p300 catalytic domain (p300 core) by CRISPR/dCas9 system. Experimental design involved co-transfection of dCas9^{p300 Core} (or control dCas9) vectors along with five guide RNAs (or a control empty vector) followed by brief selection of transfected cells under 5 μg/ml puromycin and subsequently analyzed by ChIP-qPCR.

FIG. 4B shows the analysis of the occupancy of Flag-tagged dCas9/dCas9^{p300 Core} protein at the K-M enhancer region by ChIP-qPCR in HEK293T cells 72 hours after transfection. An empty vector (lentiGuide puro vector) or a mixture of guide RNA constructs were transfected along with a dCas9 or dCas9^{p300 core} expression vector.

FIG. 4C Analysis of the occupancy of histone H3K27ac at the K-M enhancer region by ChIP-qPCR in HEK293T cells 72 hours after transfection. An empty vector (lentiGuide puro vector) or a mixture of guide RNA constructs were transfected along with a dCas9 or dCas9^{p300 core} expression vector.

FIG. 4D shows the MGMT expression in HEK293T cells was analyzed 72 hrs after co-transfection with either an empty vector or guide RNA construct along with a dCas9 or dCas9^{p300 core} expression vector. MGMT transcript level was first normalized to actin and subsequently calculated as fold change relative to double negative (Vector dCas9) control (*p<0.05, n=3 independent experiments).

FIG. 4E shows the analysis of the occupancy of Flag tagged dCas9/dCas9^{p300 core} protein at the K-M enhancer region by ChIP-qPCR in 5199 cells 72 hours after infection. 5199 cells were infected with a mixture of virus containing an empty vector (lentiGuide puro vector) or five guide RNA constructs and transfected with a dCas9 or dCas9^{p300 Core} construct.

FIG. 4F shows the analysis of the occupancy of histone H3K27ac at the K-M enhancer region by ChIP-qPCR in 5199 cells 72 hours after infection. 5199 cells were infected with a mixture of virus containing an empty vector (lentiGuide puro vector) or five guide RNA constructs and transfected with a dCas9 or dCas9^{p300 Core} construct.

FIG. 4G shows the MGMT expression in 5199 cells after targeting p300 core to the enhancer by CRISPR/dCas9. The experiments were performed as described from b-d (*p<0.05, n=3 independent experiments).

FIGS. 5A-5J shows that deletion of K-M enhancer reduces MGMT expression and increases sensitivity to TMZ.

FIG. 5A is an outline of deletion strategy using CRISPR/Cas9 system. Cells were infected with a mixture of two virus containing guide RNAs surrounding K-M enhancer locus. After puromycin selection, single clones were isolated and tested by PCR.

FIG. 5B shows that MGMT expression in SKMG3 parental cell, SKMG3 wild type clone and three K-M enhancer deleted clones were analyzed by quantitative RT-PCR. MGMT transcript level was first normalized to actin and subsequently calculated as fold change relative to SKMG3 parental line.

FIG. 5C shows that MGMT protein levels in SKMG3 parental cell, SKMG3 wild type clone and three K-M enhancer deleted clones were tested by Western blotting. α-tubulin was used as loading control.

FIG. 5D shows that SKMG3 parental line, wild type clone and K-M enhancer deleted clones were treated with indicated concentrations of temozolomide (0-1000 μM/L final concentration). Cell viability was determined using clonogenic assay.

FIG. 5E shows that SKMG3 parental line, wild type clone and K-M enhancer deleted clones were treated with 10 μM O6BG 1 h prior to temozolomide (0-1000 μM/L final concentration). Cell viability was determined using clonogenic assay.

FIG. 5F is a summary of TMZ IC₅₀ in SKMG3 clones with or without O6BG pre-treatment.

FIG. 5G shows that MGMT expression in SKMG3 parental cells and three Del1 deleted clones were analyzed by quantitative RT-PCR. MGMT transcript level was first normalized to actin and subsequently calculated as fold change relative to SKMG3 parental line.

FIG. 5H shows that SKMG3 parental cells and three Del1 deletedclones were treated with indicated concentrations of temozolomide (0-1000 μM/L final concentration). Cell viability was analyzed using clonogenic assay.

FIG. 5I shows that MGMT transcription levels in 3080 parental cells and 3080 Del1 deleted cells was analyzed by quantitative RT-PCR. MGMT transcript level was first normalized to actin and subsequently calculated as fold change relative to 3080 parental line.

FIG. 5J shows that 3080 parental cells and 3080 Del1 deleted cells were treated with different concentration of temozolomide (0-1000 μM/L final concentration). Cell viability was determined using neurosphere formation assay. (***p<0.001, ****p<0.0001, n=3 independent experiments)

FIGS. 6A-6E show that deletion of the K-M enhancer affects cell proliferation and Ki67 expression.

FIG. 6A is a schematic diagram showing the relative genomic location of MKI67 gene, the K-M enhancer and MGMT gene.

FIG. 6B shows that cell proliferation rate was analyzed by Incucyte. The cell confluency read out for each cell was normalized by the cell confluency acquired at the first time point.

FIG. 6C shows that MKI67 transcription levels in SKMG3 wild type clone and two enhancer deleted clones was analyzed by quantitative RT-PCR. Ki67 expression level was first normalized against actin and subsequently calculated as fold change relative to wild type clone.

FIG. 6D shows the growth rate of 3080 parental and 3080 Del1 cells injected flank tumors. Equal numbers of 3080 parental cells and 3080 Del1 cells were injected into the flank of mice (n=5/group). Tumor volume was used to represent growth rate.

FIG. 6E shows that MKI67 transcription levels in 3080 parental cells and 3080 Del1 deleted clone cells was analyzed by quantitative RT-PCR. Ki67 expression level was first normalized against actin and subsequently calculated as fold change relative to the parental line.

FIGS. 7A-7B show the average levels of H3K9me3 and H3K9ac in GBM xenografts.

FIG. 7A is an aggregate plot showing the average reads distribution for H3K9me3 ChIP-seq reads surrounding transcription start site.

FIG. 7B is an aggregate plot showing the average reads distribution for H3K9ac ChIP-seq reads surrounding transcription start site.

FIGS. 8A-8C show the reporter assay findings in 5199 and 3080 cells.

FIG. 8A is a schematic diagram showing the ten fragments (R1-R10) covering K-M enhancer region were cloned upstream of luciferase promoter.

FIG. 8B shows that effect of each fragment on transcription of luciferase was tested in 5199 cells. Firefly and Renilla luciferase activity were measured 36 h after transfection. Luciferase activity was normalized to the Renilla luciferase activity. Reported values were derived from 3 biological repeats. (*p<0.05, **p<0.01, ***p<0.001)

FIG. 8C shows that effect of each fragment on transcription of luciferase was tested in 3080 cells. Firefly and Renilla luciferase activity were measured 36 h after transfection. Luciferase activity was normalized to the Renilla luciferase activity. Reported values were derived from 3 biological repeats. (*p<0.05, **p<0.01, ***p<0.001)

FIGS. 9A-9C provide MGMT expression and enhancer activity profile for promoter hypermethylated PDX tumors.

FIG. 9A shows that MGMT transcription levels in eight MGMT promoter methylated PDX tumors were analyzed by quantitative RT-PCR. MGMT expression was normalized to actin.

FIG. 9B shows that H3K4me1 occupancy at the putative K-M enhancer region was analyzed in eight PDX tumors by ChIP-qPCR with primers described in FIGS. 2A-2D. Reported values were derived from three biological repeats. (*p<0.05, **p<0.01, ***p<0.001)

FIG. 9C shows that H3K27ac occupancy at the putative K-M enhancer region was analyzed in eight PDX tumors by ChIP-qPCR with primers described in FIGS. 2A-2D. Reported values were derived from three biological repeats. (*p<0.05, **p<0.01, ***p<0.001)

FIG. 10 shows CD45, Ki67, and MGMT triple immunofluorescence staining in primary and recurrent tumors from three GBM patients. Representative images showing CD45, MGMT and Ki67 co-localization in tissue sections from primary and recurrent tumor obtained from patients #1, #2, and #3. Multicolor immunofluorescence was performed using antibodies against CD45, MGMT and Ki67.

FIGS. 11A-11C provide PCR confirmation, Sanger sequencing and growth rate analysis for SKMG3 K-M enhancer deleted clones.

FIG. 11A is a gel image showing successful deletion of a 3.3 kb fragment in SKMG3 K-M enhancer deleted clones. One pair of primers spanning the sequence to be deleted was used for PCR assay.

FIG. 11B shows that a successful deletion of K-M enhancer in three SKMG3 clones was confirmed by Sanger sequencing. The location of the PAM sequence (underlined), the predicted cutting site (marked by arrow head) and the deletion patterns detected by Sanger sequencing of all K-M enhancer deletion clones were aligned on the top panel. The raw Sanger sequencing plot is displayed in the lower panel.

FIG. 11C provides images showing the clone size of SKMG3 parental cells, wild type clone and two deletion clones under DMSO treatment. The smaller clone size of enhancer deleted clones indicates impaired proliferation.

FIGS. 12A-12D provide PCR confirmation and MGMT expression for small deletion clones.

FIG. 12A is a gel image showing successful deletion of two 1.6 kb fragments in SKMG3 cells. One pair of primers spanning the sequence to be deleted was used for PCR assay. The regions to be deleted were indicated in top panel. Two pairs of primers, one spanning Del1 region another one spanning Del2 region, were used for PCR.

FIG. 12B shows that MGMT expression in SKMG3 parental line, wild type clone and Del2 deleted clones were analyzed by quantitative RT-PCR. MGMT expression level was normalized to actin and subsequently calculated as fold change relative to SKMG3 parental line.

FIG. 12C shows that the proliferation rate of SKMG3 parental cells and Del1 deleted SKMG3 clones were tested for proliferation using cell titer blue assay. The growth rate for each cell was normalized to Day 0 control.

FIG. 12D is a gel image showing successful deletion of Del1 region in 3080 cells. The PCR primers used in this assay was the same as described in FIGS. 11A-11C.

FIGS. 13A-13B show the genomic location of MKI67 gene and its expression in recurrent tumor from patient #1.

FIG. 13A is an IGV snapshot showing H3K4me1, H3K27ac, H3K4me3 and H3K36me3 read density on MKI67 gene, K-M enhancer and MGMT gene in 5199 and 3080 PDX tumors.

FIG. 13B shows that Ki67 expression level in each tumor was analyzed by immunofluorescence. The Ki67 signal (area×average intensity) from 100 individual nuclei was measured and one-way ANOVA analysis was used to calculate significance. (****p<0.0001, ns: not significant, n=100)

FIG. 14A shows that GBM cells (3080) treated with a p300/CBP inhibitor, CBP1, resulted in reduced levels of H3K27ac and reduced expression of MGMT.

FIG. 14B shows that GBM cells (3080) treated with a p300/CBP inhibitor, CPI-329, resulted in reduced levels of H3K27ac and reduced expression of MGMT.

FIG. 15A shows the result of RT-PCR analysis on the expression of MGMT in GBM cells treated with different concentrations of CBP1.

FIG. 15B shows the result of RT-PCR analysis on the expression of MGMT in GBM cells treated with different concentrations of CPI-329.

FIGS. 16A-16B show that p300 inhibition sensitized GBM cells to TMZ.

FIG. 16A shows the results of neurosphere formation assay (left) and colony formation assay (right), indicating that GBM cells treated with CBP1 resulted in increased sensitivity to TMZ.

FIG. 16B shows the results of neurosphere formation assay (left) and colony formation assay (right), indicating that GBM cells treated with CPI-329 resulted in increased sensitivity to TMZ.

### DETAILED DESCRIPTION OF THE INVENTION

Temozolomide (TMZ) has been used for the treatment of glioblastoma (GBM) since last decade, but its treatment benefits are limited by acquired resistance, a process that remains incompletely understood. In the present invention, it is shown that a novel enhancer, located between the promoters of Ki67 and O₆-methylguanine-DNA-methyltransferase (MGMT) genes, is activated in TMZ resistant patient derived xenograft (PDX) lines as well as in recurrent tumor samples. Activation of the enhancer correlates with increased MGMT expression, a major known mechanism for TMZ resistance. It is also shown that forced activation of the enhancer in cell lines with low MGMT expression results in elevated MGMT expression. Deletion of this enhancer in cell lines with high MGMT expression leads to reduced levels of MGMT and Ki67, increased TMZ sensitivity and impaired proliferation. Together, the present invention is directed to a novel mechanism that regulates MGMT expression, confers TMZ resistance and potentially regulates tumor proliferation.

Accordingly, one aspect of the present invention is a method for treating or ameliorating the effects of a cancer in a subject. This method comprises administering to the subject a therapeutically effective amount of a first agent that modulates the activity of an enhancer and a therapeutically effective amount of a second agent that is used to treat the cancer.

In some embodiment of this aspect, the enhancer regulates the expression of O₆-methylguanine-DNA-methyltransferase (MGMT) and/or Ki67 in the subject. In certain embodiments, the enhancer is K-M enhancer that is situated between MKI67 and MGMT promoters and is 560 kb away from the MGMT promoter.

In some embodiment of this aspect, the first agent is selected from a Bromodomain inhibitor (BETi), a histone acetyltransferase inhibitor (HATi), and combinations thereof. Non-limiting examples of Bromodomain inhibitors include JQ1, I-BET151/762, PF-1, RVX-208, BMS-986158, OTX015, PLX-51107, GSK525762, INCB054329, TEN-010, GSK2820151, BAY 1238097, and combinations thereof. Non-limiting examples of histone acetyltransferase inhibitors include C646, NU-9056, PU141, EML425, L002, MB-3, CPTH2, Plumbagin, Embelin, EGCG, Curcumin, HAT inhibitor II, Garcinol, Anacardic acid, MG 149, Gossypol, CTK7A, Windorphen, LoCAM, TH1834, CTx-1, Lys-CoA, and combinations thereof. In certain embodiments, the histone acetyltransferase inhibitor (HATi) is a p300 HAT inhibitor. Non-limiting examples of p300 HAT inhibitors include curcumin (CAS #458-37-7), garcinol (CAS #78824-30-3), anacardic acid (CAS #16611-84-0), C646 (CAS #328968-36-1), Demethoxy curcumin (CAS #22608-11-3), 5-Chloro-2-(4-nitrophenyl)-3(2H)-isothiazolone (CAS #748777-47-1), Histone Acetyltransferase Inhibitor II (CAS #932749-62-7), L002 (CAS #321695-57-2), CBP1, CPI-329. In some embodiments, the p300 HAT inhibitor is selected from CBP1, CPI-329, and combinations thereof.

In some embodiments of this aspect, the second agent is temozolomide (TMZ).

In some embodiments of this aspect, the subject is TMZ-resistant.

In some embodiments of this aspect, the cancer is selected from breast cancer, lung cancer, and brain tumor. In certain embodiments, the brain tumor is gliobastoma (GBM).

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, a “subject” is a mammal, preferably, a human.

In some embodiments of this aspect, this method further comprises administering to the subject a therapeutically effective amount of a histone deaceylase inhibitor (HDACi). Non-limiting examples of histone deaceylase inhibitors include Trichostatin A, SAHA, Belinostat, Panabiostat, Givinostat, Resminostat, Abexinostat, Quisinostat, Rocilinostat, Practinostat, CHR-3996, Valproic acid, Butyric acid, Phenylbutyric acid, Entinostat, Tacedinaline, 4SC202, Mocetinostat, Rom idepsin, Nicotinamide, Sirtinol, Cambinol, EX-527, and combinations thereof.

Another aspect of the present invention is a method for delaying the emergence of temozolomide (TMZ) resistance and/or increasing the TMZ sensitivity in a subject. This method comprises administering to the subject a therapeutically effective amount of an agent that modulates the activity of an enhancer.

In some embodiments of this aspect, the enhancer regulates the expression of O₆-methylguanine-DNA-methyltransferase (MGMT) in the subject. In certain embodiments, the enhancer is K-M enhancer that is situated between MKI67 and MGMT promoters and is 560 kb away from the MGMT promoter.

In some embodiment of this aspect, the agent is selected from a Bromodomain inhibitor (BETi), a histone acetyltransferase inhibitor (HATi), and combinations thereof. Non-limiting examples of Bromodomain inhibitors include JQ1, I-BET151/762, PF-1, RVX-208, BMS-986158, OTX015, PLX-51107, GSK525762, INCB054329, TEN-010, GSK2820151, BAY 1238097, and combinations thereof. Non-limiting examples of histone acetyltransferase inhibitors include C646, NU-9056, PU141, EML425, L002, MB-3, CPTH2, Plumbagin, Embelin, EGCG, Curcumin, HAT inhibitor II, Garcinol, Anacardic acid, MG 149, Gossypol, CTK7A, Windorphen, LoCAM, TH1834, CTx-1, Lys-CoA, and combinations thereof. In certain embodiments, the histone acetyltransferase inhibitor (HATi) is a p300 HAT inhibitor.

Another aspect of the present invention is a method for reducing tumor aggressiveness in a subject. This method comprises administering to the subject a therapeutically effective amount of an agent that modulates the activity of an enhancer.

In some embodiments of this aspect, the enhancer regulates the expression of Ki67 in the subject. In certain embodiments, the enhancer is K-M enhancer that is situated between MKI67 and MGMT promoters and is 560 kb away from the MGMT promoter.

In some embodiment of this aspect, the agent is selected from a Bromodomain inhibitor (BETi), a histone acetyltransferase inhibitor (HATi), and combinations thereof. Non-limiting examples of Bromodomain inhibitors include JQ1, I-BET151/762, PF-1, RVX-208, BMS-986158, OTX015, PLX-51107, GSK525762, INCB054329, TEN-010, GSK2820151, BAY 1238097, and combinations thereof. Non-limiting examples of histone acetyltransferase inhibitors include C646, NU-9056, PU141, EML425, L002, MB-3, CPTH2, Plumbagin, Embelin, EGCG, Curcumin, HAT inhibitor II, Garcinol, Anacardic acid, MG 149, Gossypol, CTK7A, Windorphen, LoCAM, TH1834, CTx-1, Lys-CoA, and combinations thereof. In certain embodiments, the histone acetyltransferase inhibitor (HATi) is a p300 HAT inhibitor.

Yet another aspect of the present invention is a pharmaceutical composition. This pharmaceutical composition comprises 1) a therapeutically effective amount of a first agent that modulates the activity of an enhancer in a subject suffering from a cancer and 2) a therapeutically effective amount of a second agent that is used to treat the cancer.

In some embodiment of this aspect, the enhancer regulates the expression of O₆-methylguanine-DNA-methyltransferase (MGMT) and/or Ki67 in the subject. In certain embodiments, the enhancer is K-M enhancer that is situated between MKI67 and MGMT promoters and is 560 kb away from the MGMT promoter.

In some embodiment of this aspect, the first agent is selected from a Bromodomain inhibitor (BETi), a histone acetyltransferase inhibitor (HATi), and combinations thereof. Non-limiting examples of Bromodomain inhibitors include JQ1, I-BET151/762, PF-1, RVX-208, BMS-986158, OTX015, PLX-51107, GSK525762, INCB054329, TEN-010, GSK2820151, BAY 1238097, and combinations thereof. Non-limiting examples of histone acetyltransferase inhibitors include C646, NU-9056, PU141, EML425, L002, MB-3, CPTH2, Plumbagin, Embelin, EGCG, Curcumin, HAT inhibitor II, Garcinol, Anacardic acid, MG 149, Gossypol, CTK7A, Windorphen, LoCAM, TH1834, CTx-1, Lys-CoA, and combinations thereof. In certain embodiments, the histone acetyltransferase inhibitor (HATi) is a p300 HAT inhibitor.

In some embodiments of this aspect, the second agent is temozolomide (TMZ).

In some embodiments of this aspect, the subject is TMZ-resistant.

In some embodiments of this aspect, the cancer is selected from breast cancer, lung cancer, and brain tumor. In certain embodiments, the brain tumor is gliobastoma (GBM).

In some embodiments of this aspect, this method further comprises administering to the subject a therapeutically effective amount of a histone deaceylase inhibitor (HDACi). Non-limiting examples of histone deaceylase inhibitors include Trichostatin A, SAHA, Belinostat, Panabiostat, Givinostat, Resminostat, Abexinostat, Quisinostat, Rocilinostat, Practinostat, CHR-3996, Valproic acid, Butyric acid, Phenylbutyric acid, Entinostat, Tacedinaline, 4SC202, Mocetinostat, Rom idepsin, Nicotinamide, Sirtinol, Cambinol, EX-527, and combinations thereof.

A further aspect of the present invention is a kit. This kit comprises the aforementioned pharmaceutical composition, and instructions of use.

Still another aspect of the present invention is a method for treating or ameliorating the effects of gliobastoma (GBM) in a subject that is resistant to temozolomide (TMZ). This method comprises administering to the subject a therapeutically effective amount of a p300 HAT inhibitor or a Bromodomain inhibitor (BETi) and a therapeutically effective amount of TMZ.

An additional aspect of the present invention is a method of modulating proliferation of brain tumor cells with or without activation of a MGMT enhancer. This method comprises contacting the cells with an effective amount of a composition selected from a HAT inhibitor, a p300 inhibitor, a bromodomain inhibitor, and combinations thereof.

The invention is further illustrated by the following examples, which are offered for illustrative purposes, and are not intended to limit the invention in any manner. Those of skill in the art will readily recognize a variety of noncritical parameters, which can be changed or modified to yield essentially the same results.

### EXAMPLES

### Example 1

### Methods and Materials

### Cell Culture

GBM cell line SKMG3 (provided by Dr. David James) and HEK293T cell line procured from ATCC were maintained in DMEM (CORNING, 10-013-CV) supplemented with 10% fetal bovine serum (Millipore Sigma, TMS-013-B) and 1% Penicillin-Streptomycin (CORNING, 30-001-CI).

GBM xenograft sublines GBM12 5199 and GBM12 3080 were developed from GBM12 patient derived xenograft and were propagated in the form of subcutaneous xenografts in athymic nude mice as previously described 26; primary cells from xenograft tissues were cultured in StemPro NSC media and supplements (ThermoFisher, A1050901) as previously described (Kitange et al. 2012).

### Xenograft Tumors

Frozen tumor tissues from xenografts established from primary GBM (GBM43, GBM59, GBM61, GBM115 and GBM122) and those from recurrent GBM (G46, G64 and G102) were developed in our lab as previously described (Kitange et al. 2017).

### Paired Patient Samples for ChIP-qPCR and Immunofluorescence Analysis

Studies involving patient samples were approved by the Mayo Clinic Institutional Review Board (IRB number 09-003015). Paired (primary and recurrent) GBM frozen tissue samples collected from consented patients, who had hypermethylated MGMT promoter at primary diagnoses and received standard TMZ treatment, were obtained from Mayo Clinic Neuro-Oncology tissue bank. A total of 3-pairs of frozen tumors with high tumor cellularity (>80%), large sample size (>30 mg) and hypermethylated MGMT promoter were selected. Tissues were cryosectioned and subjected to concomitant immunofluorescence and ChIP-qPCR analyses. Tumor samples for ChIP-qPCR were collected by scratching the tumor dense areas from the tissue sections, taking corresponding H&E stained slides as reference.

### Antibodies

Antibodies against MGMT (R&D, AF3794) and anti-α-Tubulin (Developmental Studies Hybridoma bank, 12G10) were used in western blotting analysis.

Antibodies against H3K4me1 (Abcam, Ab8895), H3K27ac (Abcam, Ab4729), H3K9ac (Abcam, Ab4441), H3K4me3 (Abcam, Ab8580), H3K9me3 (Active Motif, 39161), H3K36me3 (Active Motif, Cat #61101) and Flag (Sigma-Aldrich, 11583816001) were used for chromatin immunoprecipitation assay.

Monoclonal antibodies against CD45 (Cell Signaling, Cat #13917), MGMT (Millipore, Cat #MAB16200), Ki67 (Thermo Fisher, Cat #14-5698-82) and conjugated Alexa Fluor 488 labeled goat anti rabbit IgG (Jackson Immuno Research, Cat #111-545-144), Alexa Fluor 594 labeled goat anti mouse IgG (Life Technologies, Cat #A11032) and Cy5 labeled goat anti rat IgG (Jackson Immuno Research, Cat #112-175-167) were used for immunofluorescence.

### Western Blot Assay

Western blots were performed as previously described (Fang et al. 2016). Primary antibodies were used under 1:1000 dilution.

### RNA Isolation Reverse Transcription and Real-Time PCR

RNA was extracted with RNeasy plus kit (Qiagen, #74134) according to the manufacturer's instructions. Reverse transcription of mRNA was performed using SuperScript™ III Reverse Transcriptase (Invitrogen, 18080-085). For real time PCR analysis, 1 μl of cDNA (25 ng of starting RNA) was amplified per reaction using the iTaq Universal SYBR Green Supermix (Bio-Rad, 172-5124) and the Bio-Rad CFX qPCR system as previously described (Fang et al. 2016). Primers for real time qPCR analysis were listed in Table 1.

### RNA-Seq

RNA sequencing was performed as previously described (Kitange et al. 2016). Briefly, total RNA was extracted as described above. The RNA quality was further evaluated using the Agilent 2100 Bioanalyzer (Agilent, Santa Clara, Calif.). The Illumina TrueSeq RNA Sample preparation Kit v.4.1 (Illumina Inc., San Diego, Calif.) was used to prepare cDNA libraries from 2 μg of total RNA for RNA-seq. Individual barcoded libraries were analyzed using Agilent 2100 Bioanalyzer (Agilent technologies). Sequencing was carried out on an Illumina HiSeq 2000 machine (Illumina) at Mayo Clinic Medical Genomic Facility.

### Ms-PCR

DNA was extracted from frozen tissues or cells using Blood & Cell Culture DNA Mini Kit (Qiagen, #13323). Isolated genomic DNA was bisulfite-treated with EZ DNA methylation Gold kit (Zymo Research, #11-335B). The modified DNA was quantified by PCR. MS-M-F/R primers were used to PCR methylated MGMT promoter, MS-U-F/R primers were used to PCR unmethylated MGMT promoter. The sequences for PCR primers were listed in Table 1.

### Chromatin Immunoprecipitation

Chromatin immunoprecipitation assay was performed as described (Fang et al. 2016). 1×10⁶ cells or 15 mg homogenized frozen tissues were fixed with 1% paraformaldehyde at room temperature for 10 min, quenched with 0.125 M glycine and lysed for 10 min on ice. The lysate was digested with MNase (NEB, Cat #M0247S) at 2000 gel unit/ml final concentration at 37° C. for 20 min and sonicated 15 cycles (30 s on, 30 s off) under high power using a Diagenode Bioruptor. Crosslinked DNA was immunoprecipitated with 2 μg antibody at 4° C. overnight, pulled down by protein G beads, washed, reverse crosslinked and purified for qPCR for high throughput sequencing analysis.

For ChIP-qPCR analysis, both input and immunoprecipitated DNA were quantified by real-time PCR with primers listed in Table 1. DNA quantity for each ChIP sample was normalized against input DNA.

For ChIP-seq samples, after DNA purification ChIP-seq DNA libraries were prepared with the Ovation Ultralow DR Multiplex system (NuGEN). The DNA libraries were sequenced using the 51 bp paired-end sequencing method by an IIlumina Hi-seq 2000.

### ChIP-Seq Analysis

Raw reads from IIlumina Hi-seq 2000 were aligned to the human genome (hg19) using Bowtie2 software with default parameters. Only uniquely mapping reads were used for secondary analysis. The ChIP-seq peaks were identified by MACS2 using the default calling parameter; and the p value of cutoff was set to 0.001. Genome-wide read coverage was calculated by BEDTools and in-house Perl programs, and visualized using Integrative Genomics Viewer as previously described (Zhang et al. 2017). The reads density scan was performed by in-house Perl programs using the traditional normalization method: Reads per Kilobase per Million Mapped Reads (RPKM).

For cluster analysis, firstly genomic regions with high H3K4me1 and low H3K4me3 occupancy in 5199 and 3080 cells were selected. H3K4me1 peaks were merged if their distance is less than 500 bp. The

$${log}_{2} \frac{H 3 K 4 me 1 ( 3080 )}{H 3 K 4 me 1 ( 5199 )} and {log}_{2} \frac{H 3 K 27 ac ( 3080 )}{H 3 K 27 ac ( 5199 )}$$

on those merged peaks were used for unsupervised k-means cluster analysis.

### Pathway Analysis

The Group-1 genes identified from cluster analysis were imported into Ingenuity pathway analysis (IPA) program. A list of affected pathways was calculated based on default setting.

### Reporter Assay

DNA fragments tested in a reporter assay are named as reporter fragments (R1 to R10). Those fragments were inserted upstream of SV40 promoter and Firefly luciferase in a pGL3 promoter vector. For each transfection reaction, 100 ng control plasmid expressing Renilla luciferase and 300 ng Firefly luciferase construct were co-transfected into 2×10⁵ cells in a 24 well plate well. After 24 h, luciferase activities were measured by the Dual-Luciferase Reporter Assay System (Promega, E1910).

### Chromatin Conformation Capture Assay

The chromatin conformation capture assay was performed as described (Hagege et al. 2007). Briefly, cells were crosslinked with 1% formaldehyde at room temperature for 10 min, quenched with 0.125 M glycine, lysed, and treated with 600 U HindIII (NEB, #R3104) at 37° C. overnight followed by a 4 h ligation with T4 enzyme (NEB, M0202L) at 16° C. Ligated products were quantified in triplicate by TaqMan real-time PCR. Probes and primers (listed in Table 1) were designed by using primer blast provided by NCBI. Control 3C template was generated by using two bacterial artificial chromosomes (BACs), 656G14 and 1125P18, which together encompass putative K-M enhancer and MGMT promoter regions. Equimolar of the two BACs were digested with HindIII and ligated. The ligation product from BAC control was used for normalization. The relative interaction frequency was calculated as: 2^{Ct(BAC)-Ct(3C)}

### Immunofluorescence

OCT embedded patient tumor tissues were sectioned at 5 micron thickness. Slides were fixed by 4% paraformaldehyde at 4° C. for 10 minutes, penetrated with PBS plus 0.25% Triton X-100 for 10 min and pretreated with steam TBS antigen retrieval buffer at pH 9.0 for 60 minutes. One hour primary antibody incubation (1:100 dilution) was performed at room temperature followed by one hour secondary antibody incubation (1:200 dilution). Slides were rinsed, dehydrated and mounted with Prolong Gold antifade mounting media with DAPI (Invitrogen, Cat #P36935) and analyzed by confocal microscopy (LSM 780; 63× objective lenses). MGMT and Ki67 were considered positive when uniform staining was detected in cell nuclei. CD45 was considered positive when cytoplasmic staining was detected.

The signal intensity for each cell was quantified in ImageJ program. The area of each nucleus was determined by DAPI staining. The total signal for each nucleus was calculated as: signal=area×average signal intensity. The total signal for 100 nuclei from each slides were quantified and plotted.

The investigator was blinded to sample allocation during immunofluorescence image collection and counting.

### Guide RNA Design and Cloning

All guide RNAs were designed by using MIT CRISPR Design website (http://crispr.mit.edu). To minimize potential off-target effects of guide RNA, only high score guide RNAs (score >85) were used. Guide RNA sequences are listed in Table 2.

Guide RNAs used in CRISPR/dCas9 system were cloned into lentiGuide puro vector (Addgene, Plasmid #52963) by using a published protocol (Sanjana et al. 2014). Guide RNAs used in CRISPR/Cas9 were cloned into lentiCRISPRv2 vector using the same protocol.

### Enhancer Activation by CRISPR/dCas9^{p300 Core} System

Guide RNAs were cloned into lentiGuide puro vector (Addgene, Plasmid #52963). 5199 cells were infected with a mixture of virus containing five guide RNAs while HEK293T cells were co-transfected with a pooled guide RNA containing five guide RNA constructs. Both 5199 cell and HEK293T cells are transfected with a pcDNA-dCas9-p300 (or control dCas9) Core plasmid (Addgene, Plasmid #61357). Puromycin selection was performed 24 h post transfection. Targeting of dCas9^{p300 Core} protein was confirmed by Flag ChIP-qPCR assay at 72 h post transfection. Enhancer activity and MGMT transcript were assessed by H3K27ac ChIP-qPCR and RT-PCR respectively. Primers used for ChIP-qPCR and RT-PCR assay are listed in Table 1.

### CRISPR/Cas9-Mediated Genomic Deletion

Guide RNAs were cloned into lentiCRISPR v2 vector (Addgene, Plasmid #52961). Lentivirus for guide RNAs were produced as previously described (Zhang et al. 2017). For genomic deletion model, SKMG3 cells were infected with equal amount of two lentivirus carrying guide RNAs flanking the region to be deleted, followed by clonal selection under puromycin and clone expansion. Paired guide RNAs g1/g2 were used to generate larger deletion, while paired guide RNAs g1/g3 and g2/g3 were used to generate the first smaller deletion and the second smaller deletion. PCR amplification was used for genotypic characterization of putative deletion clones. The sequence of those primers is listed in Table 1. The PCR products of positive clones with homozygous deletion were validated by Sanger sequencing.

### Clonogenic Assay

Clonogenic assays were performed to assess the effect of disrupted enhancer on TMZ sensitivity. Briefly, SKMG3 parental cells and enhancer deletion clones were plated in 6 wells plates (250 cells/well), treated with graded concentration of TMZ in presence or absence of O6BG and were cultured for 2 weeks. Colonies were fixed and stained with crystal violet (0.005% (w/v) Crystal violet, 25% (v/v) Methanol). Colonies with >50 cells were manually counted, IC₅₀ calculated by GraphPad Prism 7 using multiparametric nonlinear regression model.

### Neurosphere Assay

Neurosphere assay was performed as previously described (Kitange et al. 2012). In brief, primary cells suspended in StemPro NSC media were plated in triplicate in 96-well plates (500 cells per well) and treated with graded concentration of TMZ (0-1000 μM/L final concentration). Intact neurospheres containing more than 50 cells were counted after 15 days. Cell viability was calculated relative to DMSO control. IC₅₀ was calculated by GraphPad Prism 7 7 using multiparametric nonlinear regression model.

### Cell Proliferation Assay

IncuCyte machine was used to measure cell proliferation rate of SKMG3 wild type clone and K-M enhancer deleted clones. Cells were plated in 96 well plates in triplicate at 500 cells per well density. The percentage of confluency for each well was measured for 6 days. The confluency data was used to calculate proliferation rate as fold changes compare to Day 0.

CellTiter-Blue® Cell Viability Assay kit (CTB) was used to measure cell viability for SKMG3 and SKMG3-Del1 clones. Cells were plated in 96-well plates in triplicate at 1,000 cells per well density. Cell number was measured every other day according to kit manufacturer's instruction. Fluorescence signal was read by GloMax®-Muti Microplate Multimode Reader with excitation at 560 nm and emission at 590 nm.

### Flank Tumor Injection

1×10⁶ cells were injected into the flank of athymic mice. Mice were euthanized and tumors harvested when the tumor size exceeded 2,000 mm³.

### Statistical Analysis

ChIP-qPCR analysis for patient samples was not replicated due to limited sample size. All other experiments were done with three biological replicates.

A two-tailed Student's t test was used to establish statistical significance between control and testing group for all comparison between two data sets. One way ANOVA was used for MGMT and Ki67 signal intensity comparison analysis.

TABLE 1
Primers and TaqMan probe sequences
used for MS-PCR and qPCR.

| Primer name | Sequence(5′-3′) | Assay |
|---|---|---|
| Actin-F | AGAGCTACGAGCTGCC | RT-PCR |
| | TGAC | |
| | (SEQ ID NO: 1) | |
| Actin-R | AGCACTGTGTTGGCG | RT-PCR |
| | TACAG | |
| | (SEQ ID NO: 2) | |
| MGMT-F | GCTGCGGTTCTCGGAG | RT-PCR |
| | GTC | |
| | (SEQ ID NO: 3) | |
| MGMT-R | CTGCCAGGGCTGCTAA | RT-PCR |
| | TTGC | |
| | (SEQ ID NO: 4) | |
| MS-M-F | TTTCGACGTTCGTAG | MS-PCR |
| | GTTTTCGC | |
| | (SEQ ID NO: 5) | |
| MS-M-R | GCACTCTTCCGAAAAC | MS-PCR |
| | GAAACG | |
| | (SEQ ID NO: 6) | |
| MS-U-F | TTTGTGTTTTGATGTT | MS-PCR |
| | TTGTAGGTTTTTGT | |
| | (SEQ ID NO: 7) | |
| MS-U-R | AACTCCACACTCTT | MS-PCR |
| | CCAAAAACAAAACA | |
| | (SEQ ID NO: 8) | |
| PE1-F | TACCAGGAATGATGTGG | ChIP-qPCR |
| | GGTTA | |
| | (SEQ ID NO: 9) | |
| PE1-R | GGCATCTTCCCCTTCA | ChIP-qPCR |
| | CAT | |
| | (SEQ ID NO: 10) | |
| PE2-F | TGAACATGGCTTTTCA | ChIP-qPCR |
| | AAGGA | |
| | (SEQ ID NO: 11) | |
| PE2-R | CAGCATTCCATGGTTG | ChIP-qPCR |
| | ATGT | |
| | (SEQ ID NO: 12) | |
| PE3-F | CCATGCAGGCATTTCT | ChIP-qPCR |
| | ACCT | |
| | (SEQ ID NO: 13) | |
| PE3-R | GAGCTCGCAGGAGCAA | ChIP-qPCR |
| | CTAC | |
| | (SEQ ID NO: 14) | |
| PE + 5kb − F | TGCAAAATTTATTTG | ChIP-qPCR |
| | GGAGAAAA | |
| | (SEQ ID NO: 15) | |
| PE + 5kb − R | AGCTGCCATATAGAA | ChIP-qPCR |
| | AATTGGTC | |
| | (SEQ ID NO: 16) | |
| Digestion | CCTTTTGATAAATGCT | 3C |
| test | TTCTGGA | |
| primer | (SEQ ID NO: 17) | |
| Anchor primer | ATAGCCTAATCTGCCA | 3C |
| | AACGA | |
| | (SEQ ID NO: 18) | |
| TaqMan Probe | FAM-CATGTCTT | 3C |
| | AAGCCACATACA | |
| | TTTGGTTCCT- | |
| | TAMRA | |
| | (SEQ ID NO: 19) | |
| 3C F1 primer | CCTTTCAGGCTTTATG | 3C |
| | GCACC | |
| | (SEQ ID NO: 20) | |
| 3C F2 primer | TTGAATCATCTCTGGT | 3C |
| | TCTGTTT | |
| | (SEQ ID NO: 21) | |
| 3C F3 primer | TCATCTTCAAATACTA | 3C |
| | CTATGCCACTT | |
| | (SEQ ID NO: 22) | |
| 3C F4 primer | TCAGTCTTTGCATCCC | 3C |
| | CTTTT | |
| | (SEQ ID NO: 23) | |
| 3C F5 primer | GCCTTCTCACCCAGT | 3C |
| | ACACAA | |
| | (SEQ ID NO: 24) | |
| 3C F6 primer | GGCTGGCCTTGTACTC | 3C |
| | TCT | |
| | (SEQ ID NO: 25) | |
| MGMT TSS F | GGGAGAGAGAGAGAAA | 3C |
| | GATCTCC | |
| | (SEQ ID NO: 26) | |
| MGMT TSS R | GAGGCAGGGATGTCCA | 3C |
| | TAAA | |
| | (SEQ ID NO: 27) | |
| P1 | AAAATCACAGCACCGC | K-M deletion |
| | ACAG | confirmation-F |
| | (SEQ ID NO: 28) | |
| P2 | TTGCAGTCTGGCTTCC | K-M deletion |
| | TCAA | confirmation-R |
| | (SEQ ID NO: 29) | |
| Del1-P1 | AAAATCACAGCACCGC | Del1 deletion |
| | ACAG | confirmation-F |
| | (SEQ ID NO: 30) | |
| Del1-P2 | CTCGCAGGAGCAACTA | Del1 deletion |
| | CT | confirmation-R |
| | (SEQ ID NO: 31) | |
| Del2-P1 | TGACAAAGAAACCCAT | Del2 deletion |
| | CAGT | confirmation-F |
| | (SEQ ID NO: 32) | |
| Del2-P2 | TTTGACCTATGGGAGA | Del2 deletion |
| | AGTAGA | confirmation-R |
| | (SEQ ID NO: 33) | |

TABLE 2
Guide RNA sequences for CRISPR-dCas9
based P300 targeting assay and CRISPR-Cas9
based genomic deletion assays.

| guide | | |
|---|---|---|
| RNA | | |
| names | Sequence | Used for |
| g1 | GCAGTGGCTTTCGCATAGTC | Deletion |
| | (SEQ ID NO: 34) | |
| g2 | GACAGGTGACTCTCGAAGAC | Deletion |
| | (SEQ ID NO: 35) | |
| g3 | TTACAAGTACTTACTCGCAT | Deletion/ |
| | (SEQ ID NO: 36) | Targeting |
| T1 | CAGCTCCTAGCTGAGTGTAA | Targeting |
| | (SEQ ID NO: 37) | |
| T2 | CTAACGTGCTCAGAAGATGG | Targeting |
| | (SEQ ID NO: 38) | |
| T3 | CCGATTTCTAAGTGCACAAA | Targeting |
| | (SEQ ID NO: 39) | |
| T4 | GACTCCGTCGTCCTGCCACG | Targeting |
| | (SEQ ID NO: 40) | |

### Example 2

### Altered Histone Modifications at Enhancers in a TMZ Resistant GBM Xenograft Line

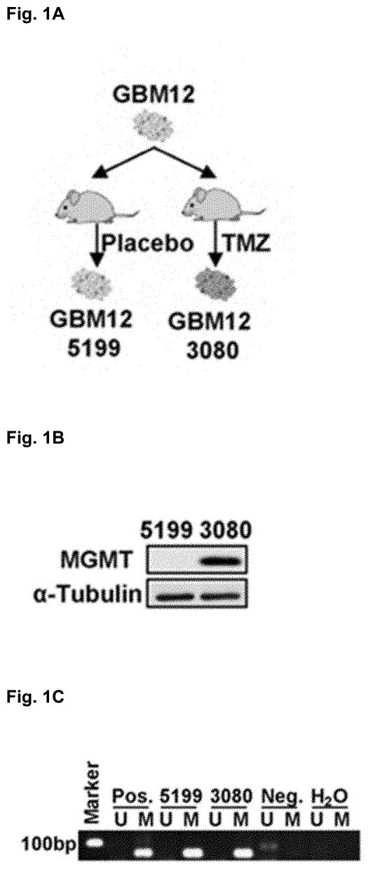

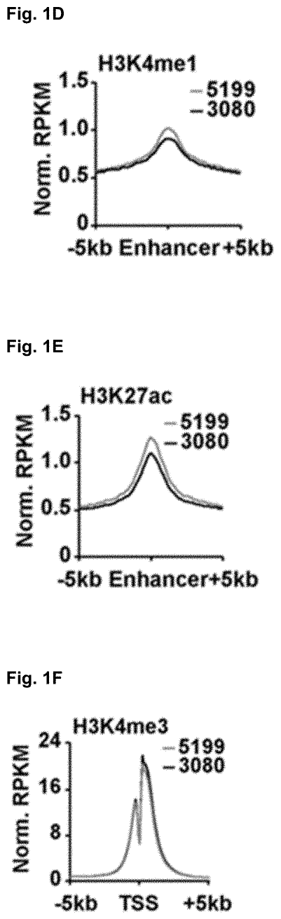

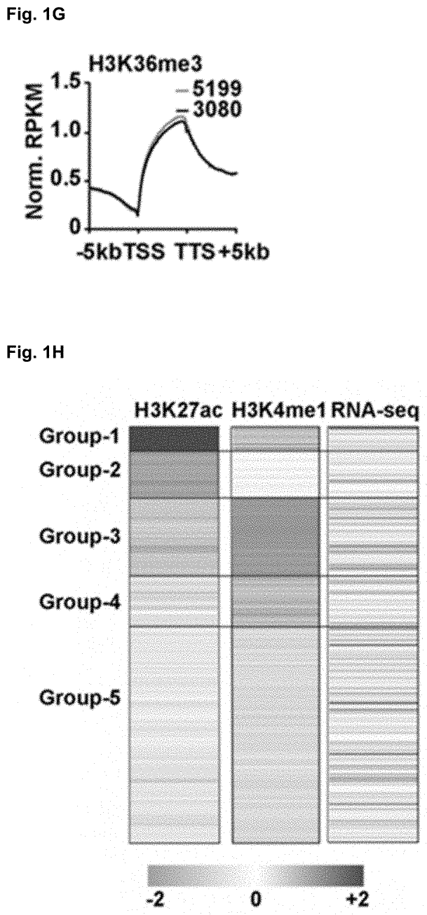

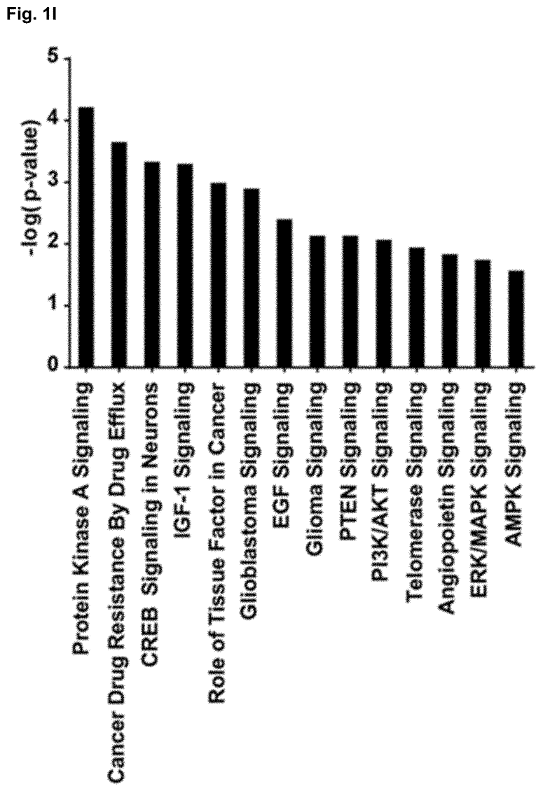

To investigate the genetic and epigenetic changes that occur during tumor recurrence, we used a patient-derived xenograft (PDX) model previously described (Kitange et al. 2012). The GBM12 xenograft line derived from a newly diagnosed MGMT hypermethylated tumor was used to generate TMZ resistant sublines. For this, multiple mice with flank tumors generated from GBM12 were treated with 3 cycles of TMZ or placebo. Two tumor sub-lines, a TMZ sensitive tumor from the placebo group named GBM12-5199 (5199) and a TMZ resistant tumor from the TMZ treatment group named GBM12-3080 (3080) were obtained (FIG. 1A). Similar to the original hypermethylated GBM12 tumor, the placebo-treated 5199 line had low MGMT protein expression and was highly susceptible to TMZ. In contrast, the TMZ-resistant 3080 line had robust MGMT expression despite of the presence of MGMT promoter methylation (FIGS. 1B-1C). These results suggest that other mechanisms were driving MGMT expression even in the presence of the MGMT promoter methylation.

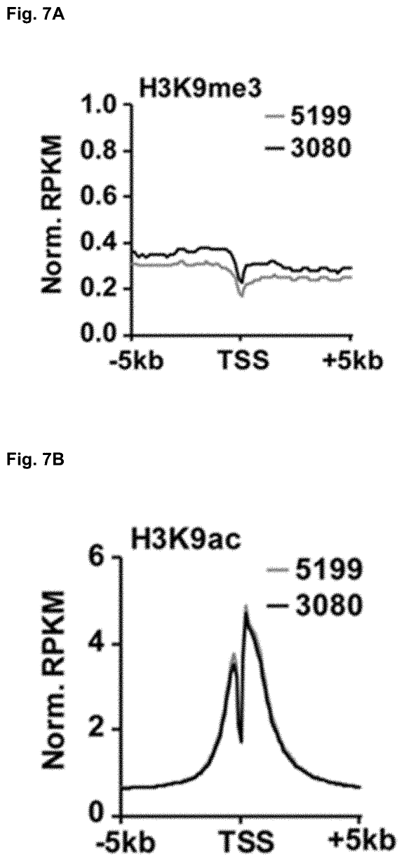

In addition to DNA methylation, histone modifications also play an important role in the regulation of gene expression (Kitange et al. 2012; Allis et al. 2016). To test whether changes in histone modifications were associated with elevated MGMT expression in 3080, we analyzed histone modifications for active enhancers (H3K4me1 and H3K27ac), promoters (H3K4me3 and H3K9ac), or gene bodies (H3K36me3) and repressed mark (H3K9me3) in 5199 and 3080 sublines using Chromatin Immunoprecipitation coupled with next-generation sequencing (ChIP-seq) (FIGS. 1D-1G and FIGS. 7A-7B). We found that the global levels of H3K4me3 and H3K9ac at promoters and H3K36me3 at gene bodies were similar between 5199 and 3080. Interestingly the levels of H3K4me1 and H3K27ac at the enhancer regions were globally reduced in the 3080 line compared to the 5199 line. Moreover, the level of H3K9me3 was increased at the transcription starting sites (TSS) in the TMZ-resistant 3080 compared to the TMZ-sensitive 5199 (FIGS. 7A-7B). We noted that a previous reports showing that H3K9me2/3 increases globally in GBM cells upon TMZ treatment (Braig et al. 2005). These results suggest that histone modifications are susceptible to alterations during the acquisition of TMZ resistance.

Enhancer activation, which is characterized by increased H3K27ac at enhancers regions, leads to elevated transcription of nearby genes. To classify enhancers, putative enhancers with at least one enhancer mark altered in the recurrent 3080 line were used for unsupervised clustering analysis based on the changes of H3K4me1 and H3K27ac levels and the correlations with the expression change of genes close to each putative enhancer (FIG. 1H). In comparison to 5199 line, we observed that most enhancers exhibited reduced levels of H3K27ac and H3K4me1 in 3080 line, consistent with our previous observation that these two marks were reduced globally in 3080 compared to 5199 line. A small group of enhancers (1141 Group-1 enhancers) exhibited increased H3K27ac and H3K4me1 in 3080 line, suggesting that this group of enhancers is activated in the TMZ resistant line. Moreover, the activation of this group of enhancers correlated with increased expression of nearby genes. Pathway analysis of this group of genes indicated that genes that are involved in gliomagenesis and cancer drug resistance are enriched in this group of genes (FIG. 1I). These results suggest that a subgroup of enhancers was activated in the 3080 line and may contribute to TMZ resistance.

### Example 3

### MGMT is Regulated by a Novel Enhancer

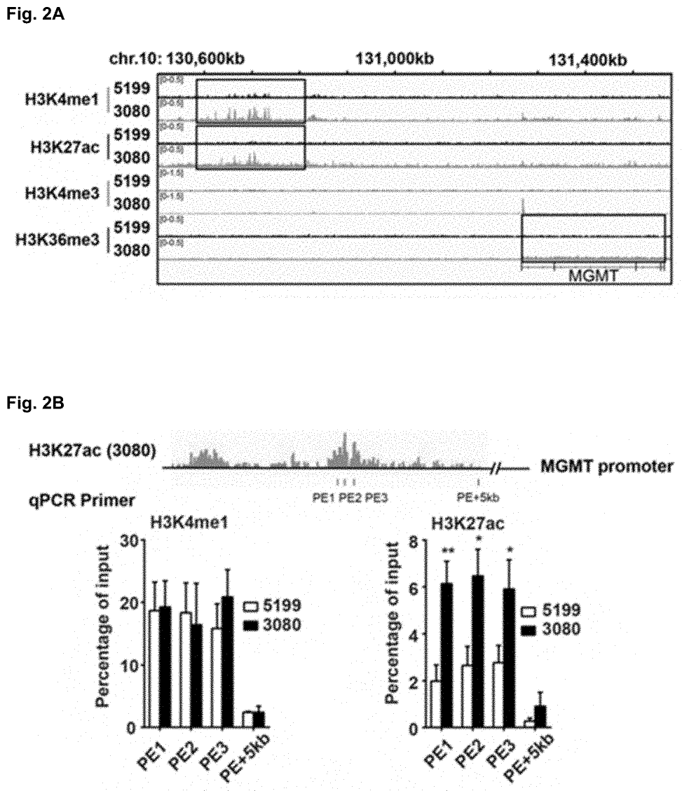

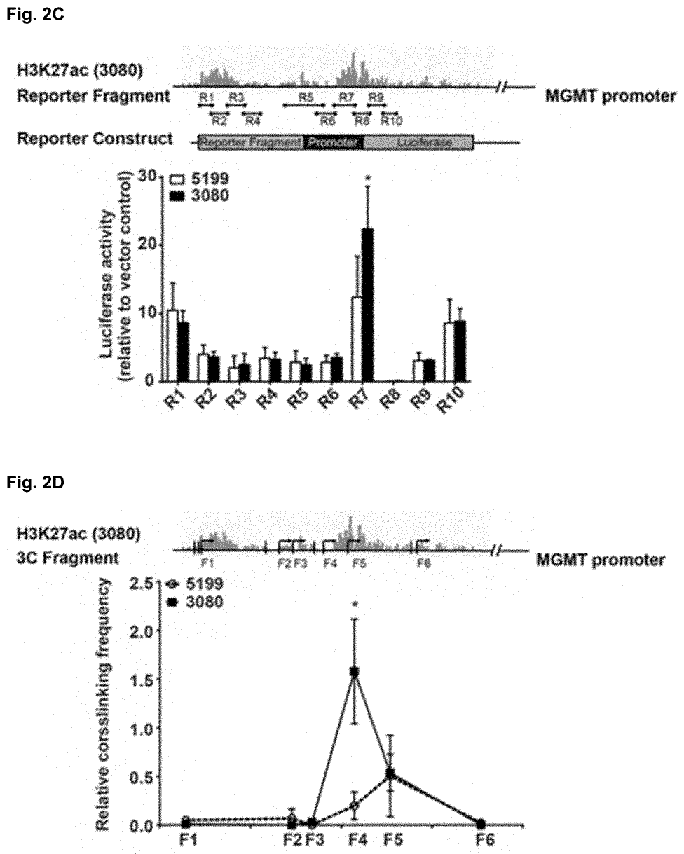

Interestingly, MGMT, a key driver of TMZ resistance (Gerson et al. 2004), was one of the top 10 genes in the Group-1 gene list (Table 3). We therefore inspected H3K4me1, H3K27ac, H3K4me3 and H3K36me3 ChIP-seq peaks close to the MGMT gene locus (FIG. 2A). In line with increased MGMT transcription, H3K36me3 within the gene body and H3K4me3 in the promoter region were enriched in the 3080 line compared to 5199 line. Moreover, we also detected an increase in H3K4me1 and H3K27ac enrichment in the 3080 line compared to 5199 line at a region 560 kb away from the MGMT promoter, suggesting that this region may be a putative enhancer that can activate MGMT expression. Because this putative enhancer was localized in an intergenic region between MKI67 gene and MGMT gene, we named this putative enhancer K-M enhancer. By analyzing immunoprecipitated chromatin DNA using four pairs of primers, including three pairs spanning the putative K-M enhancer (PE1-3) region and a control region 5 kb away from putative enhancer region (PE+5 kb), with quantitative PCR (ChIP-qPCR), we confirmed that the levels of H3K27ac was significantly higher in the 3080 line than the 5199 line (FIG. 2B), whereas an equivalent level of H3K4me1 within the putative enhancer was detected between these two lines. The discrepancy in H3K4me1 at the putative enhancer detected by ChIP-qPCR and ChIP-seq may be due to the fact that H3K4me1 can be detected in the distal region. Alternatively, this putative enhancer could be in primed state, characterized by the presence of H3K4me1 and low levels of H3K27ac in the 5199 line. Collectively, these results suggest that a putative enhancer is activated in the TMZ resistant 3080 line and potentially drives MGMT expression.

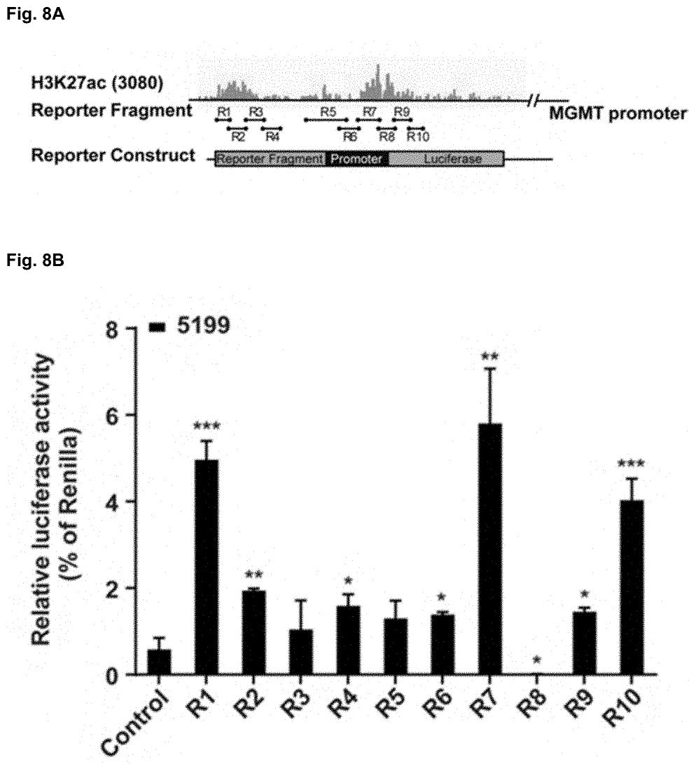

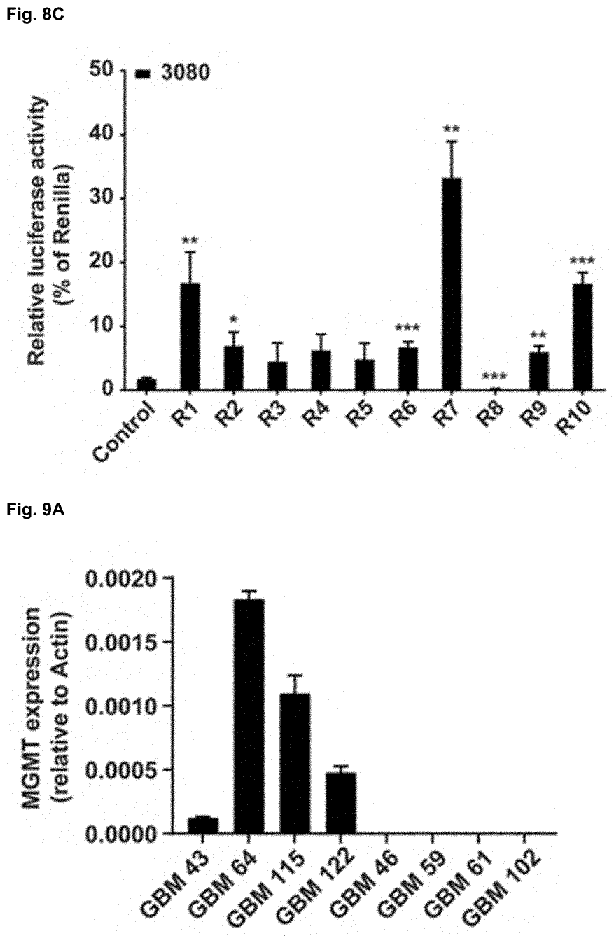

Enhancers are regulatory elements that can promote gene expression. Therefore, we first tested whether this novel enhancer region can enhance transcription of a luciferase reporter gene. We cloned 10 DNA fragments (R1 to R10), each 1-2 kb in size, spanning the 13.5 kb H3K27ac peak region, in front of an SV40 promoter-driven firefly luciferase reporter. Each reporter construct was co-transfected with a pRL Renilla luciferase control reporter construct, which constitutively expresses Renilla luciferase to allow for normalization of transfection efficiency. While R1, R2, R6, R7, R9 and R10 significantly stimulated transcription compared to pGL3 vector control (FIGS. 8A-8C), only the 1.5 kb R7 fragment, localized in the second H3K27ac peak region, exhibited significantly higher activity in the 3080 line compared to 5199 (FIG. 2C). These results suggest that this region can enhance gene transcription and the R7 region may act as an MGMT enhancer, whereas other regions may serve as enhancer for other genes such as Ki67.

Enhancers typically contact with their cognate gene promoters through long-range interactions (Sur et al. 2016; Hnisz et al. 2013; Herz et al. 2014). We next tested whether the putative enhancer interacts with the MGMT promoter using the Chromatin Conformation Capture (3C) assay (FIG. 2D). In 3080 cells, a strong interaction between K-M enhancer and MGMT promoter was identified. The fourth test fragment (F4), which overlaps with the R7 region tested in the reporter assay, displayed significantly higher interaction frequency with the MGMT promoter compared to the neighboring DNA fragments. Moreover, the interaction frequency between the F4 fragment and the MGMT promoter in the 5199 line, which lacks MGMT expression, was significantly lower than the 3080 line, supporting the idea that the putative K-M enhancer region specifically interacts with the MGMT promoter in the 3080 line. Therefore, the 1.5 kb region located 560 kb away from the MGMT promoter has characteristics of an active enhancer (surrounding by nucleosomes with high H3K27ac, transcription enhancement, and interaction with promoter). The differential activity between the placebo 5199 line and the TMZ resistant 3080 line further underscored the idea that this novel enhancer was specifically activated to stimulate MGMT transcription, even in the presence of MGMT promoter methylation in 3080 line.

TABLE 3
Top 10 most strongly altered genes in Group-1.

| Rank | Gene name | Full name |
|---|---|---|
| 1 | MGMT | O-6-Methylguanine-DNA Methyltransferase |
| 2 | ST18 | Human suppression of tumorigenicity 18 |
| 3 | WDFY4 | WD repeat-and FYVE domain-containing protein 4 |
| 4 | C17orf50 | Chromosome 17 Open Reading Frame 50 |
| 5 | WBSCR17 | Williams-Beuren Syndrome Chromosome Region 17 |
| 6 | ACTL8 | Actin Like 8 |
| 7 | MAP3K21 | Mitogen-activated protein kinase kinase kinase 21 |
| 8 | SORCS3 | Sortilin Related VPS10 Domain Containing Receptor 3 |
| 9 | BHLHA9 | Basic Helix-Loop-Helix Family Member A9 |
| 10 | TTR | Transthyretin |

Strongly elevated genes within the 1141 genes in Group-1 are listed in the table. Genes were sorted from top to bottom based on the ratio of its expression in 3080 line divided by its expression in 5199 line.

### Example 4

### The Enhancer is Active in a Fraction of PDX Lines and Primary Tumor Samples

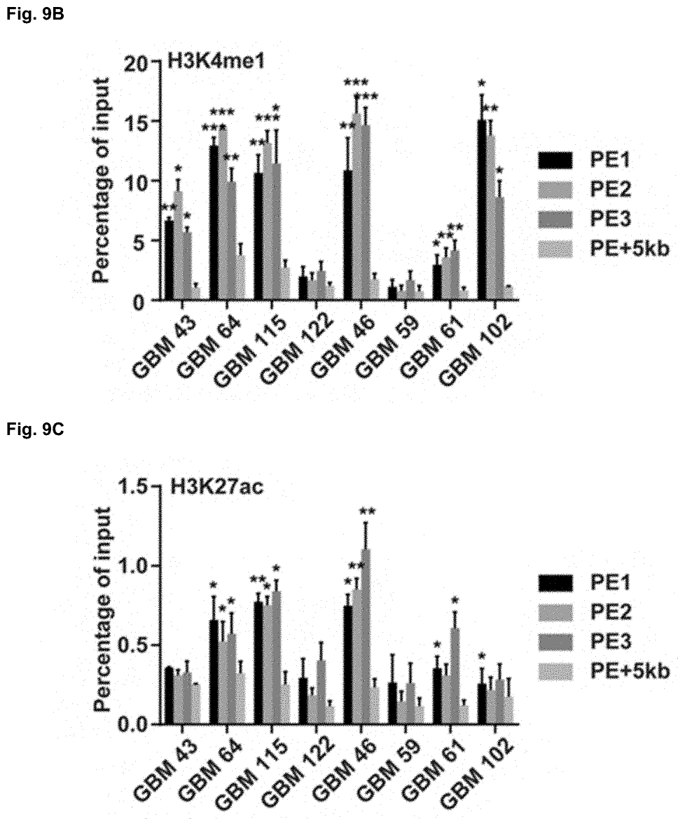

In addition to the 3080 line, we have detected MGMT expression in four MGMT promoter methylated xenograft lines previously (data not shown). To investigate whether the enhancer was activated in any of these MGMT expressing, promoter methylated PDX lines, we analyzed H3K4me1 and H3K27ac using ChIP-PCR in eight MGMT promoter methylated PDX lines including 4 PDX lines expressing MGMT (GBM43, GBM64, GBM115 and GBM122) and 4 lines without MGMT expression (GBM46, GBM59, GBM61 and GBM102) (FIG. 9A). H3K4me1 ChIP-qPCR analysis showed that 6 PDX lines (GBM43, GBM64, GBM115, GBM46, GBM61 and GBM102) had high levels of H3K4me1 at the enhancer region compared to a fragment 5 kb away (FIG. 9B). H3K27ac ChIP-qPCR analysis indicated that of the 6 samples with H3K4me1, three samples (GBM64, GBM115, and GBM46) had higher levels of H3K27ac at the K-M enhancer region compared to control locus (FIG. 9C), suggesting that the K-M enhancer is activated in these three lines. The GBM46 line is a MGMT low-expressing line from a recurrent tumor, whereas the two MGMT expressed lines, GBM115 and GBM64 are from primary and recurrent tumor, respectively, and have high levels of MGMT. Although the levels of H3K4me1 and H3K27ac enrichment at the K-M enhancer locus do not correlate with MGMT expression in GBM46 line, activation of the enhancers in two other MGMT promoter methylated PDZ lines correlates with high levels of MGMT expression, suggesting that enhancer activation is one explanation for the discordance between MGMT promoter methylation and gene expression.

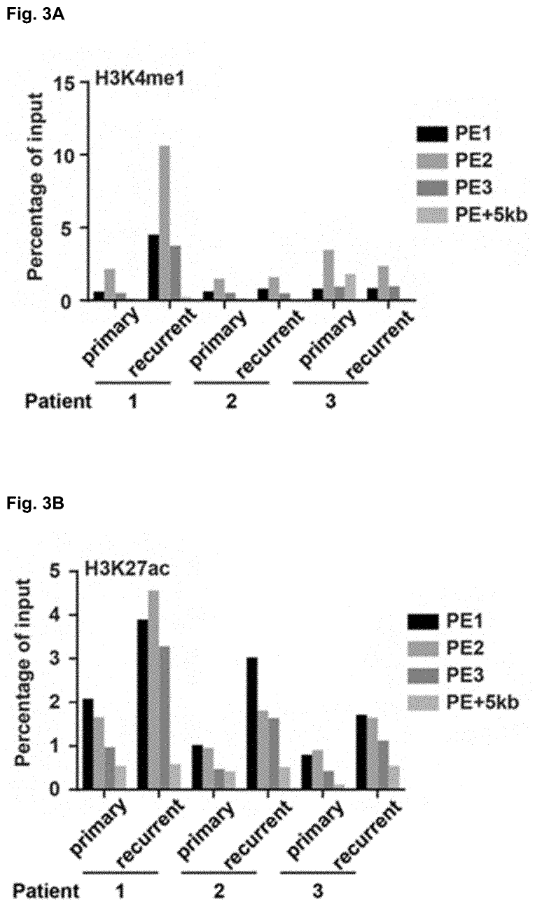

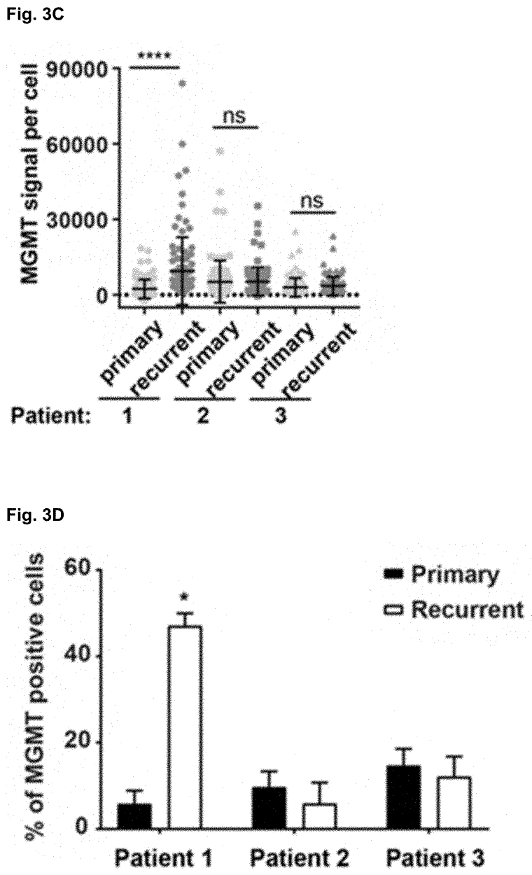

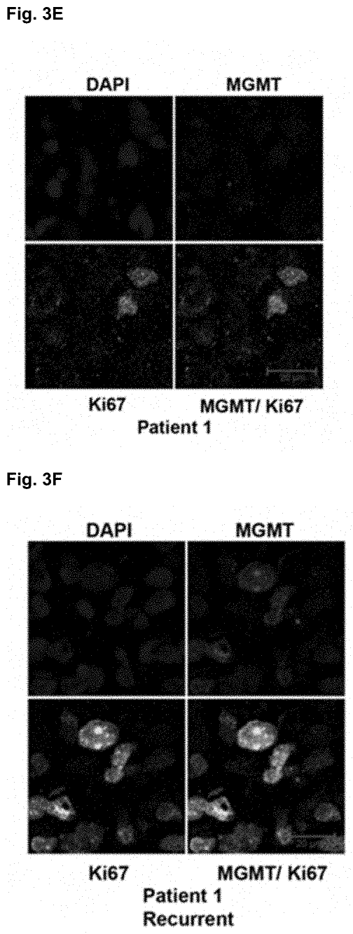

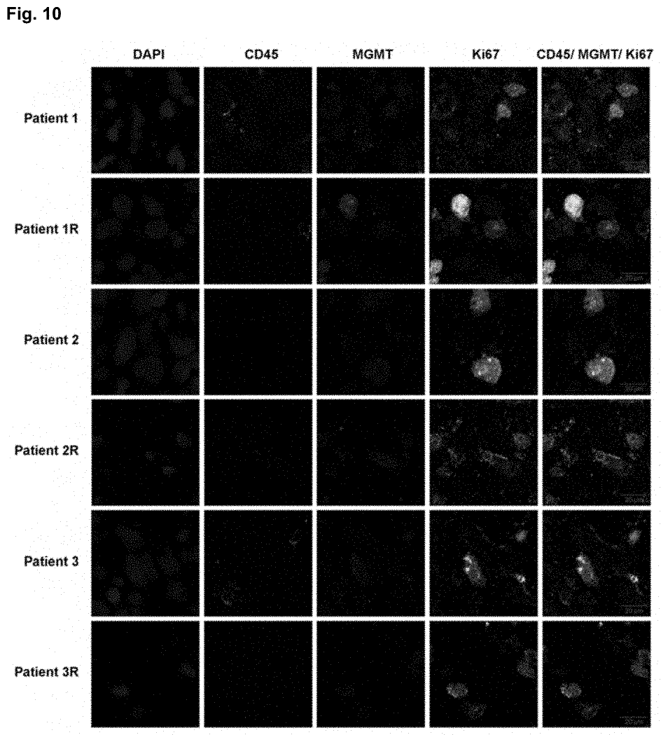

To determine whether enhancer activation can also be detected in primary tissues, we chose to analyze H3K4me1 and H3K27ac at the enhancer locus in paired primary and recurrent GBM tumors with a methylated MGMT promoter. Sixty four paired frozen patient tumor samples at Mayo Clinic were identified and further filtered based on presence of MGMT promoter hypermethylation, TMZ treatment prior to resection of recurrent disease and the suitable tumor cell density and tissue quality to yield three pairs for subsequent ChIP-qPCR analysis (Table 4). Compared to the primary tumor, both H3K4me1 and H3K27ac levels increased in the recurrent tumor from patient #1 (FIGS. 3A-3B). In contrast, while H3K27ac increased slightly in the recurrent tumor from patient #2 and #3, the enrichment of H3K4me1 was not altered at their enhancer locus. Interestingly, we detected a significant increase in MGMT expression in the recurrent tumor from patient 1 compared to its matched primary tumor (FIG. 3C), whereas MGMT expression was not significantly altered in recurrent tumors from patients #2 and #3. Similarly, the fraction of MGMT expressing and proliferating tumor cells increased only in recurrent tumor from patient #1, while this fraction of cells was not observed in tumors from patient #2 and #3 (FIGS. 3D-3F, FIG. 10). Although the small sample size precludes robust conclusions, the data suggests activation of the novel enhancer may also drive the MGMT expression in some recurrent tumors with MGMT promoter methylation.

TABLE 4
Treatment information for patients who
provided primary and recurrent tumor samples.

| Demographics | Patient 1 | Patient 2 | Patient 3 |
|---|---|---|---|
| Age | 64 | 67 | 68 |
| Sex | Male | Male | Female |
| Grade | IV | IV | IV |
| MGMT status | methylated | methylated | methylated |
| Treatment | Subtotal | Gross total | Aggressive |
| | resection | resection | resection |
| | 60 Gy + TMZ | 60 Gy + TMZ | 60 Gy + TMZ |
| | adjuvant | adjuvant | adjuvant |
| | TMZ × 6 cyc | TMZ × 6 cyc | TMZ × 6 cyc |
| | | Enrolled in | |
| | | N0877 trial | |
| 1^{st} disease free | 4.7 mo | 11.3 mo | 15.6 mo |
| interval* | | | |
| Salvage therapy | TMZ × 6 cyc | TMZ × 12 cyc | TMZ × 6 cyc |
| 2^{nd} disease free | 0 mo | 4 mo | 0 mo |
| interval** | | | |
| Treatment after | 50 Gy + | CCNU × 6 | CCNU × 1 |
| second | TMZ | mo Then | cycle Then |
| resection | Then 3 | 35 Gy + | CCNU & |
| | mo later | Avastin | Avastin × |
| | Avastin | | 8 mo |
| | | | Then 35 Gy |
| Overall survival | 29 months | 55 months | 43 months |

*Interval from the end of prior treatment to radiographic progression.
**Interval from the end of prior treatment to resection.

The patient demographics and treatment records were summarized in this table.

### Example 5

### Increased Acetylation at the Enhancer Increases MGMT Expression In Vitro

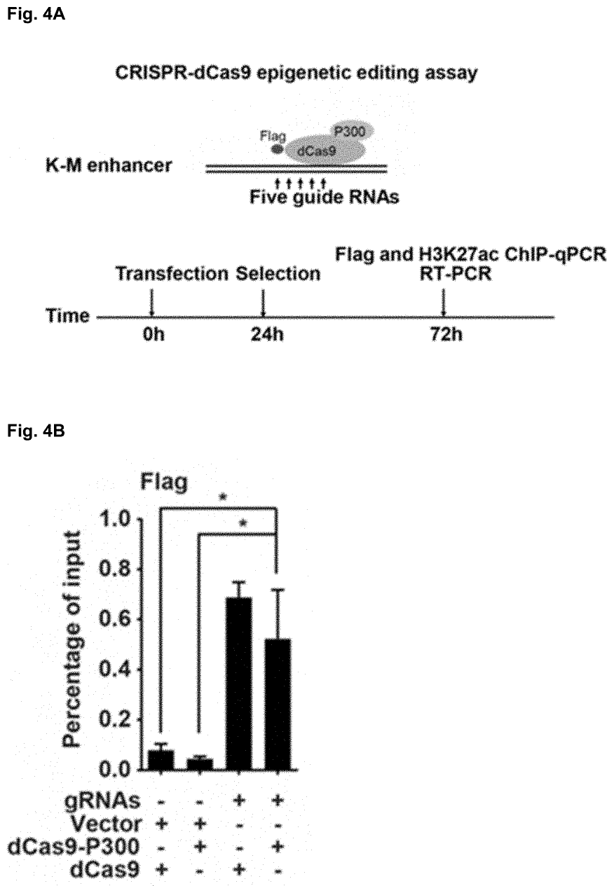

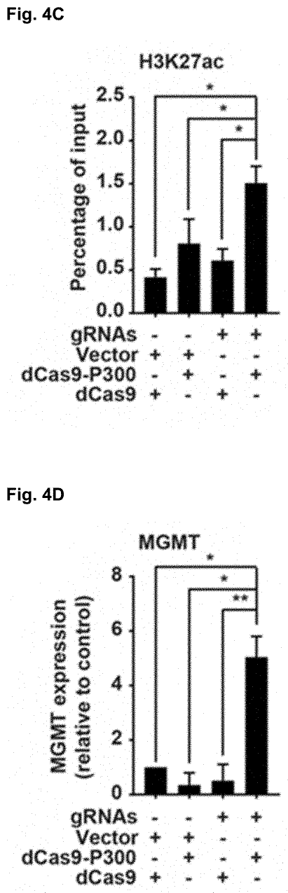

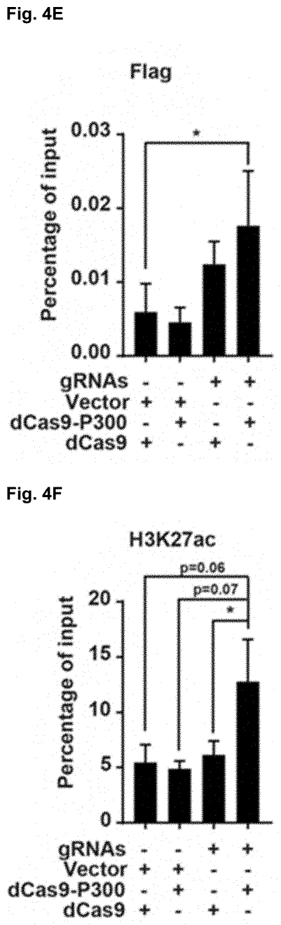

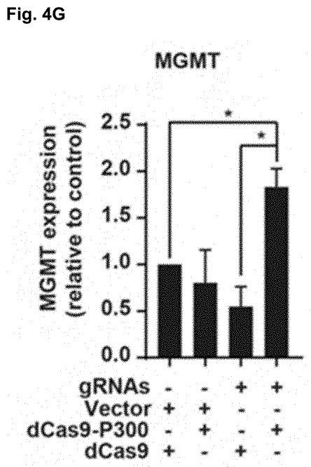

The level of H3K27ac at the enhancers is known to correlate with enhancer activity and gene expression. Therefore, we tested whether an increase in H3K27ac on nucleosomes surrounding the enhancer will affect MGMT expression by targeting the catalytic domain of histone acetyltransferase p300 to the enhancer locus (Hilton et al. 2015). Briefly, a Flag-tagged nuclease deactivated Cas9 (dCas9) protein was fused with p300 HAT domain and was targeted to the enhancer locus using five guide RNAs (gRNAs) in cells with low levels of MGMT expression (FIG. 4A). HEK293T cells were chosen first in this experiment due to their high transfection efficiency. Successful targeting of dCas9 or dCas9^{p300 Core} fusion proteins to the K-M enhancer locus was confirmed by Flag ChIP-qPCR (FIG. 4B). Moreover, we observed a marked increase of H3K27ac at the K-M enhancer locus when dCas9^{p300 Core} fusion protein, but not dCas9 alone, was expressed along with gRNAs (FIG. 4C). RT-PCR analysis indicated MGMT transcription also increased dramatically in HEK293T cells when targeting dCas9^{p300 Core} to the enhancer region by CRISPR/dCas9 (FIG. 4D). Although expression of the dCas9^{p300 Core} fusion protein alone without gRNAs slightly increased H3K27ac levels, this increase did not significantly alter MGMT expression, suggesting that a threshold level of H3K27ac is needed to alter the chromatin state of the enhancer and gene expression of MGMT. We also observed a slight, but significant increase in MGMT expression after we targeted dCas9^{p300 Core} to the enhancer locus in the 5199 line (FIGS. 4E-4G). Thus, the increased H3K27ac at nucleosomes surrounding the K-M enhancer can stimulate MGMT expression even in the presence of MGMT promoter methylation.

### Example 6

### Enhancer Depletion Reduces MGMT Expression and Increases TMZ Sensitivity

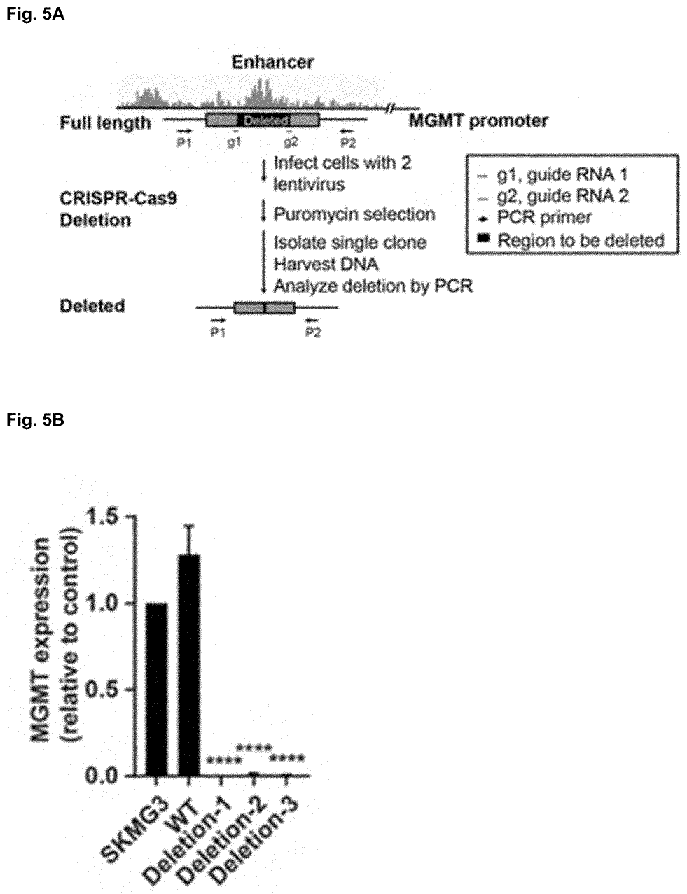

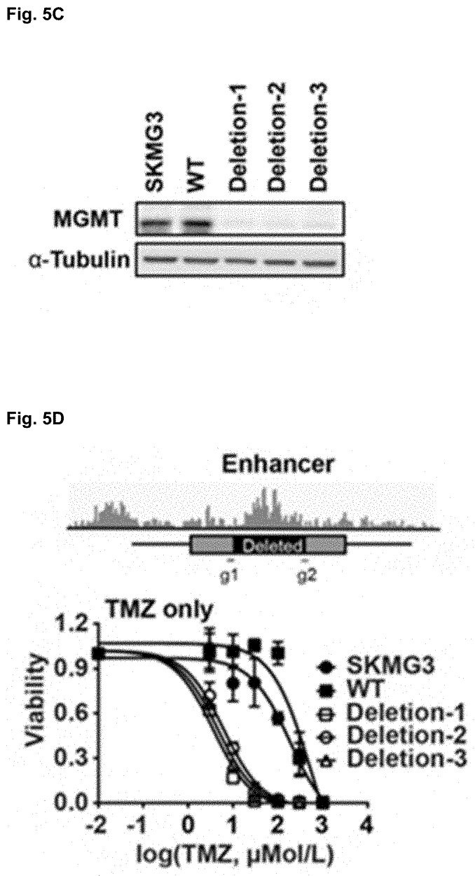

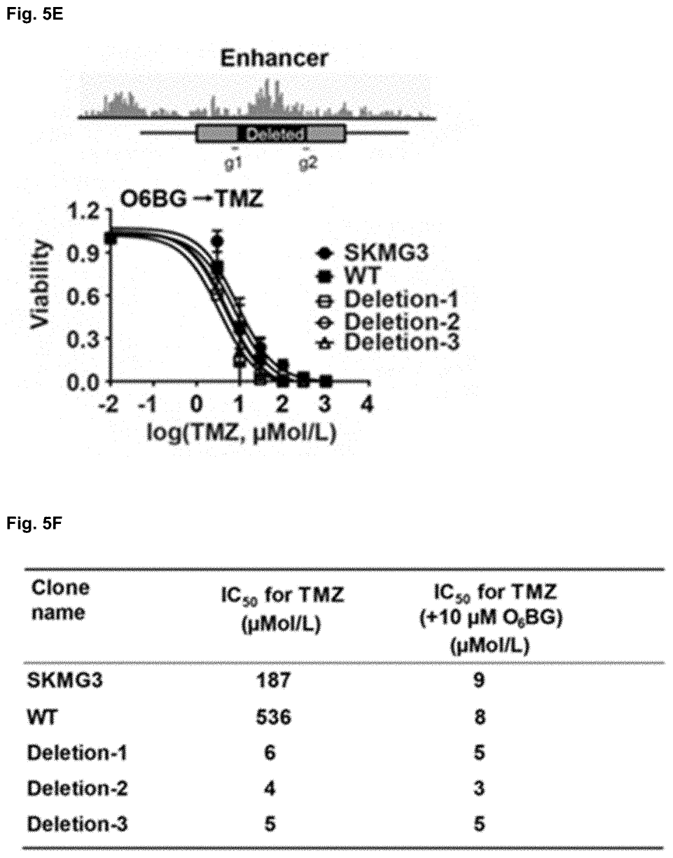

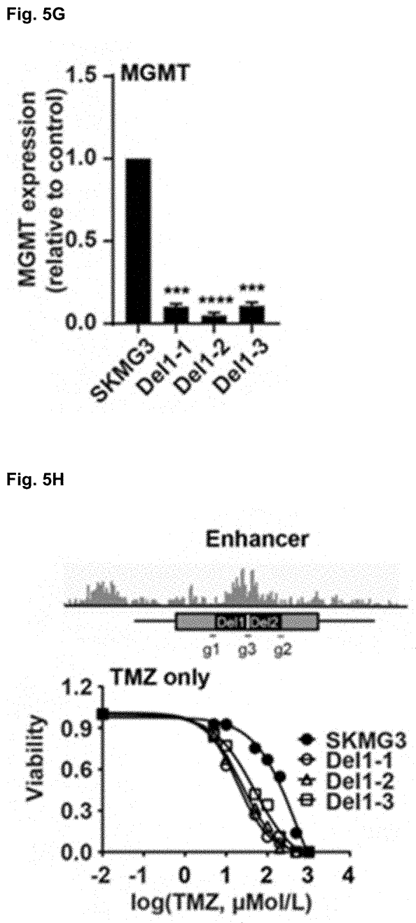

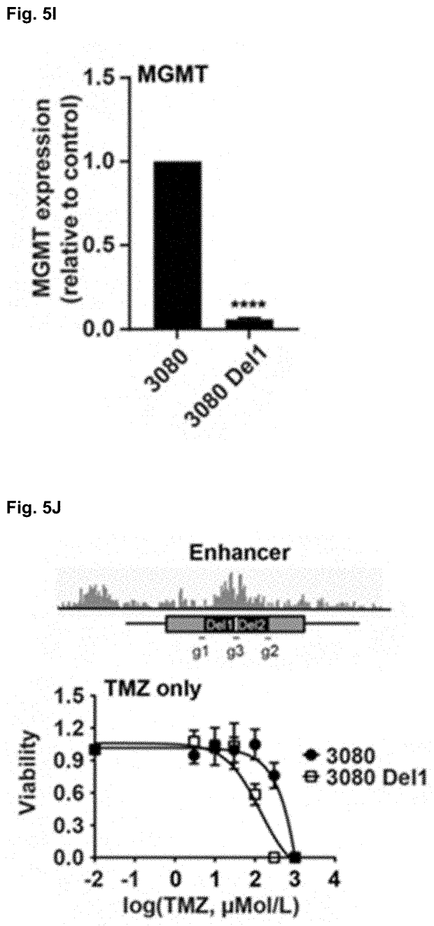

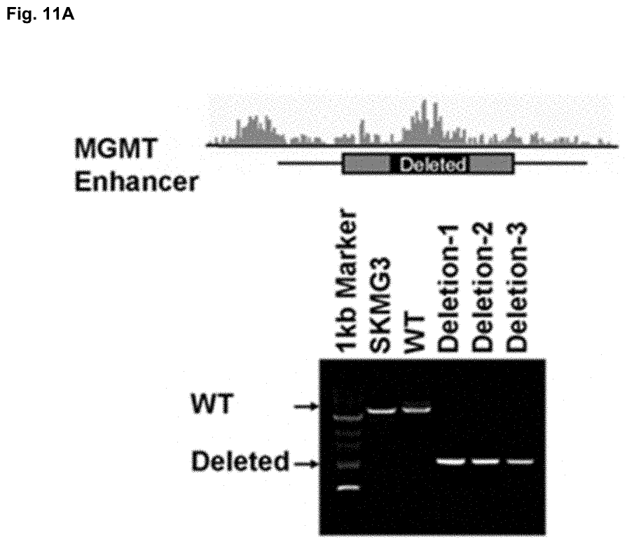

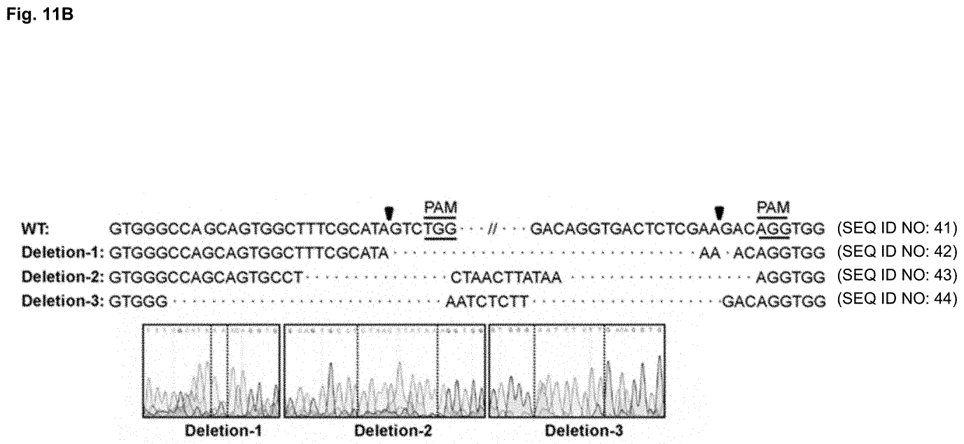

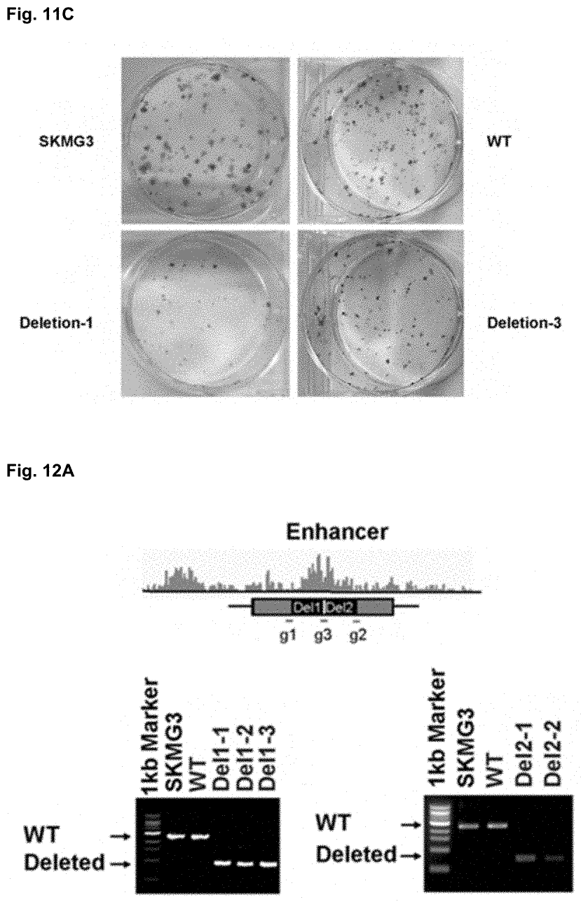

To directly determine whether the enhancer was essential for MGMT expression, we used the CRISPR/Cas9 system to delete a 3.3 kb region (chr10: 130,704,894-130,708,206) in the MGMT-expressing SKMG3 glioblastoma cell line (FIG. 5A) (Konermann et al. 2015; Ran et al. 2013). Four independent clones (three homozygous deletion clones and one wild-type control clone) were isolated. The genotypes of those four clones were identified by PCR and further confirmed by Sanger sequencing (FIGS. 11A-11B). Compared to parental SKMG3 cells and wild type control clone, MGMT transcription and protein expression level were markedly reduced in enhancer-deleted clones (FIGS. 5B-5C).

Next, we analyzed the TMZ sensitivity of SKMG3 clones using the clonogenic assay. Compared to parental SKMG3 lines and wild type control clone, the enhancer deleted clones were significantly more sensitive to TMZ treatment, with a dramatic log-fold decrease in IC₅₀ from over 100 μM to less than 10 μM (FIGS. 5D-5F). The sensitizing effect was not observed in the group pre-treated with the MGMT inhibitor O₆BG (FIGS. 5E-5F), indicating the change in TMZ sensitivity was mechanistically linked to MGMT activity. Since this TMZ concentration range is clinically achievable, these data suggest that efficient suppression of the enhancer activity could be a novel strategy to overcome the emergence of TMZ resistance.

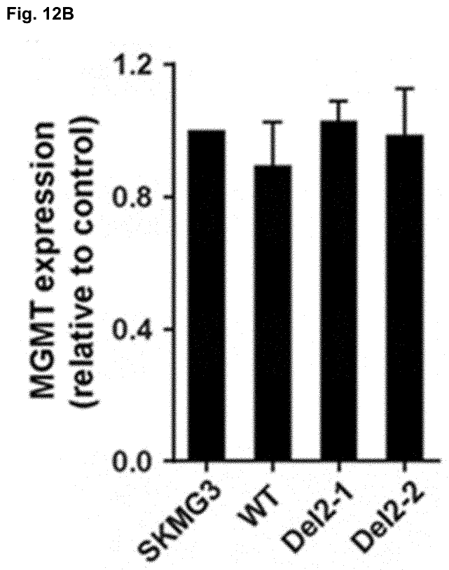

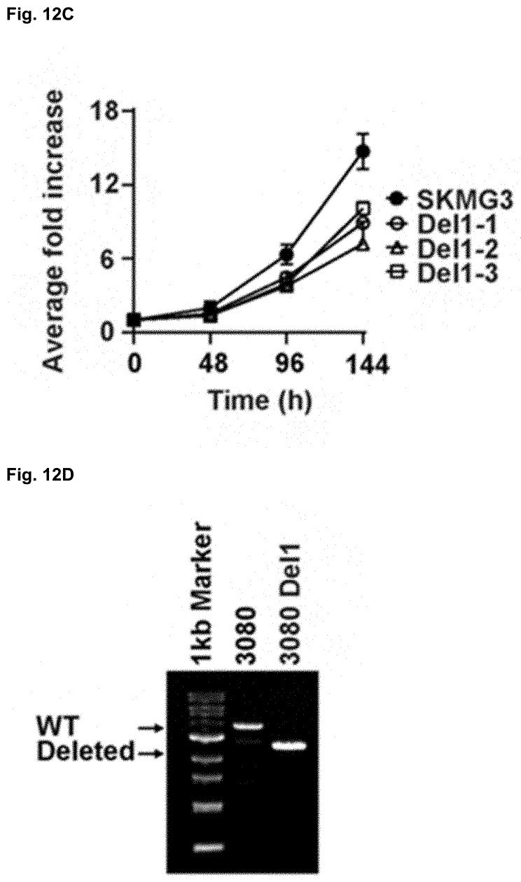

To further narrow down the location of the enhancer activating the MGMT expression in SKMG3 cells, we designed an additional guide RNA (g3) targeted at the middle of the other two guide RNAs used for the 3.3 kb deletion and generated two different 1.5 kb deletions when combined with g1 or g2 guide RNAs, respectively (FIG. 12A). Depletion of the first 1.5 kb region (ch10: 130,704,894-130,706,549), named Del1 region, also resulted in a dramatic reduction in MGMT suppression in SKMG3 cells (FIG. 5G), while deletion of the Del2 region, the 1.8 kb region more closer to the MGMT promoter, did not cause a significant change in MGMT expression (FIG. 12B). This result further narrowed down the location of K-M enhancer to a 1.5 kb region that lies about 560 kb away from the MGMT promoter that is essential for MGMT expression. Similar to larger deletion clones, SKMG3 cells with the 1.5 kb deletion also showed increased TMZ sensitivity, but to lesser degree than deletion clones with the 3.3 kb region (FIG. 5H), suggesting that the second half of the 3.3 kb region also contributes to the regulation of MGMT expression. Finally, we deleted the 1.5 kb Del1 fragment from the 3080 line and obtained one clone (FIG. 12D). Deletion of this fragment in the 3080 cells also resulted in reduced MGMT expression (FIG. 5I) and increased TMZ sensitivity in vitro (FIG. 5J).

### Example 7

### Deletion of the Enhancer Leads to Impaired Proliferation and Reduced Expression of Ki67

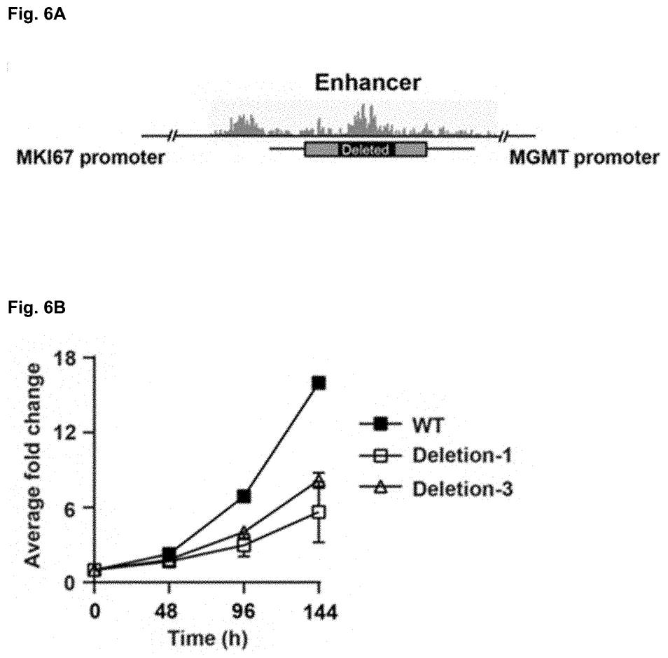

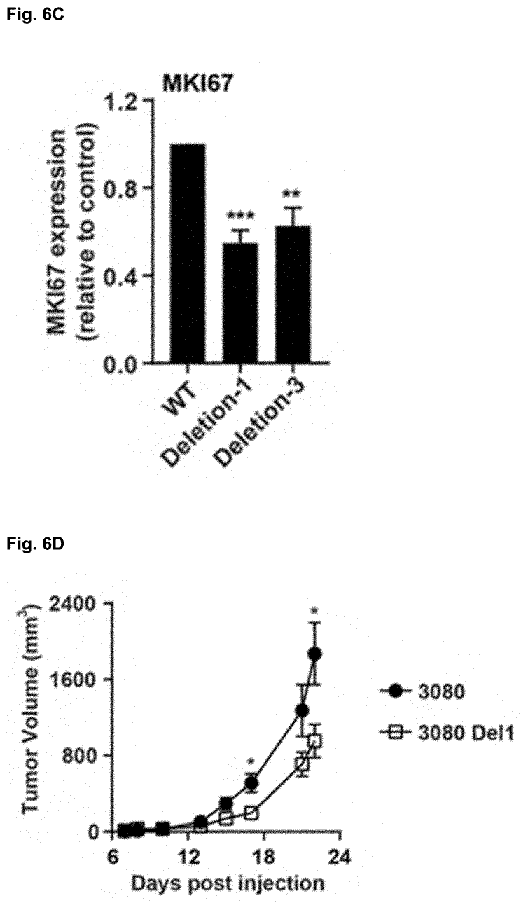

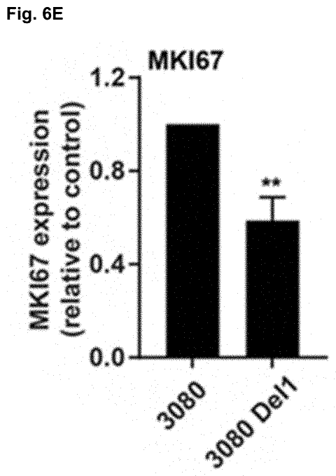

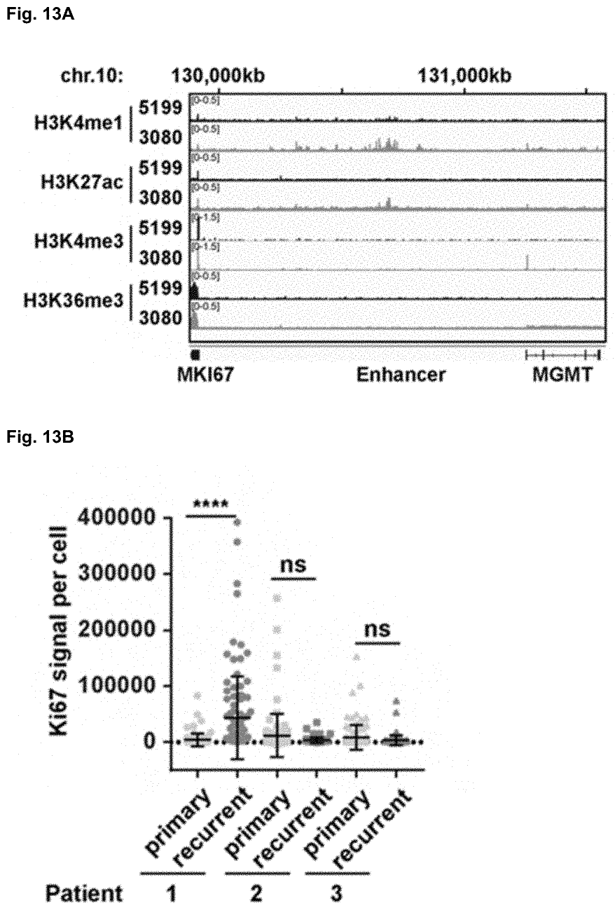

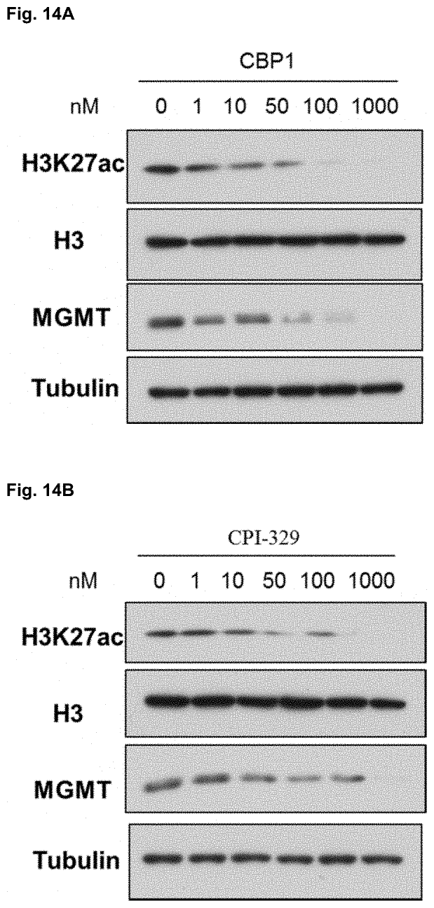

K-M enhancer may not only regulate protein expression but also promotes proliferation. During clonogenic assay analysis, the untreated deletion clones formed smaller colonies suggestive of impaired proliferation (FIG. 11C). This observation was confirmed by a proliferation assay, showing a clear decrease in proliferation rate in two out of three 3.3 kb deletion and all 1.5 kb deletion SKMG3 clones (FIGS. 6A-6B, FIG. 12C). This suggests that the enhancer may also regulate expression of genes involved in proliferation in addition to MGMT. Because the enhancer resides between MGMT and MKI67, a gene encoding the nuclear protein Ki67 that serves as a proliferation marker for many tumors including GBM (FIG. 13A), we analyzed the expression of Ki67 in enhancer deleted clones. In SKMG3 enhancer deleted clones, the two 3.3 kb deletion clones which have reduced proliferation rates also showed a significant decrease in Ki67 expression (FIG. 6C). These same results were recapitulated in the 3080 Del1 enhancer deleted clone (FIGS. 6D-6E). Consistent with those findings, an increase in Ki67 was observed in the recurrent tumor from patient #1 with the activated enhancer (FIG. 13B). Collectively, our results indicate that the K-M enhancer, likely regulates both the expression of Ki67 and MGMT to control both cell proliferation and TMZ sensitivity.

### Example 8

### A New Plausible Strategy for the Design of Novel Biomarkers and Treatments Strategies for Cancers

Previous studies have associated MGMT hypermethylation with gene silencing (Watts et al. 1997). However, significant discordance between promoter hypermethylation and MGMT suppression are observed both in vitro and in vivo (Kitange et al. 2012; Wang et al. 1992). Here we identify a novel distal enhancer (K-M enhancer) regulating MGMT expression. Surprisingly, promoter hypermethylation does not suppress enhancer-mediated MGMT expression, suggesting the mechanism revealed in this study might contribute to the reported discordance between methylation and expression in at least a fraction of tumors. Deletion of the K-M enhancer reduces MGMT and Ki67 expression, decrease cell proliferation, and sensitizes cells to TMZ to a clinical relevant level, which suggests potential therapeutic benefits of targeting enhancer activity. Beyond MGMT, a set of 1141 novel putative enhancers were activated in the TMZ resistant 3080 line (Table 5, in which the genomic location of each putative enhancer in Group-1 cluster, shown in FIGS. 1A-1I, was listed. The H3K4me1 and H3K27ac change on each putative enhancer locus was also listed together with the calculation of significance. The mRNA ID for the RNA transcript of the gene closest to each putative enhancer was listed in the second column from the right. The fold change of expression for each transcript was listed in the first column for the right, calculated using the following formula: RNA-seq reads in 3080 line/RNA-seq reads in 5199 line.), which raises the possibility that multiple enhancers are altered in response to TMZ therapy and that more than one enhancer element may contribute to the emergence of drug resistance.

MGMT expression is mechanistically linked to TMZ resistance, and the discordance between promoter methylation and protein expression observed in a subset of patients limits the prognostic accuracy of methylation assessment. Despite the overt requirement for protein expression of MGMT to repair TMZ-induced damage, MGMT promoter methylation is a more accurate predictor of TMZ resistance as compared to either RNA or protein expression. However, the predicted favorable outcome was not observed in 25% of MGMT hypermethylated patients, who exhibited de novo resistance and was progressed within 9 month under TMZ therapy (Hegi et al. 2005). In this study, we demonstrate that activation of the K-M enhancer can drive high level MGMT expression despite promoter methylation. Moreover, a subset of MGMT hypermethylated GBM PDX models with K-M enhancer activation expresses basal MGMT protein in association with de novo TMZ resistance. These observations may help explain why approximately a quarter of newly diagnosed, MGMT hypermethylated GBM patients, progress within the first few months of TMZ therapy. Finally, the activation of the K-M enhancer during resistance emergence without corresponding changes to MGMT promoter methylation may partially explain the poor prognostic performance of methylation status in recurrent GBM. Although mechanisms of inherent and acquired TMZ resistance extend beyond MGMT regulation, we might anticipate that assessment of epigenetic states for both the promoter and enhancer may provide a more robust and accurate predictive biomarker for TMZ sensitivity.

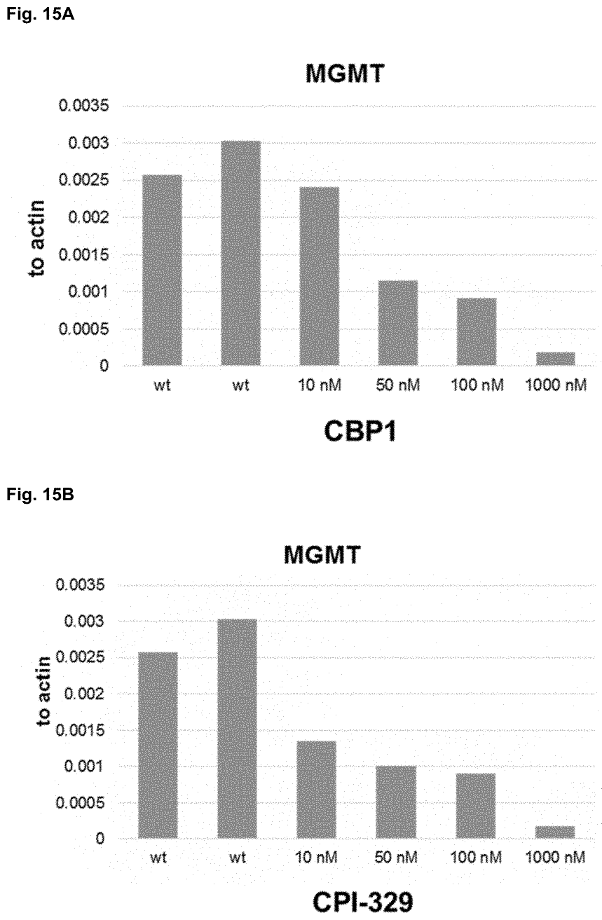

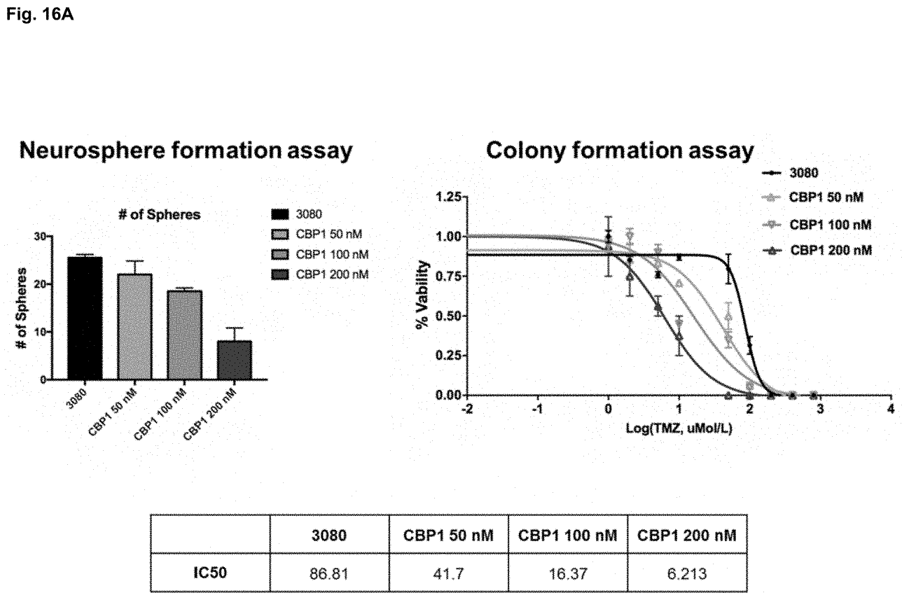

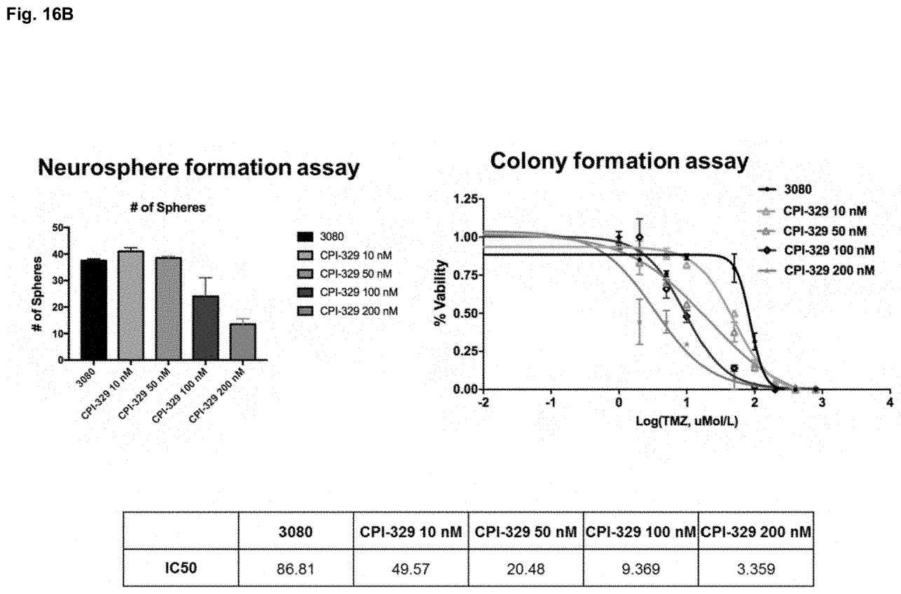

Beyond enhanced prognostic accuracy, therapeutic suppression of K-M enhancer could be used to delay the emergence of TMZ resistance and/or sensitize resistant patients to TMZ. We showed that epigenetic activation of the K-M enhancer drives TMZ resistance, while enhancer deletion results in greater TMZ sensitivity. Similarly, blocking enhancer activation may prevent enhancer driven TMZ resistance. A similar concept has been tested in breast and lung cancer, where epigenetic inhibitors successfully prevented chemo-resistance emergence driven by epigenetic alterations of gene promoters (Gardner et al. 2017; Meisenberg et al. 2017). If successful, enhancer inhibition may prevent or delay the emergence of TMZ resistance and produce more durable responses for GBM patients, which may make a critical improvement in survival. Furthermore, deletion of the enhancer reduces proliferation and sensitizes cells to TMZ. This indicates that blocking K-M enhancer activity potentially not only enhances TMZ response, but also reduces aggressiveness of otherwise TMZ resistant tumors. In principle, enhancer activity can be blocked by several methods including blocking the recognition of acetylated histones and inhibition of histone acetyltransferase activity. For example, both Bromodomain inhibitors (BETi), which blocks H3K27ac recognition by Bromodomain containing proteins, and histone acetyltransferases inhibitors (HATi), which reduce enhancer activity by inhibiting H3K27 acetylation, can effectively inhibit enhancer-driven transcription activation with acceptable toxicity (Muller et al. 2011; Wadhwa et al. 2016; Milite et al. 2015). We have shown that a combination of p300/CBPi with TMZ resulted in reduced MGMT expression (FIGS. 15A and 15B) and increased TMZ sensitivity in vitro (FIGS. 16A and 16B). These results suggest that a combination of p300/CBPi or BETi with TMZ may delay the emergence of TMZ resistance in responsive tumors and potentially sensitize the drug resistant patients to TMZ.

In contrast to BET and HAT inhibitors, histone deacetylase inhibitors (HDACi), which globally increase histone acetylation including H3K27ac, may promote the emergence of TMZ resistance by activating the K-M enhancer. Supporting this ides, we observed that combination treatment of SAHA, an FDA-approved HDACi, with TMZ specifically promotes elevation of MGMT expression as a mechanism of TMZ resistance (Kitange et al. 2012). Our enhancer activation model suggests that acetylation of both the MGMT promoter and the K-M enhancer could be a major cause of this effect. Thus, any future designs of treatment strategies that combine TMZ and histone deacetylase inhibitors should be approached with appropriate caution.

Beyond mediating MGMT expression and TMZ sensitivity, enhancer deletion was associated with significantly reduced proliferation in five out of six enhancer deleted clones, suggesting that K-M enhancer inactivation may also reduce tumor growth. Interestingly, unlike the observed increase in MGMT expression, Ki67 expression in 5199 was similar to 3080 line, indicating differential influence of the K-M enhancer on the two most proximal genes, MGMT and MKI67. One possible explanation for this would be if the enhancer-promoter interactions for MGMT and MKI67 are mediated by different transcription factors. Alternatively, it is possible that some PDX lines including 3080 have sequence changes at this K-M enhancer region. This change either creates a binding site for transcription activators or destroys a binding site for transcription repressors. Future studies are needed to catalog the transcription factors that bind to the K-M enhancer. Taken together, the K-M enhancer inhibition may not only reduce MGMT expression but also reduce tumor aggressiveness.

In summary, our study reveals a previously undocumented enhancer that, when activated, promotes MGMT expression and TMZ resistance in different cell lines, even in the presence of promoter methylation. This enhancer also regulates the expression of Ki67 to influence tumor aggressiveness. Loss of this enhancer is associated with increased TMZ sensitivity and reduced proliferation. These findings suggest that inhibition of enhancer activity is a new plausible strategy for the design of novel biomarkers and treatments strategies for cancers.

TABLE 5
The original list of Group-1 putative enhancers.

| chromo- | | | | H3K4me1 | | H3K27ac | | | RNAseq |
|---|---|---|---|---|---|---|---|---|---|
| some | star | end | strand | (3080/5199) | p-value | (3080/5199) | p-value | adjacent gene | (3080/5199) |
| chr10 | 1.31E+08 | 1.31E+08 | + | 2.63 | 0.27294 | 3.35 | 7.28E−15 | NM_002412 | 15.63 |
| chr10 | 1.31E+08 | 1.31E+08 | + | −0.04 | 0.956481 | 4.52 | 5.98E−07 | NM_002412 | 15.63 |
| chr17 | 15157604 | 15157732 | + | 0.57 | 0.230388 | 2.38 | 4.45E−08 | NR_039884 | 7.57 |
| chr19 | 13974673 | 13974801 | + | 0.13 | 0.464479 | 2.41 | 3.22E−05 | NR_029613 | 7.49 |
| chr1 | 84012538 | 84012744 | + | 0.23 | 0.02581 | 1.98 | 7.12E−06 | NR_049805 | 6.64 |
| chr11 | 75051486 | 75052116 | + | 1.04 | 0.06437 | 1.37 | 2.17E−08 | NR_029891 | 6.13 |
| chr13 | 80380579 | 80380870 | + | −0.68 | 0.029802 | 1.49 | 2.76E−05 | NR_046685 | 5.91 |
| chr8 | 53341449 | 53341663 | + | 2.26 | 0.065139 | 2.64 | 2.45E−06 | NM_014682 | 5.64 |
| chrY | 6331521 | 6331671 | + | 0.96 | 0.003193 | 4.4 | 2.04E−06 | NR_003591 | 4.91 |
| chr10 | 49916455 | 49916676 | + | 2.28 | 0.010657 | 2.34 | 1.37E−05 | NM_020945 | 4.32 |
| chr17 | 34092496 | 34092624 | + | 0.92 | 0.152933 | 2.15 | 9.64E−05 | NM_145272 | 4.32 |
| chr4 | 54501374 | 54501502 | + | 1.26 | 0.156387 | 2.88 | 3.48E−05 | NR_046827 | 4.32 |
| chr7 | 69852399 | 69852616 | + | 0.81 | 0.93262 | 2.11 | 3.97E−05 | NM_022479 | 4.32 |
| chr1 | 18014635 | 18014807 | + | 2.04 | 0.099329 | 3.05 | 2.00E−07 | MM_030812 | 3.32 |
| chr1 | 2.34E+08 | 2.34E+08 | + | 1.38 | 0.002207 | 1.7 | 8.33E−11 | NM_032435 | 3.22 |
| chr1 | 2.34E+08 | 2.34E+08 | + | 2.16 | 0.016043 | 2.17 | 1.66E−06 | NM_032435 | 3.32 |
| chr10 | 98878556 | 98876785 | + | −1.54 | 0.103108 | 3.38 | 1.29E−07 | NR_038330 | 3.32 |
| chr10 | 1.07E+08 | 1.07E−08 | + | 0.52 | 0.044377 | 1.36 | 4.82E−15 | NM_014978 | 3.32 |
| chr17 | 1157260 | 1157466 | + | −0.96 | 0.104912 | 2.34 | 1.37E−05 | NM_001164405 | 3.32 |
| chr18 | 29161447 | 29161805 | + | −0.03 | 0.460491 | 1.83 | 1.43E−07 | NM_000371 | 3.32 |
| chr18 | 40238107 | 40238433 | + | 0.63 | 0.261697 | 2.39 | 1.03E−11 | NM_002930 | 3.32 |
| chr18 | 53958769 | 53958980 | + | 0.23 | 0.391248 | 1.62 | 3.67E−05 | NR_040026 | 3.22 |
| chr2 | 1.5E+08 | 1.55+08 | + | 0.46 | 0.463277 | 1.77 | 4.13E−05 | NM_177964 | 3.32 |
| chr20 | 45192532 | 45192824 | + | −0.93 | 3.038183 | 1.48 | 4.35E−07 | NM_080721 | 3.32 |
| chr20 | 46756813 | 46757250 | + | −0.3 | 0.29184 | 1.33 | 2.91E−06 | NR_228958 | 3.32 |
| chr20 | 46831807 | 46831971 | + | 0.46 | 0.463277 | 3.46 | 4.08E−08 | NR_026658 | 3.32 |
| chr3 | 38786368 | 38786977 | + | 3.26 | 2.23E−05 | 2 | 2.30E−11 | NM_006514 | 3.32 |
| chr4 | 94337656 | 94338038 | + | −1.13 | 0.108085 | 1.71 | 2.84E−06 | NM_005172 | 3.32 |
| chr5 | 56752078 | 56752404 | + | −0.28 | 0.353275 | 2.2 | 2.71E−07 | NM_001017992 | 3.32 |
| chr5 | 56790567 | 56791307 | + | 1.04 | 0.013395 | 1.38 | 2.29E−08 | NM_001017992 | 3.32 |
| chr5 | 56797905 | 50798110 | + | 1.26 | 0.156387 | 2.01 | 4.17E−05 | NM_001017992 | 3.32 |
| chr9 | 1.04E+08 | 1.04E+08 | + | 1.78 | 0.052385 | 1.54 | 1.27E−05 | NM_147180 | 3.32 |
| chr8 | 1.44E+08 | 1.44E+98 | + | −1.13 | 0.065395 | 2.72 | 8.55E−07 | NM_001702 | 3.17 |
| chr19 | 8131466 | 8131777 | + | −0.65 | 0.045532 | 1.33 | 8.07E−06 | NM_005624 | 3 |
| chr17 | 45209 | 45345 | + | −0.04 | 0.956481 | 2.81 | 5.99E−05 | NM_003585 | 2.74 |
| chr17 | 81059143 | 81059279 | + | 1.46 | 0.222672 | 4.2 | 1.27E−05 | NM_001004431 | 2.32 |
| chr7 | 43139789 | 43140395 | + | 0.98 | 0.049001 | 2.08 | 2.70E−16 | NM_015052 | 2.32 |
| chr7 | 43147941 | 43148069 | + | −0.77 | 0.876277 | 2.13 | 6.02E−07 | NM_015052 | 2.32 |
| chr7 | 43151077 | 43151744 | + | 1.3 | 0.0345 | 2.13 | 1.38E−12 | NM_015052 | 2.32 |
| chr10 | 15335805 | 15337012 | + | 2.7 | 1.43E−10 | 2.82 | 3.92E−27 | NM_001010924 | 2.2 |
| chr10 | 15387046 | 15387750 | + | 2.39 | 7.41E−08 | 1.61 | 3.41E−09 | NM_001010924 | 2.2 |
| chr10 | 15394883 | 15395011 | + | −0.54 | 0.361994 | 2.26 | 1.64E−07 | NM_001010924 | 2.2 |
| chr10 | 15409910 | 15410105 | + | −1.13 | 0.188905 | 2.73 | 4.67E−10 | NM_001010924 | 2.2 |
| chr5 | 1.7E+08 | 1.7E+08 | + | −0.77 | 0.876277 | 4.34 | 3.77E−06 | NM_001034837 | 2.2 |
| chr5 | 1.7E+08 | 1.7E+08 | + | 0.73 | 0.02296 | 2.44 | 3.81E−13 | NM_001034837 | 2.2 |
| chr5 | 1.7E+08 | 1.7E+08 | + | 0.52 | 0.012348 | 1.31 | 2.22E−16 | NM_001034837 | 2.2 |
| chr5 | 1.7E+08 | 1.7E+08 | + | 1.46 | 0.010305 | 1.7 | 9.76E−05 | NM_001034837 | 2.2 |
| chr5 | 1.7E+08 | 1.7E+08 | + | 0.46 | 0.463277 | 2.31 | 8.78E−05 | NM_001034837 | 2.2 |
| chr5 | 1.7E+08 | 1.7E+08 | + | −0.22 | 0.311924 | 1.38 | 1.96E−10 | NM_001034837 | 2.2 |
| chr5 | 1.7E+08 | 1.7E+08 | + | 1.99 | 0.000726 | 2.24 | 1.36E−10 | NM_001034837 | 2.2 |
| chr5 | 1.7E+08 | 1.7E+08 | + | 1.59 | 0.000322 | 1.51 | 1.54E−10 | NM_001034837 | 2.2 |
| chr5 | 1.7E+08 | 1.7E+08 | + | 2.63 | 0.027294 | 1.6 | 9.18E−06 | NM_001034837 | 2.2 |
| chr3 | 1.35E+08 | 1.35E+08 | + | −0.54 | 0.329466 | 1.73 | 1.88E−05 | NM_004441 | 1.91 |
| chr4 | 1.41E+08 | 1.41E+06 | + | 2.46 | 0.042327 | 2.46 | 3.03E−06 | NM_018717 | 1.91 |
| chr15 | 69085288 | 69085426 | + | −0.13 | 0.374716 | 2.29 | 2.24E−05 | NR_036197 | 1.9 |
| chr15 | 69094025 | 69094269 | + | 0.04 | 0.492338 | 1.84 | 4.25E−05 | NR_036197 | 1.9 |
| chrX | 1.33E+08 | 1.33E+08 | + | 0.46 | 0.463277 | 2.65 | 2.30E−07 | NM_001164617 | 1.86 |
| chrX | 1.33E+08 | 1.33E+08 | + | −0.19 | 0.011157 | 3.2 | 7.38E−05 | NM_001164617 | 1.86 |
| chrX | 1.33E+08 | 1.33E+08 | + | 0.78 | 0.301098 | 1.63 | 2.81E−07 | NM_001164617 | 1.86 |
| chrX | 1.33E+08 | 1.33E+08 | + | 0.23 | 0.02581 | 2.11 | 397E−05 | NM_001164617 | 1.86 |
| chr12 | 15627091 | 15627820 | + | 2.26 | 0.000362 | 1.34 | 7.42E−07 | NM_030671 | 1.83 |
| chr4 | 1.58E+08 | 1.58E+08 | + | 4.73 | 5.59E−23 | 2.28 | 3.38E−16 | NM_001083620 | 1.78 |
| chr4 | 1.58E+08 | 1.58E+08 | + | 2.04 | 0.099329 | 3.68 | 7.23E−07 | NM_001083620 | 1.78 |
| chr4 | 1.58E+08 | 1.58E+08 | + | 0.78 | 0.077498 | 1.4 | 2.80E−07 | NM_001083620 | 1.78 |
| chr12 | 15508238 | 15508399 | + | 0.23 | 0.02581 | 3.29 | 4.03E−07 | NM_030667 | 1.7 |
| chr12 | 15504789 | 15510038 | + | 1.54 | 0.022876 | 2.71 | 7.11E−11 | NM_030667 | 1.7 |
| chr12 | 15570562 | 15571563 | + | 1.84 | 2.03E−05 | 1.65 | 2.72E−13 | NM_030667 | 1.7 |
| chr12 | 15574316 | 15574515 | + | 1.16 | 0.08465 | 3.92 | 3.74E−08 | NM_030667 | 1.7 |
| chr12 | 15575037 | 15575342 | + | 1.23 | 0.001123 | 1.88 | 4.69E−06 | NM_030667 | 1.7 |
| chr16 | 81674249 | 81674377 | + | −2.13 | 0.027712 | 4.2 | 1.27E−05 | NR_045112 | 1.58 |
| chr8 | 1.05E+08 | 1.05E+08 | + | 1.26 | 0.156387 | 1.71 | 7.60E−07 | NM_001385 | 1.58 |
| chr6 | 1.23E+08 | 1.23E+08 | + | 0.84 | 0.148331 | 2.36 | 6.32E−10 | NM_001010852 | 1.52 |
| chr6 | 1.23E+08 | 1.23E+08 | + | −0.77 | 0.876277 | 2.19 | 3.14E−07 | NM_001010852 | 1.52 |
| chr6 | 1.23E+08 | 1.23E+08 | + | 0.8 | 0.05977 | 1.79 | 3.37E−07 | NM_001010852 | 1.52 |
| chr16 | 55840982 | 55841158 | + | 1.54 | 0.022876 | 2.05 | 2.62E−05 | NM_001025194 | 1.5 |
| chr5 | 74173734 | 74173922 | + | −0.25 | 0.332095 | 2.06 | 6.83E−06 | NM_015586 | 1.47 |
| chrX | 1.33E+08 | 1.33E+08 | + | 1.78 | 0.003194 | 1.98 | 6.77E−15 | NM_001448 | 1.46 |
| chrX | 1.33E+08 | 1.33E+08 | + | 0.19 | 0.465306 | 1.82 | 4.67E−06 | NM_001448 | 1.46 |
| chrX | 1.33E+08 | 1.33E+08 | + | 2.04 | 0.099329 | 1.9 | 5.25E−07 | NM_001448 | 1.46 |
| chr8 | 4621157 | 4621293 | + | 1.34 | 0.000666 | 2.26 | 1.64E−07 | NM_033225 | 1.42 |
| chr21 | 40359192 | 40360744 | + | 0.68 | 0.026258 | 1.68 | 1.02E−17 | NM_005239 | 1.37 |
| chr21 | 40362390 | 40362524 | + | −0.22 | 0.38435 | 2.88 | 3.48E−05 | NM_005239 | 1.37 |
| chr1 | 17873576 | 17873755 | + | 0.35 | 0.325791 | 1.95 | 2.71E−05 | NM_018125 | 1.36 |
| chr20 | 36023210 | 36023597 | + | −0.37 | 0.217565 | 1.67 | 2.51E−09 | NM_198291 | 1.36 |
| chr4 | 40850203 | 40851064 | + | 2.32 | 1.41E−09 | 1.36 | 1.08E−09 | NM_001168051 | 1.36 |
| chr4 | 40851715 | 40851899 | + | 3.16 | 0.004461 | 2.24 | 9.42E−05 | NM_001166051 | 1.36 |
| chr4 | 40856855 | 40857235 | + | 1.7 | 0.01132 | 2.05 | 3.14E−07 | NM_001186051 | 1.36 |
| chr1 | 17892110 | 17892281 | + | 0.68 | 0.280156 | 1.68 | 6.15E−05 | NM_001011722 | 1.35 |
| chr1 | 17895787 | 17896416 | + | 1.25 | 0.000103 | 1.92 | 3.42E−13 | NM_001011722 | 1.35 |
| chr1 | 17900076 | 17900242 | + | 1.64 | 0.000139 | 3.01 | 3.58E−07 | NM_001011722 | 1.35 |
| chr1 | 17913886 | 17914567 | + | 1.87 | 5.91E−09 | 1.31 | 6.83E−09 | NM_001011722 | 1.35 |
| chr17 | 71477113 | 71477829 | + | 0.56 | 0.128974 | 2.43 | 2.81E−15 | NM_001144952 | 1.33 |
| chr17 | 71538702 | 71538899 | + | 1.38 | 0.025068 | 4.27 | 1.65E−10 | NM_001144952 | 1.33 |
| chr17 | 71546866 | 71547022 | + | 1.63 | 0.07633 | 3.29 | 4.03E−07 | NM_001144952 | 1.33 |
| chr17 | 71637475 | 71637752 | + | −0.54 | 0.3033 | 2.17 | 4.36E−07 | NM_001144952 | 1.33 |
| chr11 | 34294794 | 34295009 | + | 1.26 | 0.061907 | 2.2 | 3.96E−08 | NM_145804 | 1.28 |
| chr11 | 34321286 | 34321437 | + | 0.46 | 0.404925 | 2.29 | 2.24E−05 | NM_145804 | 1.26 |
| chr4 | 37931201 | 37931609 | + | −0.19 | 0.011157 | 1.49 | 2.76E−05 | NM_006607 | 1.26 |
| chr1 | 2.31E+08 | 2.31E+08 | + | 0.88 | 0.126482 | 1.87 | 3.59E−18 | NM_001258311 | 1.22 |
| chr1 | 2.31E+08 | 2.31E+08 | + | 0.04 | 0.504309 | 1.76 | 7.78E−10 | NM_001258311 | 1.22 |
| chr1 | 2.31E+08 | 2.31E+08 | + | 0.52 | 0.088482 | 1.42 | 8.20E−13 | NM_001258311 | 1.22 |
| chr20 | 50386254 | 50386386 | + | 0.87 | 0.210905 | 3.05 | 6.68E−06 | NM_006045 | 1.21 |
| chr3 | 25513336 | 25515881 | + | 0.81 | 0.000325 | 1.71 | 2.48E−31 | NM_000965 | 1.18 |
| chr16 | 84873786 | 84873915 | + | 0.46 | 0.463277 | 2.52 | 7.56E−05 | NM_031476 | 1.17 |
| chr3 | 63398978 | 63399527 | + | 0.22 | 0.369908 | 1.59 | 1.18E−08 | NM_144642 | 1.12 |
| chrX | 1.33E+08 | 1.33E+08 | + | 0.81 | 0.005284 | 2.86 | 6.62E−11 | NM_001101357 | 1.12 |
| chrX | 1.33E+08 | 1.33E+08 | + | 0.68 | 0.280156 | 2.25 | 3.66E−05 | NM_001101357 | 1.12 |
| chr6 | 71744515 | 71744826 | + | −0.28 | 0.353275 | 1.39 | 4.86E−05 | NM_080742 | 1.11 |
| chr6 | 71790599 | 71790937 | + | 0.33 | 0.277101 | 1.49 | 1.73E−05 | NM_080742 | 1.11 |
| chr8 | 1.01E+08 | 1.01E+08 | + | −0.18 | 0.381896 | 1.78 | 1.92E−06 | NM_003114 | 1.11 |
| chr12 | 94441501 | 94441629 | + | −0.54 | 0.361994 | 4.27 | 6.94E−06 | NM_005761 | 1.08 |
| chr22 | 26663920 | 26664258 | + | −0.42 | 0.23405 | 1.46 | 1.59E−05 | NM_001184774 | 1.06 |
| chr6 | 94118302 | 94118451 | + | −0.06 | 0.44608 | 3.2 | 1.25E−06 | NM_004440 | 1.03 |
| chr17 | 3864193 | 3664479 | + | −1.13 | 0.188905 | 1.85 | 1.26E−08 | NM_005173 | 1 |
| chr18 | 54540803 | 54541253 | + | −0.31 | 0.249034 | 1.67 | 9.22E−09 | NR_048536 | 1 |
| chr8 | 70378884 | 70279103 | + | −0.54 | 0.361994 | 3.08 | 3.76E−09 | NM_001128204 | 1 |
| chr8 | 70398889 | 70399134 | + | 0.81 | 0.93262 | 3.91 | 1.99E−11 | NM_001128205 | 1 |
| chr8 | 70399756 | 70399884 | + | 0.23 | 0.02581 | 2.49 | 1.21E−08 | NM_001128205 | 1 |
| chr2 | 1.05E+08 | 1.05E+08 | + | 0.46 | 0.308555 | 1.31 | 3.20E−05 | NR_015399 | 0.97 |
| chr2 | 1.08E+08 | 1.08E+08 | + | 0.78 | 0.391098 | 2.52 | 7.56E−05 | NM_001142352 | 0.94 |
| chr8 | 1.22E+08 | 1.22E+08 | + | 1.38 | 0.025068 | 2.03 | 1.32E−07 | NM_021021 | 0.93 |
| chr8 | 1.22E+08 | 1.22E+08 | + | 0.75 | 0.100892 | 1.79 | 7.19E−06 | NM_021021 | 0.93 |
| chr8 | 1.22E+08 | 1.22E+08 | + | −0.41 | 0.248943 | 2.12 | 1.03E−05 | NM_021021 | 0.93 |
| chr3 | 69869675 | 69870370 | + | 1.19 | 0.01248 | 2.28 | 3.38E−16 | NM_198177 | 0.91 |
| chr3 | 69870955 | 69871136 | + | 0.92 | 0.152933 | 4.05 | 6.20E−09 | NM_198177 | 0.91 |
| chr16 | 8635808 | 8636226 | + | 0.92 | 0.013438 | 1.31 | 3.20E−05 | NM_001146336 | 0.89 |
| chr21 | 33368894 | 33369446 | + | 1.63 | 0.00023 | 1.47 | 4.57E−09 | NR_038033 | 0.87 |
| chr21 | 33515567 | 33515782 | + | −2.28 | 0.004579 | 3.01 | 3.58E−07 | NR_038033 | 0.87 |
| chr1 | 1.12E+08 | 1.12E+08 | + | −0.83 | 0.033279 | 1.48 | 4.35E−07 | NM_172198 | 0.86 |
| chr9 | 1.03E+08 | 1.03E+08 | + | 1.78 | 0.149771 | 2.93 | 2.01E−05 | NM_001018116 | 0.86 |
| chr9 | 1.03E+08 | 1.03E+08 | + | −0.63 | 0.10124 | 2.06 | 8.33E−08 | NM_001018116 | 0.86 |
| chr2 | 84179635 | 84179923 | + | 1.04 | 0.010434 | 1.71 | 2.64E−06 | NR_003663 | 0.85 |
| chr5 | 13990775 | 13991016 | + | −0.08 | 0.429403 | 2.1 | 4.28E−06 | NM_001369 | 0.85 |
| chr9 | 1.13E+08 | 1.13E+08 | + | −1.09 | 0.016514 | 2.38 | 8.30E−06 | NM_001012993 | 0.85 |
| chr12 | 14075473 | 14075863 | + | 0.92 | 0.063595 | 1.35 | 2.50E−05 | NM_000834 | 0.84 |
| chr2 | 40447265 | 49447617 | + | 0.11 | 0.462988 | 2.38 | 5.51E−08 | NM_001112800 | 0.83 |
| chr2 | 40599364 | 40599574 | + | 0.87 | 0.210905 | 3.61 | 4.01E−09 | NM_001112800 | 0.83 |
| chr2 | 40662185 | 40662518 | + | 0.46 | 0.365076 | 2.82 | 9.15E−10 | NM_001112800 | 0.83 |
| chr5 | 64437302 | 64437564 | + | 0.46 | 0.463277 | 1.38 | 7.74E−05 | NM_197941 | 0.81 |
| chr2 | 5532734 | 5532957 | + | 3.21 | 3.53E−05 | 1.64 | 0.000358 | NM_003108 | 0.8 |
| chr4 | 81120300 | 81120428 | + | 0.45 | 0.015307 | 2.63 | 2.67E−05 | NM_001099403 | 0.8 |
| chr10 | 1.02E+08 | 1.02E+08 | + | −0.36 | 0.926622 | 1.68 | 6.15E−05 | NM_003988 | 0.79 |
| chr10 | 1.24E+08 | 1.24E+08 | + | 0.46 | 0.308555 | 1.84 | 4.25E−05 | NM_002775 | 0.79 |
| chr21 | 33263348 | 33263653 | + | 1.13 | 0.063706 | 1.73 | 1.68E−05 | NM_014586 | 0.78 |
| chr8 | 1.26E+08 | 1.26E+08 | + | 0.37 | 0.283356 | 2.2 | 1.53E−05 | NM_014751 | 0.78 |
| chr20 | 46391324 | 46392013 | + | 0.04 | 0.477655 | 1.39 | 8.78E−11 | NM_018837 | 0.75 |
| chr6 | 1.3E+08 | 1.3E+08 | + | 1.26 | 0.156387 | 3.52 | 4.18E−06 | NM_033515 | 0.75 |
| chr22 | 27459620 | 27460046 | + | 1.54 | 0.022876 | 2.1 | 3.04E−11 | NR_003491 | 0.74 |
| chr22 | 27469882 | 27470264 | + | 2.36 | 0.007037 | 1.6 | 4.58E−08 | NR_003491 | 0.74 |
| chr22 | 27504268 | 27505353 | + | 0.56 | 0.077732 | 1.61 | 2.35E−13 | NR_003491 | 0.74 |
| chr22 | 27507214 | 27507633 | + | 2.96 | 7.37E−06 | 1.51 | 4.68E−06 | NR_003491 | 0.74 |
| chr22 | 27511555 | 27512120 | + | 1.11 | 0.036985 | 1.9 | 1.63E−08 | NR_003491 | 0.74 |
| chr4 | 55540038 | 55540166 | + | −0.96 | 0.18457 | 3.2 | 7.36E−05 | NM_000222 | 0.74 |
| chr11 | 89802126 | 69803147 | + | 1.48 | 3.76E−05 | 1.53 | 4.00E−14 | NM_018043 | 0.73 |
| chr20 | 46423943 | 46424275 | + | 0.13 | 0.429293 | 1.4 | 1.21E−05 | NM_198596 | 0.73 |
| chr20 | 46505367 | 46505541 | + | 0.46 | 0.404925 | 1.7 | 2.56E−05 | NM_198596 | 0.73 |
| chr17 | 19170339 | 19170482 | + | 0.46 | 0.334119 | 2.78 | 5.42E−06 | NR_046824 | 0.68 |
| chr17 | 19182076 | 19182216 | + | 0.46 | 0.221407 | 1.96 | 6.59E−05 | NR_046824 | 0.68 |
| chr17 | 19182743 | 19183382 | + | 2.2 | 1.93E−10 | 1.42 | 1.23E−07 | NR_046824 | 0.68 |
| chr18 | 9813187 | 9813315 | + | −0.42 | 0.23405 | 2.01 | 4.17E−05 | NM_032243 | 0.68 |
| chr2 | 1.86E+08 | 1.86E+08 | + | 1.81 | 0.834416 | 2.13 | 6.02E−07 | NM_194250 | 0.67 |
| chr1 | 2.43E+08 | 2.43E+08 | + | 0.4 | 0.480721 | 1.38 | 1.96E−10 | NM_001195811 | 0.66 |
| chr1 | 2.43E+08 | 2.43E+08 | + | 3.82 | 6.26E−05 | 1.61 | 2.31E−05 | NM_001195811 | 0.68 |
| chr14 | 47861658 | 47861890 | + | −0.25 | 0.332095 | 1.48 | 1.09E−05 | NM_182830 | 0.66 |
| chr14 | 47936164 | 47936721 | + | 0.54 | 0.111688 | 1.48 | 6.99E−08 | NM_182830 | 0.66 |
| chr14 | 47970662 | 47977456 | + | 0.49 | 0.115226 | 1.42 | 3.11E−10 | NM_182830 | 0.68 |
| chr9 | 1.09E+08 | 1.09E+08 | + | −0.96 | 0.18457 | 3.05 | 6.68E−05 | NM_021224 | 0.65 |
| chr5 | 1.47E+08 | 1.47E+08 | + | 1.7 | 5.10E−08 | 1.33 | 1.19E−19 | NR_073101 | 0.63 |
| chr5 | 1.47E+06 | 1.47E+08 | + | 1.96 | 0.009351 | 1.88 | 3.41E−07 | NR_073101 | 0.63 |
| chr5 | 1.47E+08 | 1.47E+08 | + | 2.98 | 1.68E−07 | 1.85 | 6.47E−14 | NR_073101 | 0.63 |
| chr5 | 1.47E+08 | 1.47E+08 | + | 1.14 | 4.75E−06 | 1.55 | 3.36E−25 | NR_073101 | 0.63 |
| chr5 | 1.47E+08 | 1.47E+08 | + | 0.29 | 0.337406 | 1.38 | 6.92E−06 | NR_073101 | 0.63 |
| chr5 | 1.47E+08 | 1.47E+08 | + | 0.19 | 0.465306 | 2.34 | 2.39E−08 | NR_073101 | 0.63 |
| chr5 | 1.47E+08 | 1.47E+08 | + | 0.81 | 0.005384 | 2.86 | 6.02E−11 | NR_073101 | 0.63 |
| chr4 | 76955564 | 76956038 | + | 1.08 | 0.013198 | 1.6 | 4.58E−08 | NM_005409 | 0.59 |
| chr20 | 50582889 | 50583023 | + | 1.63 | 0.07633 | 1.91 | 1.75E−05 | NM_020436 | 0.58 |
| chr4 | 41543912 | 41544081 | + | 1.84 | 0.000139 | 2.15 | 9.64E−05 | NM_001112719 | 0.58 |
| chr5 | 7759550 | 7759941 | + | 1.65 | 0.028759 | 1.88 | 1.43E−07 | NM_001089584 | 0.58 |
| chr5 | 1.45E+08 | 1.45E+08 | + | 1.13 | 0.012899 | 2.3 | 1.03E−08 | NM_182960 | 0.58 |
| chr5 | 1.45E+08 | 1.45E+08 | + | 0.80 | 0.000372 | 1.72 | 7.66E−13 | NM_182960 | 0.58 |
| chr7 | 19955874 | 19956060 | + | 0.23 | 0.02581 | 2.05 | 2.62E−05 | NM_152774 | 0.58 |
| chr7 | 1.01E+08 | 1.01E+08 | + | −0.19 | 0.01157 | 3.1 | 3.83E−06 | NM_003378 | 0.58 |
| chr9 | 1.37E+08 | 1.37E+08 | + | 1.73 | 3.53E−05 | 1.68 | 1.62E−05 | NM_007101 | 0.58 |
| chr2 | 2.22E+08 | 2.22E+08 | + | 1.92 | 0.035602 | 2.07 | 6.37E−05 | NM_004438 | 0.57 |
| chr2 | 2.22E+08 | 2.22E+08 | + | 1.41 | 0.004736 | 1.59 | 5.79E−06 | NM_004438 | 0.57 |
| chr12 | 1.33E+08 | 1.33E+08 | + | −1.28 | 0.039667 | 2.39 | 2.01E−05 | NM_001142641 | 0.56 |
| chr10 | 15279576 | 15279729 | + | −0.54 | 0.361994 | 3.05 | 6.78E−06 | NM_004808 | 0.55 |
| chr11 | 7593182 | 7593676 | + | 2.23 | 4.07E−06 | 2.05 | 1.60E−12 | NM_001256568 | 0.55 |
| chr11 | 7600776 | 7601522 | + | 1.32 | 0.000664 | 2.1 | 3.04E−11 | NM_001256568 | 0.55 |
| chr11 | 7602134 | 7602568 | + | 3.26 | 2.23E−05 | 3 | 2.07E−11 | NM_001256568 | 0.55 |
| chr11 | 7603953 | 7604122 | + | 1.64 | 0.000139 | 2.41 | 3.22E−05 | NM_001256568 | 0.55 |
| chr11 | 7608273 | 7609591 | + | 2.77 | 4.01E−22 | 2.04 | 1.14E−31 | NM_001256568 | 0.55 |
| chr11 | 7651020 | 7051846 | + | 3.90 | 9.47E−13 | 1.9 | 2.45E−16 | NM_001256569 | 0.55 |
| chr11 | 7656210 | 7656676 | + | 1.73 | 0.001876 | 2.27 | 4.50E−14 | NM_001256569 | 0.55 |
| chr11 | 41399978 | 41400649 | + | 0.84 | 0.035899 | 1.31 | 5.42E−07 | NM_001258419 | 0.55 |
| chr4 | 1.21E+08 | 1.21E+08 | + | 0.94 | 0.113163 | 1.73 | 1.87E−06 | NM_033430 | 0.55 |
| chr4 | 1.21E+08 | 1.21E+08 | + | −0.22 | 0.38435 | 3.2 | 1.25E−06 | NM_001083 | 0.55 |
| chr16 | 77849568 | 77849832 | + | 1 | 0.022386 | 2.09 | 4.71E−07 | NM_020927 | 0.54 |
| chr16 | 77884557 | 77884685 | + | 1.04 | 0.218994 | 3.4 | 1.33E−05 | NM_020927 | 0.54 |
| chr22 | 27643727 | 27643911 | + | 0.46 | 0.463277 | 2.72 | 8.55E−07 | NM_002430 | 0.54 |
| chr22 | 27812662 | 27812979 | + | 0.04 | 0.529972 | 1.95 | 2.15E−07 | NM_002430 | 0.54 |
| chr22 | 28144856 | 28146329 | + | 0.16 | 0.427697 | 1.5 | 4.71E−07 | NM_002430 | 0.54 |
| chr8 | 97539032 | 97539160 | + | 1.04 | 0.156144 | 2.83 | 3.17E−06 | NM_002998 | 0.54 |
| chr18 | 43989632 | 43989832 | + | 3.36 | 0.00176 | 2.32 | 3.54E−07 | NM_152470 | 0.52 |
| chr7 | 15819961 | 15820159 | + | 0.66 | 0.121103 | 1.7 | 6.77E−06 | NM_005924 | 0.52 |
| chr8 | 1.21E+08 | 1.21E+08 | + | 1.22 | 0.007309 | 2.49 | 2.88E−07 | NM_014078 | 0.52 |
| chr8 | 1.22E+08 | 1.22E+08 | + | 0.52 | 0.08078 | 2.12 | 7.79E−08 | NM_014078 | 0.52 |
| chr18 | 13229002 | 13229379 | + | 0.16 | 0.381985 | 1.31 | 2.62E−06 | NM_181482 | 0.51 |
| chr2 | 74518776 | 74519077 | + | 0.92 | 0.152933 | 1.9 | 1.26E−06 | NM_021196 | 0.51 |
| chr5 | 15260987 | 15261346 | + | −0.54 | 0.245002 | 1.72 | 4.46E−06 | NM_012304 | 0.51 |
| chr8 | 70699186 | 70699321 | + | 1.19 | 0.113922 | 3.63 | 1.30E−06 | NM_001146009 | 0.51 |
| chr9 | 1.36E+08 | 1.36E+08 | + | −0.22 | 0.38435 | 1.43 | 9.39E−06 | NM_001042368 | 0.51 |
| chr9 | 1.36E+08 | 1.36E+08 | + | 1.28 | 0.156387 | 2.05 | 2.62E−05 | NM_001042368 | 0.51 |
| chr13 | 25709818 | 25710693 | + | 0.38 | 0.265527 | 1.93 | 1.05E−11 | NM_199138 | 0.5 |
| chr7 | 1.05E+08 | 1.05E+08 | + | 0.19 | 0.396995 | 2.07 | 6.37E−05 | NM_152749 | 0.5 |
| chr7 | 1.06E+08 | 1.06E+08 | + | −0.06 | 0.44608 | 2.51 | 1.16E−05 | NM_152749 | 0.5 |
| chr8 | 22927847 | 22928059 | + | 1.63 | 0.07633 | 2.49 | 2.88E−07 | NM_003842 | 0.5 |
| chrX | 1.3E+08 | 1.3E+08 | + | 0.81 | 0.079776 | 1.55 | 5.14E−05 | NM_182314 | 0.5 |
| chrX | 1.3E+08 | 1.3E+08 | + | 1.7 | 0.01132 | 3.19 | 1.89E−13 | NM_182314 | 0.5 |
| chrX | 1.3E+08 | 1.3E+08 | + | 2.92 | 0.011142 | 2.63 | 2.67E−05 | NM_182314 | 0.5 |
| chrX | 1.3E+08 | 1.3E+08 | + | 3.54 | 0.000686 | 2.86 | 6.62E−11 | NM_182314 | 0.5 |
| chrX | 1.3E+08 | 1.3E+08 | + | 1.78 | 0.003194 | 2.11 | 3.97E−05 | NM_182314 | 0.5 |
| chr17 | 20825376 | 20825725 | + | 0.68 | 0.126482 | 1.44 | 5.78E−06 | NM_001004306 | 0.49 |
| chr3 | 1.11E+08 | 1.11E+08 | + | −0.36 | 0.926622 | 2.68 | 1.45E−06 | NR_045114 | 0.49 |
| chr3 | 1.38E+08 | 1.38E+08 | + | 1.28 | 1.97E−09 | 1.61 | 1.88E−20 | NM_001252090 | 0.49 |
| chr5 | 1.54E+08 | 1.54E+08 | + | 1.25 | 0.000215 | 1.71 | 4.57E−25 | NM_198321 | 0.49 |
| chr7 | 87142891 | 87143294 | + | 1.63 | 0.07633 | 1.4 | 1.21E−05 | NM_018849 | 0.49 |
| chr8 | 85165622 | 85165750 | + | 2.26 | 0.065139 | 3.2 | 7.38E−05 | NM_001100391 | 0.48 |
| chr9 | 19481585 | 19482028 | + | 1.13 | 0.063706 | 1.6 | 1.46E−05 | NM_001010887 | 0.48 |
| chr13 | 78762313 | 78762466 | + | 1.04 | 0.325105 | 2.41 | 3.22E−05 | NR_047001 | 0.47 |
| chr2 | 1613290 | 1613612 | + | 0.19 | 0.424653 | 1.39 | 2.76E−06 | NM_012293 | 0.47 |
| chr8 | 1.21E+08 | 1.21E+08 | + | −0.81 | 0.170714 | 2.52 | 2.77E−08 | NM_021110 | 0.47 |
| chr8 | 1.21E+08 | 1.21E+08 | + | −0.04 | 0.956481 | 2.03 | 1.19E−06 | NM_021110 | 0.47 |
| chr4 | 73253915 | 73254058 | + | −0.11 | 0.394245 | 1.88 | 6.65E−05 | NM_014243 | 0.46 |
| chr4 | 73290340 | 73290510 | + | −1.02 | 0.010478 | 1.7 | 9.76E−05 | NM_014243 | 0.46 |
| chr7 | 55135496 | 55135624 | + | 1.16 | 0.000651 | 1.51 | 1.48E−09 | NM_201284 | 0.46 |
| chr16 | 11766248 | 11766572 | + | 0 | 0.521069 | 1.58 | 3.45E−05 | NM_003498 | 0.45 |
| chr19 | 46907361 | 46907664 | + | 0.67 | 0.062848 | 1.76 | 1.23E−06 | NM_032040 | 0.45 |
| chr19 | 46929003 | 46929166 | + | 1.46 | 0.032155 | 2.8 | 2.95E−07 | NM_032040 | 0.45 |
| chr19 | 46937902 | 46938046 | + | 1.46 | 0.109973 | 2.52 | 7.56E−05 | NM_032040 | 0.45 |
| chr12 | 28947573 | 28947924 | + | −0.06 | 0.437036 | 1.68 | 1.62E−05 | NM_001271783 | 0.44 |
| chr12 | 29395381 | 29395540 | + | 0.04 | 0.504309 | 3.09 | 1.18E−07 | NM_018099 | 0.44 |
| chr3 | 1.27E+08 | 1.27E+08 | + | 0.78 | 0.201565 | 2.41 | 3.22E−05 | NM_032242 | 0.44 |
| chr3 | 1.27E+08 | 1.27E+08 | + | −0.54 | 0.329468 | 4.52 | 5.98E−07 | NM_032242 | 0.44 |
| chr4 | 77817592 | 77817834 | + | −0.54 | 0.19189 | 2.98 | 1.96E−08 | NM_001029870 | 0.44 |
| chr5 | 9482727 | 9484410 | + | 1.12 | 1.00E−05 | 1.91 | 4.64E−29 | NM_003966 | 0.44 |
| chr8 | 1.46E+08 | 1.46E+08 | + | 0.46 | 0.334119 | 2.15 | 9.64E−05 | NM_213605 | 0.44 |
| chr11 | 1.17E+08 | 1.17E+08 | + | −0.23 | 0.201545 | 1.31 | 2.62E−08 | NM_020693 | 0.43 |
| chr10 | 60410762 | 60411324 | + | 0.9 | 0.083608 | 2.12 | 7.11E−11 | NR_027506 | 0.42 |
| chr10 | 60580636 | 60580764 | + | −0.77 | 0.876277 | 3.2 | 7.38E−05 | NR_027508 | 0.42 |
| chr18 | 22846936 | 22847453 | + | 0.94 | 0.005082 | 1.68 | 3.93E−09 | NM_015461 | 0.42 |
| chr18 | 22878853 | 22879021 | + | 0.46 | 0.286689 | 1.39 | 4.86E−05 | NM_015461 | 0.42 |
| chr19 | 35547644 | 35547696 | + | 0.46 | 0.463277 | 2.2 | 3.96E−05 | NM_182983 | 0.42 |
| chr21 | 34616788 | 34618980 | + | 0.29 | 0.282308 | 2.25 | 3.66E−05 | NM_207585 | 0.42 |
| chr3 | 1.34E+08 | 1.34E+08 | + | 0.75 | 0.100892 | 2.39 | 1.31E−06 | NM_016201 | 0.42 |
| chr9 | 1.19E+08 | 1.19E+08 | + | 2.04 | 0.008345 | 1.86 | 1.64E−08 | NM_198186 | 0.41 |
| chr18 | 56743151 | 56743384 | + | −0.54 | 0.162324 | 2.56 | 1.60E−09 | NR_024021 | 0.4 |
| chr9 | 46115227 | 46115417 | + | 0.48 | 0.150594 | 2.11 | 3.97E−05 | NR_103712 | 0.4 |
| chr3 | 1.73E+08 | 1.73E+08 | + | −2.54 | 0.044242 | 2.41 | 3.22E−05 | NM_014932 | 0.39 |
| chr3 | 1.73E+08 | 1.73E+08 | + | 0.17 | 0.379053 | 1.5 | 2.98E−07 | NM_014932 | 0.39 |
| chr16 | 27793865 | 27794030 | + | −0.17 | 0.344028 | 2.2 | 5.95E−05 | NM_144675 | 0.38 |
| chr3 | 1.53E+08 | 1.53E+08 | + | −0.68 | 0.134838 | 1.48 | 4.35E−07 | NM_002563 | 0.38 |
| chr3 | 1.53E+08 | 1.53E+08 | + | 0.04 | 0.529972 | 2.25 | 3.66E−05 | NM_002563 | 0.38 |
| chr7 | 55320652 | 55320780 | + | −0.81 | 0.006997 | 1.6 | 2.91E−08 | NR_047551 | 0.38 |
| chr9 | 1.246+08 | 1.24E+08 | + | 1.63 | 0.07633 | 2.52 | 7.56E−05 | NM_138709 | 0.38 |
| chr1 | 2.08E+08 | 2.08E+08 | + | 2.26 | 0.010657 | 1.67 | 1.97E−07 | NM_025179 | 0.37 |
| chr2 | 2.02E+08 | 2.02E+08 | + | 1.78 | 0.01992 | 2.36 | 5.33E−05 | NM_001142356 | 0.37 |
| chr3 | 29502406 | 29502534 | + | −0.06 | 0.44608 | 2.43 | 2.32E−08 | NM_001177712 | 0.37 |
| chr11 | 85251497 | 85251925 | + | 2.16 | 0.000802 | 1.5 | 7.44E−07 | NM_001142699 | 0.36 |
| chr16 | 726153 | 726367 | + | 1.78 | 0.01992 | 2.41 | 3.22E−05 | NM_003961 | 0.36 |
| chr18 | 25597547 | 25597712 | + | 1.04 | 0.085353 | 2.54 | 1.09E−03 | NM_001792 | 0.36 |
| chr18 | 25613369 | 25813506 | + | 0.78 | 0.201565 | 1.95 | 2.71E−05 | NM_001792 | 0.36 |
| chr18 | 25729930 | 25730338 | + | 0.22 | 0.252701 | 1.39 | 4.13E−08 | NM_001792 | 0.36 |
| chr18 | 26470533 | 26471078 | + | 2.57 | 0.00024 | 1.72 | 9.93E−09 | NM_001792 | 0.36 |
| chr2 | 12883536 | 12883741 | + | −0.21 | 0.309874 | 1.63 | 5.84E−05 | NR_036072 | 0.36 |
| chr22 | 39644794 | 39644930 | + | −1.54 | 0.103108 | 3.05 | 6.68E−06 | NM _002608 | 0.36 |
| chr22 | 39650793 | 39651509 | + | 1.29 | 5.83E−05 | 1.36 | 5.78E−12 | NM_002608 | 0.36 |
| chr3 | 1.12E+08 | 1.12E+08 | + | −0.06 | 0.44608 | 2.34 | 1.37E−05 | NM_199512 | 0.36 |
| chr8 | 1.45E+08 | 1.45E+08 | + | 3.54 | 0.000686 | 2.68 | 1.57E−05 | NM_015117 | 0.36 |
| chr9 | 80512297 | 80512736 | + | −0.28 | 0.310976 | 1.3 | 1.03E−05 | NM_00272 | 0.36 |
| chr9 | 1.27E+08 | 1.27E+08 | + | 1.88 | 0.000267 | 1.48 | 2.73E−06 | NR_038975 | 0.36 |
| chr9 | 1.34E+08 | 1.34E+08 | + | 0.58 | 0.142237 | 1.4 | 2.62E−08 | NM_033387 | 0.36 |
| chr2 | 1.66E+08 | 1.66E+08 | + | −0.54 | 0.261994 | 2.78 | 2.43E−10 | NM_001172173 | 0.35 |
| chr7 | 196138 | 156498 | + | 0.78 | 0.077498 | 1.98 | 6.53E−09 | NM_020223 | 0.35 |
| chr11 | 33497653 | 33498339 | + | 0.39 | 0.249424 | 1.88 | 8.33E−13 | NM_012194 | 0.34 |
| chr11 | 33502186 | 33502435 | + | 0.78 | 0.301098 | 1.92 | 4.25E−05 | NM_012194 | 0.34 |
| chr12 | 64291605 | 64292134 | + | 0.46 | 0.136027 | 1.38 | 4.02E−07 | NM_020762 | 0.34 |
| chr12 | 64303495 | 64303644 | + | −0.87 | 0.178044 | 2.25 | 3.66E−05 | NM_020762 | 0.34 |
| chr12 | 64311063 | 64312478 | + | 0.74 | 0.006402 | 1.34 | 1.59E−12 | NM_020762 | 0.34 |
| chr12 | 64338601 | 64338763 | + | 1.23 | 0.001123 | 3.46 | 4.08E−08 | NM_020762 | 0.34 |
| chr2 | 70985550 | 70985924 | + | −0.2 | 0.353044 | 1.9 | 2.20E−07 | NM_001185054 | 0.34 |
| chr10 | 1.03E+08 | 1.03E+08 | + | 1.19 | 0.001665 | 1.43 | 1.38E−10 | NM_018121 | 0.33 |
| chr12 | 1.23E+08 | 1.23E+08 | + | 0.35 | 0.325791 | 2.51 | 1.16E−05 | NM_003959 | 0.33 |
| chr13 | 44336319 | 44336853 | + | 2.23 | 2.13E−06 | 3.01 | 2.28E−14 | NM_001242863 | 0.33 |
| chr13 | 44337500 | 44337834 | + | 0.46 | 0.286689 | 1.93 | 6.73E−09 | NM_001242863 | 0.33 |
| chr13 | 44338512 | 44339608 | + | 1.81 | 6.81E−05 | 2.35 | 1.71E−21 | NM_001242863 | 0.33 |
| chr13 | 44341224 | 44341935 | + | 2.7 | 2.75E−08 | 1.83 | 1.81E−14 | NM_001242863 | 0.33 |
| chr13 | 44352470 | 44352671 | + | 3.04 | 0.007085 | 3.58 | 7.18E−09 | NM_001242863 | 0.33 |
| chr2 | 65641494 | 65641893 | + | 0.94 | 0.037428 | 1.58 | 3.95E−07 | NM_181784 | 0.33 |
| chr8 | 1.19E+08 | 1.19E+08 | + | −0.67 | 0.128489 | 1.97 | 2.96E−06 | NM_000127 | 0.33 |
| chr10 | 95854338 | 95854466 | + | −0.54 | 0.261994 | 3.4 | 1.33E−05 | NM_001165979 | 0.32 |
| chr10 | 1E+08 | 1E+08 | + | 2.78 | 0.017487 | 1.68 | 6.15E−05 | NM_032211 | 0.32 |
| chr14 | 1E+08 | 1E+08 | + | 2.26 | 0.065139 | 2.52 | 7.56E−05 | NM_001144995 | 0.32 |
| chr15 | 35475360 | 35475517 | + | −0.96 | 0.18457 | 4.96 | 2.25E−09 | NR_003144 | 0.32 |
| chr22 | 38662786 | 38663074 | + | 0.68 | 0.090078 | 1.41 | 8.54E−05 | NM_001195071 | 0.32 |
| chr6 | 17698540 | 17698774 | + | −0.42 | 0.23405 | 1.84 | 4.25E−05 | NM_001278210 | 0.32 |
| chr6 | 1.49E+08 | 1.49E+08 | + | −0.51 | 0.00454 | 2.41 | 3.22E−05 | NM_005715 | 0.32 |
| chr8 | 1.26E+08 | 1.26E+08 | + | −0.37 | 0.139459 | 1.73 | 2.08E−10 | NM_005005 | 0.32 |
| chr11 | 93868890 | 93869064 | + | −0.87 | 0.178044 | 2.24 | 9.42E−06 | NM_015368 | 0.31 |
| chr13 | 43996261 | 43996733 | + | 0.46 | 0.197118 | 1.55 | 1.36E−06 | NM_001127615 | 0.31 |
| chr13 | 44146720 | 44147097 | + | 2.52 | 0.000366 | 3.05 | 2.29E−13 | NM_001127615 | 0.31 |
| chr13 | 44205448 | 44205587 | + | −0.13 | 0.422029 | 4.27 | 6.94E−06 | NM_001127615 | 0.31 |
| chr4 | 5180013 | 5180217 | + | 0.04 | 0.487929 | 2.24 | 9.42E−06 | NM_018401 | 0.31 |
| chrX | 50388208 | 50388386 | + | 1.57 | 0.041179 | 2.93 | 2.01E−05 | NM_020717 | 0.31 |
| chr12 | 43376666 | 43376912 | + | −1.28 | 0.107269 | 2.39 | 1.31E−06 | NM_153026 | 0.3 |
| chr17 | 950547 | 950858 | + | 0.74 | 0.054754 | 1.97 | 5.77E−08 | NM_001256847 | 0.3 |
| chr18 | 24365392 | 24365527 | + | 0.01 | 0.509 | 1.8 | 6.58E−05 | NM_004028 | 0.3 |
| chr6 | 26022824 | 26022952 | + | 1.93 | 0.000128 | 2.81 | 5.99E−05 | NM_003538 | 0.3 |
| chr9 | 82310786 | 82311067 | + | 0.72 | 0.192023 | 1.98 | 7.12E−06 | NM_007005 | 0.3 |
| chr1 | 43998889 | 43999193 | + | 0.68 | 0.182687 | 2.11 | 1.12E−06 | NM_002840 | 0.29 |
| chr1 | 2.21E+08 | 2.21E+08 | + | −0.13 | 0.39179 | 1.85 | 5.28E−07 | NM_022746 | 0.29 |
| chr11 | 68150884 | 68151023 | + | 1.04 | 0.114476 | 2.83 | 3.17E−06 | NM_002335 | 0.29 |
| chr12 | 77625139 | 77625382 | + | −0.22 | 0.38435 | 3.81 | 1.18E−10 | NM_203394 | 0.29 |
| chr2 | 7077412 | 7077553 | + | 0 | 0.593079 | 2.41 | 3.22E−05 | NR_033997 | 0.29 |
| chr2 | 1.92E+08 | 1.92E+08 | + | −0.36 | 0.928622 | 2.13 | 6.02E−07 | NM_001254738 | 0.29 |
| chr3 | 20659943 | 20660111 | + | 3.28 | 0.002807 | 2.2 | 1.53E−05 | NM_001199251 | 0.29 |
| chr3 | 20679118 | 20679385 | + | 2.26 | 0.010657 | 2.65 | 2.30E−07 | NM_001199251 | 0.29 |
| chr3 | 20689915 | 20690075 | + | 0.46 | 0.404925 | 3.52 | 4.18E−06 | NM_001199251 | 0.29 |
| chr3 | 20711116 | 20712204 | + | 1.9 | 1.66E−07 | 2.22 | 1.80E−20 | NM_001199251 | 0.29 |
| chr3 | 20784774 | 20784902 | + | 0.81 | 0.93262 | 4.27 | 6.94E−06 | NM_001199251 | 0.29 |
| chr3 | 20818080 | 20818222 | + | 0.23 | 0.02581 | 2.73 | 9.25E−06 | NM_001199251 | 0.29 |
| chr3 | 1.7E+08 | 1.7E+08 | + | −0.99 | 0.039705 | 1.85 | 9.30E−05 | NM_001248008 | 0.29 |
| chrX | 33109524 | 33110058 | + | 0.21 | 0.336355 | 1.34 | 3.23E−08 | NM_004010 | 0.29 |
| chrX | 1.4E+08 | 1.4E+08 | + | 2.34 | 1.66E−07 | 2.29 | 2.74E−09 | NR_037183 | 0.29 |
| chr10 | 1.34E+08 | 1.34E+08 | + | −0.54 | 0.22977 | 1.9 | 1.26E−06 | NM_005539 | 0.28 |
| chr8 | 20041831 | 20042126 | + | 0.92 | 0.063595 | 1.39 | 4.86E−05 | NM_003053 | 0.28 |
| chr8 | 1.31E+08 | 1.31E+08 | + | 1.96 | 5.10E−09 | 1.52 | 5.75E−09 | NR_045386 | 0.28 |
| chrX | 39719416 | 39719681 | + | −0.54 | 0.3033 | 2.17 | 1.15E−07 | NM_001123385 | 0.28 |
| chr1 | 2.04E+08 | 2.04E+08 | + | 1.95 | 3.75E−05 | 1.36 | 9.08E−07 | NM_002646 | 0.27 |
| chr11 | 73229622 | 73229764 | + | 3.16 | 0.004461 | 3.52 | 4.13E−06 | NM_015159 | 0.27 |
| chr11 | 73246550 | 73248704 | + | −0.35 | 0.243715 | 2.29 | 2.24E−05 | NM_015159 | 0.27 |
| chr17 | 17614363 | 17614539 | + | 0.68 | 0.126482 | 2.24 | 9.42E−06 | NM_030665 | 0.27 |
| chr17 | 17628702 | 17628834 | + | 0.87 | 0.110781 | 3.1 | 3.83E−06 | NM_030665 | 0.27 |
| chr14 | 90877225 | 90877483 | + | 0.46 | 0.158627 | 3.58 | 2.32E−11 | NM_006888 | 0.26 |
| chr15 | 30178079 | 30178257 | + | 0.37 | 0.283356 | 3.05 | 2.06E−07 | NM_175610 | 0.26 |
| chr18 | 2223598 | 2223968 | + | 0.53 | 0.18278 | 1.78 | 2.17E−10 | NM_022840 | 0.26 |
| chr8 | 23896972 | 23897222 | + | −0.68 | 0.134838 | 2.1 | 4.26E−06 | NM_003155 | 0.26 |
| chr9 | 91991923 | 91992057 | + | 0.46 | 0.463277 | 3.52 | 4.18E−06 | NM_024077 | 0.26 |
| chr10 | 88616192 | 88616869 | + | 0.08 | 0.42904 | 1.78 | 4.41E−09 | NM_004329 | 0.25 |
| chr11 | 63825957 | 63826106 | + | −1.39 | 0.038282 | 2.83 | 3.178−06 | NM_013280 | 0.25 |
| chr20 | 51670842 | 51670979 | + | −1.28 | 0.039667 | 2.64 | 2.45E−06 | NM_173485 | 0.25 |
| chr19 | 5310392 | 5310591 | + | −0.35 | 0.243715 | 2.03 | 1.09E−05 | NM_002850 | 0.24 |
| chr19 | 14360631 | 14360841 | + | −0.17 | 0.344028 | 1.38 | 7.74E−05 | NM_001008701 | 0.24 |
| chr20 | 51768821 | 51769012 | + | 0.16 | 0.427697 | 1.74 | 6.40E−05 | NM_001193421 | 0.24 |
| chr21 | 35350428 | 35350593 | + | 0.04 | 0.492338 | 2.63 | 2.67E−05 | NR_038883 | 0.24 |
| chr5 | 73727131 | 73727292 | + | 0.85 | 0.081853 | 2.01 | 4.17E−05 | NM_003633 | 0.24 |
| chr5 | 73930549 | 73932593 | + | 1.65 | 1.36E−11 | 1.52 | 8.71E−26 | NM_003633 | 0.24 |
| chr6 | 48056428 | 48056616 | + | 1.53 | 0.01294 | 2.15 | 9.64E−05 | NM_001013732 | 0.24 |
| chr1 | 24335543 | 24338292 | + | 0.72 | 0.097279 | 1.4 | 2.80E−07 | NR_034035 | 0.23 |
| chr10 | 1.25E+08 | 1.25E+08 | + | 2.26 | 0.065139 | 2.73 | 9.25E−06 | NM_004725 | 0.23 |
| chr2 | 2.32E+08 | 2.32E+08 | + | 0.19 | 0.465306 | 3.27 | 4.18E−05 | NR_034059 | 0.23 |
| chr20 | 50619904 | 50620074 | + | −0.54 | 0.329466 | 4.16 | 1.02E−09 | NM_199427 | 0.23 |
| chr20 | 50907812 | 50907989 | + | 1.04 | 0.325105 | 2.11 | 3.97E−05 | NM_199427 | 0.23 |
| chr20 | 51078245 | 51078510 | + | 0.19 | 0.465306 | 1.63 | 9.73E−06 | NM_199427 | 0.23 |
| chr20 | 51115117 | 51117337 | + | 1.44 | 3.81E−09 | 1.43 | 1.41E−29 | NM_199427 | 0.23 |
| chr3 | 1.54E+08 | 1.54E+08 | + | −1.36 | 0.791388 | 2.01 | 4.17E−05 | NM_001251962 | 0.23 |
| chr1 | 954406 | 954593 | + | −0.22 | 0.35557 | 2.38 | 8.30E−06 | NM_198576 | 0.22 |
| chr1 | 2.06E+08 | 2.06E+08 | + | 0.78 | 0.077498 | 1.82 | 1.95E−06 | NM_001123168 | 0.22 |
| chr11 | 16932012 | 16932827 | + | 1.29 | 0.006935 | 1.91 | 5.30E−13 | NM_175058 | 0.22 |
| chr16 | 73948502 | 73948683 | + | 3.36 | 8.86E−06 | 1.37 | 0.00236 | NM_002811 | 0.22 |
| chr17 | 2137082 | 2137379 | + | 0.71 | 0.070162 | 1.34 | 2.25E−05 | NM_001256828 | 0.22 |
| chr19 | 17202119 | 17202422 | + | 0.16 | 0.32225 | 2.04 | 1.48E−08 | NM_001130065 | 0.22 |
| chr5 | 56136138 | 56136266 | + | 0.46 | 0.404925 | 3.58 | 2.33E−06 | NM_005921 | 0.22 |
| chr8 | 1.24E+08 | 1.24E+08 | + | 1.78 | 0.149771 | 1.34 | 1.42E−05 | NM_024295 | 0.22 |
| chr8 | 1.42E+08 | 1.42E+08 | + | 0.71 | 0.005577 | 1.94 | 1.22E−12 | NM_001164823 | 0.22 |
| chr1 | 43941605 | 43941830 | + | −1.13 | 0.188905 | 1.82 | 1.95E−06 | NM_001190880 | 0.21 |
| chr1 | 1.58E+08 | 1.58E+08 | + | 1.92 | 0.035602 | 2.31 | 8.78E−05 | NM_018240 | 0.21 |
| chr1 | 1.58E+08 | 1.58E+08 | + | 0.18 | 0.360591 | 1.72 | 3.20E−07 | NM_018240 | 0.21 |
| chr13 | 91369397 | 91369807 | + | 0.64 | 0.115893 | 1.44 | 2.32E−05 | NR_027039 | 0.21 |
| chr17 | 45982250 | 45982748 | + | 1.26 | 0.011922 | 1.46 | 6.31E−06 | NM_003110 | 0.21 |
| chr4 | 1.6E+08 | 1.6E+08 | + | 1.63 | 0.016149 | 2.3 | 9.22E−07 | NM_014247 | 0.21 |
| chr4 | 1.61E+08 | 1.61E+08 | + | 2.46 | 0.042327 | 3.34 | 2.36E−05 | NM_014247 | 0.21 |
| chr5 | 1.72E+08 | 1.72E+08 | + | 0.78 | 0.25643 | 1.46 | 1.58E−06 | NM_001017995 | 0.21 |
| chr9 | 1.07E+08 | 1.07E+08 | + | 0.9 | 0.001929 | 1.37 | 4.39E−05 | NM_001042551 | 0.21 |
| chr9 | 1.07E+08 | 1.07E+08 | + | 0.87 | 0.210905 | 3.2 | 1.25E−06 | NM_001042551 | 0.21 |
| chr9 | 1.07E+08 | 1.07E+08 | + | 1.36 | 0.044821 | 1.46 | 4.04E−05 | NM_001042550 | 0.21 |
| chr9 | 1.07E+08 | 1.07E+08 | + | 1.16 | 0.08465 | 3.25 | 7.10E−07 | NM_001042550 | 0.21 |
| chr1 | 58875361 | 58876232 | + | 0.68 | 0.090078 | 1.45 | 9.60E−09 | NM_145243 | 0.2 |
| chr15 | 57550260 | 27550529 | + | 1.68 | 0.006504 | 1.95 | 1.12E−05 | NM_207040 | 0.2 |
| chr17 | 2511784 | 2513068 | + | 1.42 | 1.22E−05 | 1.39 | 1.40E−12 | NM_000430 | 0.2 |
| chr17 | 20932205 | 20932373 | + | 2.1 | 0.001188 | 2.41 | 3.22E−05 | NM_015276 | 0.2 |
| chr18 | 56589949 | 56590098 | + | −0.41 | 0.166483 | 3.15 | 2.19E−06 | NM_018181 | 0.2 |
| chr11 | 64611591 | 64611765 | + | −1.03 | 0.107039 | 2.68 | 1.57E−05 | NM_017525 | 0.19 |
| chr15 | 68060682 | 58060979 | + | 0.04 | 0.504309 | 1.46 | 4.04E−05 | NM_001258024 | 0.19 |
| chr17 | 1286044 | 1286197 | + | 0.26 | 0.418658 | 1.68 | 6.15E−05 | NM_006761 | 0.19 |
| chr17 | 28244948 | 28245077 | + | 1.04 | 0.325105 | 2.81 | 5.99E−05 | NM_001145053 | 0.19 |
| chr19 | 34050055 | 34050355 | + | −0.54 | 0.3033 | 1.44 | 3.65E−06 | NM_000285 | 0.19 |
| chr6 | 1.35E+08 | 1.35E+08 | + | 0.46 | 0.308555 | 2.31 | 1.69E−09 | NR_038219 | 0.19 |
| chr9 | 1.23E+08 | 1.23E+08 | + | 0.13 | 0.464479 | 2.58 | 4.50E−05 | NR_073557 | 0.19 |
| chr1 | 27891918 | 27892088 | + | 0.9 | 0.083608 | 3.12 | 6.79E−08 | NM_001029882 | 0.18 |
| chr14 | 59788968 | 59789229 | + | 1.11 | 0.036985 | 3.54 | 1.28E−08 | NM_014992 | 0.18 |
| chr18 | 47325795 | 47325925 | + | 1.57 | 0.041179 | 2.46 | 1.94E−05 | NM_006111 | 0.18 |
| chr20 | 56236365 | 56236537 | + | 1.46 | 0.00118 | 2.36 | 5.33E−05 | NM_001255976 | 0.18 |
| chr20 | 56241983 | 56242159 | + | 4.21 | 5.50E−06 | 1.6 | 0.00054 | NM_001255976 | 0.18 |
| chr20 | 62091442 | 62091654 | + | 0.51 | 0.155661 | 1.8 | 6.58E−05 | NM_172107 | 0.18 |
| chr20 | 62093991 | 62094204 | + | 1.11 | 0.036985 | 2.75 | 4.74E−08 | NM_172107 | 0.18 |
| chr4 | 1.24E+08 | 1.24E+08 | + | 1.9 | 1.16E−18 | 2.24 | 1.03E−23 | NM_001258038 | 0.18 |
| chr4 | 1.24E+08 | 1.24E+08 | + | 2.08 | 4.82E−20 | 1.42 | 1.23E−07 | NM_001258038 | 0.18 |
| chr9 | 45045301 | 45045495 | + | 0.46 | 0.334119 | 1.8 | 6.58E−05 | NR_027421 | 0.18 |
| chrX | 1.3E+08 | 1.3E+08 | + | 2.12 | 0.004281 | 2.01 | 4.17E−05 | NR_027141 | 0.18 |
| chrX | 1.3E+08 | 1.3E+08 | + | 2.98 | 0.000217 | 1.64 | 2.60E−06 | NR_027141 | 0.18 |
| chrX | 1.3E+08 | 1.3E+08 | + | 1.6 | 3.06E−11 | 1.95 | 5.51E−33 | NR_027141 | 0.18 |
| chrX | 1.3E+08 | 1.3E+08 | + | 0.04 | 0.492338 | 1.42 | 5.28E−06 | NR_027141 | 0.18 |
| chr1 | 16179816 | 16179944 | + | 0.04 | 0.504309 | 3.1 | 3.83E−06 | NR_024279 | 0.17 |
| chr10 | 31261588 | 31251817 | + | −0.34 | 0.258417 | 1.84 | 1.76E−05 | NM_001143769 | 0.17 |
| chr12 | 1.05E+08 | 1.05E+08 | + | −0.54 | 0.281197 | 3.4 | 1.33E−05 | NM_018413 | 0.17 |
| chr12 | 1.05E+08 | 1.05E+08 | + | −0.22 | 0.38435 | 1.81 | 1.12E−05 | NM_018413 | 0.17 |
| chr16 | 2232770 | 2232941 | + | −2.54 | 0.044242 | 3.1 | 3.83E−06 | NM_020764 | 0.17 |
| chr2 | 12559899 | 12560135 | + | 0.23 | 0.317498 | 1.58 | 3.45E−05 | NM_021643 | 0.17 |
| chr20 | 59860902 | 59861030 | + | −0.54 | 0.245002 | 2.93 | 2.01E−05 | NM_001794 | 0.17 |
| chr20 | 60136453 | 60136623 | + | −1.13 | 0.065395 | 1.91 | 1.75E−05 | NM_001252339 | 0.17 |
| chr4 | 56964871 | 56965203 | + | −0.22 | 0.35557 | 1.65 | 9.30E−05 | NM_020722 | 0.17 |
| chr1 | 1.5E+08 | 1.5E+08 | + | 0.36 | 0.303275 | 3.27 | 4.18E−05 | NM_001136479 | 0.16 |
| chr11 | 46959447 | 46959748 | + | 0.97 | 0.064254 | 1.51 | 1.88E−05 | NM_001003678 | 0.16 |
| chr15 | 59018593 | 59018761 | + | 2.26 | 0.065139 | 3.68 | 7.23E−07 | NM_001110 | 0.16 |
| chr15 | 80711486 | 80711619 | + | 2.16 | 0.016043 | 2.81 | 5.99E−05 | NM_014862 | 0.16 |
| chr17 | 631552 | 631722 | + | 0.19 | 0.465306 | 1.8 | 6.58E−05 | NM_024792 | 0.16 |
| chr18 | 60460282 | 60460410 | + | 0.46 | 0.308555 | 2.78 | 5.42E−06 | NM_194449 | 0.16 |
| chr18 | 60468692 | 60468831 | + | −0.71 | 0.148471 | 2.07 | 6.37E−05 | NM_194449 | 0.16 |
| chr2 | 98222338 | 98222803 | + | 1.46 | 0.018075 | 1.5 | 1.86E−06 | NM_025190 | 0.16 |
| chr5 | 15069959 | 15070110 | + | 1.78 | 0.052385 | 2.68 | 1.57E−05 | NM_054027 | 0.16 |
| chr1 | 7434513 | 7434731 | + | 2.03 | 1.02E−05 | 1.4 | 0.00035 | NM_004781 | 0.15 |
| chr11 | 88027196 | 88027324 | + | 0.81 | 0.005384 | 3.05 | 6.68E−06 | NM_001814 | 0.15 |
| chr11 | 89981373 | 89981533 | + | −2.42 | 0.002298 | 2.31 | 8.78E−05 | NM_001144073 | 0.15 |
| chr19 | 49116753 | 49116978 | + | 0.75 | 0.100892 | 1.9 | 1.26E−06 | NM_0177708 | 0.15 |
| chr19 | 49591785 | 49591916 | + | 1.38 | 0.025068 | 2.58 | 4.50E−05 | NM_003089 | 0.15 |
| chr20 | 40021741 | 40021872 | + | −1.87 | 0.054231 | 3.63 | 1.30E−08 | NM_052846 | 0.15 |
| chr3 | 1.28E+08 | 1.28E+08 | + | 0.04 | 0.529972 | 2.36 | 5.33E−05 | NM_207335 | 0.15 |
| chr5 | 67702765 | 67702894 | + | −1.22 | 0.064934 | 3.34 | 2.36E−05 | NM_001242466 | 0.15 |
| chr8 | 26059915 | 26060074 | + | 2.63 | 0.027294 | 2.51 | 1.16E−05 | NM_002717 | 0.15 |
| chr9 | 2414499 | 2414664 | + | 0.19 | 0.465306 | 2.78 | 5.42E−06 | NM_003383 | 0.15 |
| chrX | 46455295 | 46455502 | + | 1.68 | 0.002665 | 1.88 | 2.74E−05 | NM_019886 | 0.15 |
| chr16 | 67598952 | 67599211 | + | 0.37 | 0.283356 | 1.46 | 2.53E−05 | NM_006565 | 0.14 |
| chr2 | 64913142 | 64913309 | + | 0.46 | 0.365076 | 2.92 | 1.07E−06 | NM_014755 | 0.14 |
| chr20 | 25605631 | 25605770 | + | 1.36 | 0.044821 | 1.84 | 1.75E−05 | NM_152667 | 0.14 |
| chr3 | 39069341 | 39069929 | + | 1.26 | 0.000269 | 1.89 | 2.30E−13 | NM_020839 | 0.14 |
| chr6 | 1.51E+08 | 1.51E+08 | + | −0.96 | 0.004213 | 1.82 | 3.38E−10 | NM_030949 | 0.14 |
| chr1 | 21491558 | 21491724 | + | 1.04 | 0.156144 | 2.1 | 4.28E−06 | NM_001198803 | 0.13 |
| chr1 | 87584227 | 87584355 | + | 1.68 | 0.028759 | 2.88 | 3.48E−05 | NR_026989 | 0.13 |
| chr1 | 1.81E+08 | 1.81E+08 | + | 0.11 | 0.462988 | 1.44 | 1.46E−05 | NM_005819 | 0.13 |
| chr1 | 2.43E+08 | 2.43E+08 | + | 0.26 | 0.418658 | 3.09 | 1.18E−07 | NR_029401 | 0.13 |
| chr1 | 2.43E+08 | 2.43E+08 | + | 1.63 | 0.07633 | 2.01 | 4.17E−05 | NR_029401 | 0.13 |
| chr12 | 77075107 | 77075235 | + | 1.26 | 0.156387 | 3.46 | 7.46E−06 | NM+015336 | 0.13 |
| chr19 | 48162101 | 48162258 | + | 0.54 | 0.204578 | 1.96 | 6.59E−05 | NM_015711 | 0.13 |
| chr22 | 37994944 | 37995624 | + | 1.13 | 0.083706 | 1.51 | 5.97E−10 | NM_013365 | 0.13 |
| chr5 | 1.53E+08 | 1.53E+08 | + | 0.87 | 0.062557 | 2.16 | 2.45E−17 | NM_001258022 | 0.13 |
| chr5 | 1.53E+08 | 1.53E+08 | + | 1.46 | 0.109973 | 3.63 | 1.30E−06 | NM_001258022 | 0.13 |
| chr5 | 1.53E+08 | 1.53E+08 | + | 0.76 | 3.05E−06 | 1.34 | 1.63E−18 | NM_001258022 | 0.13 |
| chr5 | 1.53E+08 | 1.53E+08 | + | 2.7 | 5.57E−06 | 2.2 | 1.32E−13 | NM_001258022 | 0.13 |
| chr5 | 1.53E+08 | 1.53E+08 | + | 0.98 | 0.049001 | 3.05 | 3.33E−22 | NM_001258022 | 0.13 |
| chr5 | 1.53E+08 | 1.53E+08 | + | −0.36 | 0.926622 | 4.27 | 6.94E−06 | NM_001258022 | 0.13 |
| chr5 | 1.53E+08 | 1.53E+08 | + | 1.06 | 0.000284 | 1.45 | 2.16E−15 | NM_001258022 | 0.13 |
| chr5 | 1.53E+08 | 1.53E+08 | + | 3.92 | 6.26E−05 | 3 | 2.07E−11 | NM_001258022 | 0.13 |
| chr5 | 1.53E+08 | 1.53E+08 | + | 2.4 | 7.49E−13 | 1.99 | 3.90E−27 | NM_001258022 | 0.13 |
| chr5 | 1.53E+08 | 1.53E+08 | + | −0.54 | 0.329466 | 4.34 | 3.77E−06 | NM_001258022 | 0.13 |
| chr5 | 1.53E+08 | 1.53E+08 | + | −0.96 | 0.18457 | 3.68 | 7.23E−07 | NM_001258022 | 0.13 |
| chr6 | 87921887 | 87922118 | + | −0.18 | 0.381896 | 1.95 | 1.12E−05 | NM_015021 | 0.13 |
| chr6 | 1.66E+08 | 1.66E+08 | + | 0.57 | 0.230388 | 1.36 | 6.99E−05 | NM_144980 | 0.13 |
| chr1 | 21446824 | 21447079 | + | 0.78 | 0.077498 | 1.44 | 2.32E−05 | NM_003760 | 0.12 |
| chr15 | 81217868 | 81218519 | + | −0.75 | 0.044073 | 2.01 | 5.42E−11 | NM_015154 | 0.12 |
| chr16 | 89539456 | 89539585 | + | 0.26 | 0.418658 | 2.15 | 9.64E−05 | NM_013275 | 0.12 |
| chr18 | 42268830 | 42268964 | + | 0.46 | 0.334119 | 1.74 | 6.40E−05 | NM_015559 | 0.12 |
| chr19 | 39356017 | 39356256 | + | −0.96 | 0.104912 | 1.7 | 9.76E−05 | NM_198445 | 0.12 |
| chr19 | 54613297 | 54613451 | + | 0.46 | 0.158627 | 2.2 | 1.53E−05 | NM_015629 | 0.12 |
| chr22 | 36341267 | 36341395 | + | −0.32 | 0.326824 | 4.4 | 2.04E−06 | NM_001082578 | 0.12 |
| chr3 | 55356599 | 55358782 | + | 1.46 | 0.109973 | 2.03 | 1.19E−06 | NM_001256105 | 0.12 |
| chr3 | 55356599 | 55358782 | + | 1.04 | 0.003074 | 1.8 | 4.63E−17 | NM_001256105 | 0.12 |
| chr3 | 55457392 | 55457817 | + | 0.46 | 0.176457 | 1.61 | 6.61E−07 | NM_001256105 | 0.12 |
| chr3 | 55458323 | 55459016 | + | 0.36 | 0/231846 | 4.76 | 1.30E−35 | NM_001256105 | 0.12 |
| chr3 | 55460115 | 55462031 | + | 0.57 | 0.034711 | 1.94 | 1.18E−29 | NM_001256105 | 0.12 |
| chr3 | 55500856 | 55501335 | + | 0.68 | 0.280156 | 1.63 | 7.60E−08 | NM_001256105 | 0.12 |
| chr5 | 1.53E+08 | 1.53E+08 | + | −1.35 | 0.06335 | 1.84 | 6.01E−08 | NM_005927 | 0.12 |
| chr7 | 1.54E+08 | 1.54E+08 | + | 0.81 | 0.93262 | 2.63 | 2.67E−05 | NR_024477 | 0.12 |
| chr9 | 7983611 | 7983880 | + | 0.29 | 0.337406 | 1.52 | 7.61E−05 | NM_033428 | 0.12 |
| chr9 | 27086846 | 27087018 | + | −0.78 | 0.049269 | 1.96 | 6.59E−05 | NM_000459 | 0.12 |
| chr11 | 1.11E+08 | 1.11E+08 | + | 0.96 | 0.084864 | 1.31 | 1.25E−07 | NR_034154 | 0.11 |
| chr11 | 1.11E+08 | 1.11E+08 | + | 1.34 | 0.000868 | 1.95 | 9.04E−08 | NR_034154 | 0.11 |
| chr11 | 1.11E+08 | 1.11E+08 | + | 0.64 | 0.115893 | 1.34 | 1.42E−05 | NR_034154 | 0.11 |
| chr11 | 1.11E+08 | 1.11E+08 | + | 0.56 | 0.077732 | 1.68 | 1.67E−12 | NR_034154 | 0.11 |
| chr17 | 3570198 | 3570450 | + | 1.1 | 0.028425 | 1.64 | 1.54E−05 | NM_014604 | 0.11 |
| chr2 | 1.1E+08 | 1.1E+08 | + | 1.04 | 0.06437 | 1.7 | 2.56E−05 | NM_144710 | 0.11 |
| chr2 | 1.1E+08 | 1.1E+08 | + | 0.01 | 0.504539 | 1.87 | 4.60E−17 | NM_144710 | 0.11 |
| chr20 | 47385126 | 47385418 | + | 1.13 | 0.010049 | 1.53 | 3.19E−06 | NM_020820 | 0.11 |
| chr4 | 88437065 | 88437918 | + | 0.33 | 0.119205 | 1.4 | 4.08E−10 | NM_004684 | 0.11 |
| chr5 | 88172041 | 88172169 | + | 1.04 | 0.325105 | 2.26 | 1.64E−07 | NM_001193350 | 0.11 |
| chr9 | 45609849 | 45610441 | + | −0.54 | 0.19189 | 1.46 | 2.51E−06 | NR_024060 | 0.11 |
| chr9 | 1.28E+08 | 1.28E+08 | + | 0.46 | 0.404925 | 2.16 | 2.47E−05 | NM_001006617 | 0.11 |
| chr1 | 14117179 | 14117511 | + | 0.65 | 0.083141 | 1.44 | 1.46E−05 | NM_001007257 | 0.1 |
| chr10 | 82241644 | 82242201 | + | 1.7 | 0.000463 | 1.3 | 8.05E−05 | NM_001128309 | 0.1 |
| chr11 | 63914872 | 63915402 | + | −0.25 | 0.332095 | 1.9 | 2.20E−07 | NM_014067 | 0.1 |
| chr18 | 52891566 | 52891861 | + | 0.67 | 0.062848 | 1.44 | 9.18E−06 | NM_001243236 | 0.1 |
| chr18 | 52997517 | 52997653 | + | 0.93 | 0.048668 | 2.01 | 4.17E−05 | NM_001243235 | 0.1 |
| chr18 | 53028093 | 53028270 | + | 1.46 | 0.032155 | 1.97 | 2.96E−06 | NM_001243235 | 0.1 |
| chr18 | 53199411 | 53199573 | + | 0.28 | 0.418658 | 2.38 | 8.30E−06 | NM_001243231 | 0.1 |
| chr2 | 11594473 | 11594661 | + | 4.21 | 5.50E−06 | 1.68 | 0.000236 | NR_003095 | 0.1 |
| chr5 | 1.57E +08 | 1.57E+08 | + | 0.19 | 0.356801 | 1.85 | 3.03E−06 | NM_033274 | 0.1 |
| chr9 | 1.27E+08 | 1.27E+08 | + | −1.39 | 0.006059 | 2.46 | 1.94E−05 | NM_002799 | 0.1 |
| chr9 | 1.345+08 | 1.34E+08 | + | 1.46 | 0.018075 | 1.88 | 1.43E−07 | NR_033701 | 0.1 |
| chr12 | 22925635 | 22925784 | + | 2.26 | 0.065139 | 2.15 | 9.64E−05 | NM_0186385 | 0.09 |
| chr15 | 41406286 | 41406414 | + | 0.78 | 0.201565 | 2.06 | 1.15E−06 | NM_017553 | 0.09 |
| chr16 | 85598720 | 85599221 | + | 1.26 | 1.16E−05 | 1.51 | 2.03E−07 | NM_001134473 | 0.09 |
| chr18 | 693503 | 693735 | + | 0.57 | 0.250388 | 1.73 | 1.68E−05 | NM_202758 | 0.09 |
| chr3 | 1.25E+08 | 1.25E+08 | + | 1.17 | 0.027729 | 1.61 | 2.315−05 | NM_003794 | 0.09 |
| chr9 | 1.1E+08 | 1.1E+08 | + | 2.4 | 0.000845 | 2.31 | 8.78E−05 | NM_001244713 | 0.09 |
| chr1 | 38462438 | 38482676 | + | 2.92 | 1.15E−05 | 2.12 | 1.03E−05 | NM_006802 | 0.08 |
| chr12 | 1.24E+08 | 1.24E+08 | + | 0.87 | 0.000738 | 1.37 | 6.27E−06 | NM_001270434 | 0.08 |
| chr17 | 17387773 | 17387901 | + | 1.46 | 0.032155 | 2.63 | 2.67E−05 | NM_001270434 | 0.08 |
| chr18 | 3360281 | 3360622 | + | −0.31 | 0.198324 | 1.43 | 3.36E−07 | NM_174886 | 0.08 |
| chr2 | 56029291 | 56030156 | + | 0.3 | 0.192847 | 1.35 | 2.41E−09 | NM_033109 | 0.08 |
| chr2 | 56032718 | 56033331 | + | 2.12 | 4.01E−10 | 1.38 | 2.26E−09 | NM_033109 | 0.08 |
| chr4 | 61689874 | 61690013 | + | −1.13 | 0.188905 | 3.46 | 7.46E−06 | NM_015236 | 0.08 |
| chr4 | 80555530 | 80555666 | + | 2.36 | 0.007037 | 2.07 | 6.37E−05 | NM_038826 | 0.08 |
| chr9 | 1.39E+08 | 1.39E+08 | + | −0.46 | 0.166558 | 2.83 | 3.17E−08 | NM_001080849 | 0.08 |
| chr1 | 1.81E+08 | 1.81E+08 | + | 0.53 | 0.18278 | 1.76 | 7.78E−10 | NM_032360 | 0.07 |
| chr1 | 2.03E+08 | 2.03E+08 | + | 0.54 | 0.204578 | 1.32 | 4.59E−06 | NM_001197131 | 0.07 |
| chr1 | 2.32E+08 | 2.32E+08 | + | −0.23 | 0.252632 | 2.43 | 1.26E−07 | NM_001164549 | 0.07 |
| chr10 | 97099932 | 97100073 | + | −1.54 | 0.060085 | 2.01 | 4.17E−05 | NM_020992 | 0.07 |
| chr11 | 36262179 | 36262783 | + | −1.01 | 0.006775 | 1.59 | 1.85E−08 | NM_001101653 | 0.07 |
| chr11 | 67157991 | 67158339 | + | 2.46 | 0.000557 | 1.48 | 1.09E−05 | NM_004584 | 0.07 |
| chr3 | 39002898 | 39003141 | + | 1.04 | 0.017239 | 2.65 | 2.30E−07 | NM_014139 | 0.07 |
| chr5 | 32056159 | 32056672 | + | 0.02 | 0.486295 | 1.44 | 5.81E−07 | NM_022130 | 0.07 |
| chr5 | 32145928 | 32146372 | + | 1.13 | 0.012899 | 1.34 | 6.30E−05 | NM_022130 | 0.07 |
| chr7 | 5481662 | 5481790 | + | 0.82 | 0.10778 | 1.74 | 6.40E−05 | NM_001080495 | 0.07 |
| chr9 | 1.2E+08 | 1.2E+08 | + | −0.22 | 0.38435 | 1.85 | 7.28E−06 | NM_012210 | 0.07 |
| chr9 | 1.2E+08 | 1.2E+08 | + | 2.03 | 1.02E−05 | 2.25 | 0.000144 | NM_012210 | 0.07 |
| chr9 | 1.26E+08 | 1.26E+08 | + | 0.19 | 0.465306 | 1.6 | 8.76E−05 | NM_026677 | 0.07 |
| chr13 | 33219125 | 33219385 | + | −0.01 | 0.470937 | 1.41 | 8.54E−05 | NM_015032 | 0.06 |
| chr19 | 55914629 | 55914903 | + | 1.96 | 0.009351 | 1.67 | 1.02E−05 | NM_014501 | 0.06 |
| chr2 | 54226039 | 54226282 | + | −1.03 | 0.107039 | 2.09 | 4.71E−07 | NM_014814 | 0.06 |
| chr21 | 35199006 | 35199187 | + | 0.57 | 0.230388 | 2.68 | 1.57E−05 | NM_001697 | 0.06 |
| chr6 | 34781221 | 34781593 | + | −0.77 | 0.163179 | 1.31 | 5.07E−05 | NM_017754 | 0.06 |
| chr7 | 44796472 | 44796875 | + | 0.52 | 0.164068 | 1.42 | 8.38E−06 | NM_174929 | 0.06 |
| chr17 | 47516437 | 47517063 | + | 1.95 | 3.75E−05 | 1.36 | 4.39E−10 | NM_002634 | 0.05 |
| chr17 | 47518750 | 47520003 | + | 2.86 | 7.79E−11 | 2.1 | 9.77E−25 | NM_002634 | 0.05 |
| chr17 | 47521248 | 47521451 | + | 0.46 | 0.334119 | 2.46 | 3.03E−06 | NM_002634 | 0.05 |
| chr17 | 47530054 | 47530228 | + | 1.92 | 0.003755 | 2.5 | 1.82E−06 | NM_002634 | 0.05 |
| chr2 | 39950736 | 39951042 | + | 0.74 | 0.041562 | 1.77 | 2.16E−07 | NR_028102 | 0.05 |
| chr2 | 2.17E+08 | 2.17E+08 | + | −0.46 | 0.175792 | 1.7 | 1.81E−06 | NM_000597 | 0.05 |
| chr20 | 48986691 | 48986940 | + | 1.26 | 0.011922 | 2.5 | 1.82E−06 | NR_034124 | 0.05 |
| chr4 | 1.84E+08 | 1.84E+08 | + | 2.04 | 0.099329 | 1.7 | 9.76E−05 | NM_001921 | 0.05 |
| chr5 | 1.54E+08 | 1.54E+08 | + | −0.54 | 0.203416 | 1.91 | 1.75E−05 | NM_015315 | 0.05 |
| chr9 | 1.24E+08 | 1.24E+08 | + | 0.11 | 0.482988 | 1.7 | 9.76E−05 | NM_016322 | 0.05 |
| chr19 | 18546658 | 18546851 | + | 1.41 | 0.004736 | 1.98 | 7.12E−06 | NM_001253389 | 0.04 |
| chr2 | 2.1E+08 | 2.1E+08 | + | 0.36 | 0.231846 | 1.85 | 7.28E−08 | NM_001039538 | 0.04 |
| chr5 | 1.53E+08 | 1.53E+08 | + | 0.84 | 0.148331 | 1.43 | 5.89E−05 | NM_000827 | 0.04 |
| chr7 | 1.51E+08 | 1.51E+08 | + | 1.42 | 3.40E−05 | 2.04 | 5.04E−22 | NM_005614 | 0.04 |
| chr9 | 2812940 | 2813110 | + | 0.87 | 0.110781 | 2.24 | 9.42E−06 | NM_014878 | 0.04 |
| chr1 | 1.04E+08 | 1.04E+08 | + | 1.48 | 6.30E−05 | 1.64 | 1.99E−14 | NM_080629 | 0.03 |
| chr1 | 1.04E+08 | 1.04E+08 | + | 2.09 | 2.83E−09 | 1.86 | 3.02E−12 | NM_080629 | 0.03 |
| chr1 | 1.68E+08 | 1.68E+08 | + | 0.57 | 0.230388 | 2.49 | 7.40E−09 | NM_001146191 | 0.03 |
| chr1 | 2.05E+08 | 2.05E+08 | + | 0.45 | 0.015307 | 2.52 | 7.56E−05 | NM_001242925 | 0.03 |
| chr17 | 61330184 | 61330440 | + | 1.54 | 0.022876 | 1.72 | 4.05E−05 | NM_001017917 | 0.03 |
| chr19 | 3609028 | 3609939 | + | 0.6 | 0.011601 | 1.61 | 1.33E−12 | NR_038865 | 0.03 |
| chr19 | 50531358 | 50531748 | + | 2.63 | 0.001965 | 1.85 | 2.21E−07 | NM_015428 | 0.03 |
| chr2 | 56114610 | 46115390 | + | 1.53 | 0.000621 | 1.36 | 5.99E−11 | NM_001039349 | 0.03 |
| chr2 | 56115899 | 56116414 | + | 1.59 | 0.000322 | 1.43 | 2.13E−07 | NM_001039349 | 0.03 |
| chr2 | 68474090 | 68474315 | + | 0.13 | 0.464479 | 2.46 | 3.03E−06 | NM_000945 | 0.03 |
| chr2 | 2.1E+08 | 2.5E+08 | + | 1.3 | 0.0345 | 4.27 | 6.94E−06 | NM_031847 | 0.03 |
| chr2 | 2.38E+08 | 2.38E+08 | + | 0.23 | 0.02581 | 3.34 | 2.36E−05 | NM_198189 | 0.03 |
| chr2 | 2.38E+08 | 2.38E+08 | + | 0.01 | 0.509 | 1.81 | 2.71E−05 | NM_198189 | 0.03 |
| chr22 | 30350374 | 30350528 | + | −0.54 | 0.261994 | 3.34 | 2.36E−05 | NM_153050 | 0.03 |
| chr3 | 1.44E+08 | 1.44E+08 | + | −0.3 | 0.29184 | 1.49 | 4.40E−05 | NM_001134470 | 0.03 |
| chr3 | 1.71E+08 | 1.71E+08 | + | 0.04 | 0.504309 | 1.96 | 6.59E−05 | NM_001161562 | 0.03 |
| chr3 | 1.71E+09 | 1.71E+08 | + | 0.78 | 0.044012 | 1.54 | 2.02E−05 | NM_001161562 | 0.03 |
| chr6 | 1.57E+08 | 1.57E+08 | + | −1.13 | 0.188905 | 1.66 | 6.47E−06 | NM_018452 | 0.03 |
| chr7 | 6290528 | 6290738 | + | 1.22 | 0.047091 | 2.03 | 1.19E−06 | NM_004227 | 0.03 |
| chr7 | 6308678 | 6308661 | + | 2.19 | 0.002873 | 1.95 | 2.71E−05 | NM_004227 | 0.03 |
| chr7 | 1.02E+08 | 1.02E+08 | + | −1.28 | 0.107269 | 1.79 | 7.19E−06 | NM_181552 | 0.03 |
| chr10 | 1.24E+08 | 1.24E+08 | + | 1.63 | 0.07633 | 2.42 | 5.02E−06 | NM_007041 | 0.02 |
| chr13 | 25706143 | 25706271 | + | 0 | 0.593079 | 3.46 | 7.48E−06 | NM_030979 | 0.02 |
| chr17 | 7729203 | 7729331 | + | 0 | 0.593079 | 4.27 | 6.94E−06 | NM_001080424 | 0.02 |
| chr19 | 47413985 | 47414334 | + | 1.33 | 0.082052 | 1.54 | 1.27E−05 | NM_004491 | 0.02 |
| chr6 | 68944702 | 68944830 | + | 1.46 | 0.222672 | 4.27 | 6.94E−06 | NM_001704 | 0.02 |
| chr7 | 73919392 | 73919699 | + | 1.11 | 0.036985 | 2.46 | 4.70E−07 | NM_005685 | 0.02 |
| chr7 | 1.41E+08 | 1.41E+08 | + | 1.92 | 0.035602 | 2.78 | 5.42E−06 | NM_018238 | 0.02 |
| chr9 | 1E+08 | 1E+08 | + | 0.54 | 0.111688 | 1.3 | 1.81E−05 | NM_002486 | 0.02 |
| chr1 | 43672570 | 43672957 | + | −0.03 | 0.460616 | 1.31 | 5.07E−05 | NM_001159936 | 0.01 |
| chr1 | 1.56E+08 | 1.56E+08 | + | 1.96 | 0.009351 | 2.2 | 5.95E−05 | NM_004632 | 0.01 |
| chr10 | 61316968 | 61317858 | + | 2.32 | 6.88E−09 | 2.06 | 4.02E−21 | NM_194298 | 0.01 |
| chr12 | 1.25E+08 | 1.25E+08 | + | −0.1 | 0.415961 | 3.34 | 2.30E−05 | NM_001082959 | 0.01 |
| chr12 | 1.25E+08 | 1.25E+08 | + | 1.78 | 0.149771 | 2.88 | 3.48E−05 | NM_001082959 | 0.01 |
| chr20 | 60622265 | 60622463 | + | −0.16 | 0.376068 | 1.52 | 7.61E−05 | NM_003185 | 0.01 |
| chr3 | 1.49E+08 | 1.49E+08 | + | 0.92 | 0.063595 | 1.88 | 6.65E−05 | NM_001188280 | 0.01 |
| chr4 | 3353361 | 3353573 | + | 1.36 | 0.044821 | 1.43 | 9.39E−05 | NM_188227 | 0.01 |
| chr5 | 1.42E+08 | 1.42E+08 | + | 0.61 | 0.00457 | 1.98 | 1.33E−10 | NM_001127496 | 0.01 |
| chr7 | 75963781 | 75063983 | + | 0.92 | 0.152933 | 2.01 | 4.17E−05 | NM_012479 | 0.01 |
| chr1 | 32580306 | 32580449 | + | −0.28 | 0.353275 | 3.05 | 6.68E−06 | NM_012316 | 0 |
| chr1 | 59275435 | 59275615 | + | 0.26 | 0.418658 | 2.41 | 3.22E−05 | NR_034015 | 0 |
| chr1 | 2.14E+08 | 2.14E+08 | + | 1.4 | 0.014239 | 1.71 | 1.79E−12 | NR_046189 | 0 |
| chr1 | 2.14E+08 | 2.14E+08 | + | 2.16 | 984E−09 | 1.5 | 3.44E−15 | NR_046189 | 0 |
| chr1 | 2.34E+08 | 2.34E+08 | + | 1.18 | 0.000393 | 1.43 | 8.94E−13 | NR_039625 | 0 |
| chr1 | 2.48E+08 | 2.48E+08 | + | 0.52 | 0.173082 | 1.67 | 2.51E−09 | NM_001004698 | 0 |
| chr10 | 10656127 | 10656264 | + | 1.46 | 0.222672 | 2.25 | 3.66E−05 | NR_027082 | 0 |
| chr10 | 24595354 | 24595567 | + | 1.46 | 0.109973 | 2.16 | 2.47E−05 | NR_030334 | 0 |
| chr10 | 31064331 | 31065108 | + | −0.36 | 0.136534 | 1.33 | 5.35E−09 | NM_183058 | 0 |
| chr10 | 45676458 | 45877069 | + | 2.23 | 1.18E−12 | 1.52 | 1.42E−08 | NR_036112 | 0 |
| chr10 | 50012899 | 50013139 | + | 0.46 | 0.404925 | 1.78 | 1.73E−05 | NM_001006939 | 0 |
| chr10 | 63554022 | 63554150 | + | 0.23 | 0.972582 | 4.58 | 3.23E−07 | NM_032199 | 0 |
| chr10 | 80636627 | 80636995 | + | 2.46 | 0.004621 | 1.49 | 1.735−05 | NR_015429 | 0 |
| chr10 | 1E+08 | 1E+08 | + | −0.54 | 0.261994 | 2.06 | 7.49E−07 | NR_031619 | 0 |
| chr10 | 1E+08 | 1E+08 | + | 2.93 | 1.73E−41 | 2.84 | 7.33E−54 | NR_031619 | 0 |
| chr10 | 1E+08 | 1E+08 | + | 4.1 | 6.48E−10 | 4.22 | 7.66E−19 | NR_031619 | 0 |
| chr10 | 1E+08 | 1E+08 | + | 1.84 | 2.03E−05 | 1.72 | 1.40E−13 | NR_031619 | 0 |
| chr10 | 1.26E+08 | 1.26E+08 | + | 0 | 0.593079 | 2.28 | 5.78E−08 | NM_001146340 | 0 |
| chr10 | 1.32E+08 | 1.32E+08 | + | 1.92 | 0.035602 | 2.583 | 1.73E−07 | NR_036179 | 0 |
| chr10 | 1.35E+08 | 1.35E+08 | + | −0.36 | 0.926622 | 2.15 | 9.64E−05 | NR_030170 | 0 |
| chr11 | 27641447 | 27641638 | + | 2.16 | 0.016043 | 2.38 | 8.30E−06 | NR_102708 | 0 |
| chr11 | 57792244 | 57792419 | + | 0.45 | 0.015307 | 4.78 | 2.72E−08 | NM_001005212 | 0 |
| chr11 | 57825789 | 57826451 | + | 0.4 | 0.14411 | 1.36 | 2.93E−09 | NM_001005186 | 0 |
| chr11 | 63645918 | 63646056 | + | 3.46 | 0.0011 | 3.34 | 2.36E−05 | NM_017490 | 0 |
| chr11 | 64173804 | 64173981 | + | −0.36 | 0.926622 | 3.25 | 7.10E−07 | NR_031602 | 0 |
| chr11 | 79115084 | 79115212 | + | −0.54 | 0.36033 | 2.93 | 2.01E−05 | NR_030598 | 0 |
| chr11 | 1.11E+08 | 1.11E+08 | + | 1.32 | 2.63E−05 | 1.43 | 1.35E−20 | NR_039712 | 0 |
| chr11 | 1.18E+08 | 1.18E+08 | + | −1.54 | 0.103108 | 2.93 | 2.01E−05 | NM_001558 | 0 |
| chr11 | 1.27E+08 | 1.27E+08 | + | −0.01 | 0.473517 | 1.78 | 3.84E−08 | NR_046986 | 0 |
| chr11 | 1.27E+08 | 1.27E+08 | + | −0.17 | 0.344028 | 1.38 | 1.08E−09 | NR_040078 | 0 |
| chr11 | 1.27E+08 | 1.27E+08 | + | 0.46 | 0.308555 | 1.76 | 1.10E−05 | NR_040078 | 0 |
| chr11 | 1.31E+08 | 1.31E+08 | + | 0.46 | 0.308555 | 1.66 | 1.73E−06 | NM_001271983 | 0 |
| chr11 | 1.35E+08 | 1.35E+08 | + | 1.1 | 0.001894 | 2.63 | 8.53E−16 | NR_033852 | 0 |
| chr11 | 1.35E+08 | 1.35E+08 | + | 0.41 | 0.197649 | 3.49 | 5.48E−31 | NR_033852 | 0 |
| chr11 | 1.35E+08 | 1.35E+08 | + | 2.3 | 2.10E−09 | 3.14 | 5.38E−29 | NR_033852 | 0 |
| chr11 | 1.35E+08 | 1.35E+08 | + | 2.1 | 6.05E−06 | 3.38 | 1.29E−07 | NR_033852 | 0 |
| chr11 | 1.35E+08 | 1.35E+08 | + | 1.46 | 0.032155 | 3.92 | 6.18E−15 | NR_033852 | 0 |
| chr11 | 1.35E+08 | 1.35E+08 | + | 3.13 | 1.79E−08 | 4.16 | 7.70E−43 | NR_033852 | 0 |
| chr11 | 1.35E+08 | 1.35E+08 | + | 3.36 | 2.77E−47 | 2.36 | 7.99E−54 | NR_033852 | 0 |
| chr11 | 1.35E+08 | 1.35E+08 | + | 3.94 | 1.53E−12 | 2.58 | 6.51E−07 | NR_033852 | 0 |
| chr11 | 1.35E+08 | 1.35E+08 | + | 1.19 | 0.113922 | 2.38 | 6.30E−11 | NR_033852 | 0 |
| chr12 | 11969210 | 11969339 | + | 0.09 | 0.461298 | 3.1 | 3.83E−06 | NR_046487 | 0 |
| chr12 | 13628016 | 13628202 | + | 0.04 | 0.513686 | 1.56 | 8.19E−05 | NR_036555 | 0 |
| chr12 | 13761072 | 13761200 | + | 3.46 | 0.0011 | 4.2 | 1.27E−05 | NR_036555 | 0 |
| chr12 | 14994804 | 14994960 | + | −0.13 | 0.422029 | 2.99 | 1.16E−05 | NM_021071 | 0 |
| chr12 | 25877258 | 25877388 | + | 0.78 | 0.201565 | 2.29 | 2.24E−05 | NM_001145727 | 0 |
| chr12 | 43681762 | 43682309 | + | 2.23 | 2.05E−05 | 2.78 | 8.39E−13 | NM_025003 | 0 |
| chr12 | 43683291 | 43683681 | + | 1.08 | 0.010278 | 2.89 | 3.40E−14 | NM_025003 | 0 |
| chr12 | 81601985 | 81602204 | + | 0.23 | 0.02581 | 2.11 | 3.97E−05 | NR_039848 | 0 |
| chr12 | 97651518 | 97651694 | + | −0.81 | 0.170714 | 3.68 | 7.23E−07 | NR_024037 | 0 |
| chr12 | 98479564 | 96480254 | + | 3.16 | 1.14E−08 | 2.35 | 2.44E−15 | NR_036189 | 0 |
| chr12 | 1.23E+08 | 1.23E+08 | + | 0.65 | 0.001184 | 1.35 | 2.80E−08 | NM_024667 | 0 |
| chr12 | 1.23E+08 | 1.23E+08 | + | 0.45 | 0.015307 | 2.92 | 1.07E−06 | NM_001002251 | 0 |
| chr13 | 26538091 | 26538326 | + | 0.82 | 0.10778 | 1.46 | 6.44E−05 | NM_001007538 | 0 |
| chr13 | 33396887 | 33397116 | + | 0.39 | 0.249424 | 1.6 | 8.76E−05 | NR_047020 | 0 |
| chr13 | 40519502 | 40519690 | + | 0.46 | 0.404925 | 2.2 | 5.95E−05 | NR_046870 | 0 |
| chr13 | 43198084 | 43198362 | + | 0.81 | 0.005384 | 2.1 | 4.28E−06 | NM_003701 | 0 |
| chr13 | 43325163 | 43325336 | + | 0.81 | 0.005384 | 2.41 | 3.22E−05 | NM_182508 | 0 |
| chr13 | 46470026 | 46470653 | + | 1.19 | 0.113922 | 1.51 | 3.26E−08 | NM_198849 | 0 |
| chr13 | 53739670 | 53739798 | + | 0.45 | 0.015307 | 4.88 | 7.84E−09 | NM_006418 | 0 |
| chr13 | 53743826 | 53744167 | + | 0.19 | 0.465306 | 2.95 | 6.17E−11 | NM_006418 | 0 |
| chr13 | 53744968 | 53745622 | + | 1.87 | 0.013694 | 3.23 | 1.70E−17 | NM_006418 | 0 |
| chr13 | 53771722 | 53773846 | + | 1 | 0.000149 | 1.45 | 4.70E−21 | NM_006418 | 0 |
| chr13 | 78949336 | 78949576 | + | 1.55 | 0.000234 | 1.65 | 9.30E−05 | NM_006237 | 0 |
| chr13 | 78977631 | 78977802 | + | 0.96 | 0.003193 | 4.73 | 5.05E−08 | NM_006237 | 0 |
| chr13 | 79361003 | 79361131 | + | −2.36 | 0.648257 | 3.34 | 2.36E−05 | NR_046869 | 0 |
| chr14 | 61933119 | 61933261 | + | −0.06 | 0.44608 | 2.78 | 5.42E−06 | NR_039985 | 0 |
| chr14 | 85426819 | 85426979 | + | 0.45 | 0.015307 | 2.93 | 2.01E−05 | NR_102737 | 0 |
| chr14 | 1.05E+08 | 1.05E+08 | + | −1.13 | 0.025762 | 1.52 | 1.27E−06 | NM_001008404 | 0 |
| chr15 | 39218732 | 39218942 | + | 0.04 | 0.529972 | 2.64 | 2.45E−06 | NM_207444 | 0 |
| chr15 | 60159346 | 60159882 | + | 0.19 | 0.424653 | 1.75 | 3.76E−08 | NM_012182 | 0 |
| chr15 | 62757376 | 62757514 | + | −1.13 | 0.108085 | 2.92 | 1.07E−06 | NR_026897 | 0 |
| chr15 | 74574650 | 74674806 | + | 1.33 | 0.082052 | 2.73 | 9.25E−06 | NM_025055 | 0 |
| chr15 | 75982744 | 75982924 | + | 1.04 | 0.04895 | 3.1 | 3.83E−06 | NM_001897 | 0 |
| chr15 | 89154155 | 89154622 | + | 0.46 | 0.365076 | 2.08 | 5.91E−09 | NR_029606 | 0 |
| chr16 | 55779899 | 55780066 | + | −0.37 | 0.283497 | 2.07 | 6.37E−05 | NR_003276 | 0 |
| chr16 | 55823601 | 55824462 | + | 0.86 | 0.027931 | 1.74 | 1.04E−11 | NR_003276 | 0 |
| chr16 | 57841079 | 57841208 | + | 1.46 | 0.222672 | 2.93 | 2.01E−05 | NM_001278081 | 0 |
| chr16 | 57972671 | 57972899 | + | 2.26 | 0.065139 | 1.68 | 1.62E−05 | NM_001297 | 0 |
| chr16 | 73581552 | 73582284 | + | 1.64 | 0.000139 | 2.05 | 1.60E−12 | NR_038234 | 0 |
| chr16 | 73705089 | 73705396 | + | 0.68 | 0.280156 | 1.73 | 1.68E−05 | NR_038234 | 0 |
| chr16 | 85387355 | 85387561 | + | −0.35 | 0.30382 | 2.58 | 4.50E−05 | NR_049816 | 0 |
| chr17 | 18973077 | 18973205 | + | −0.96 | 0.104912 | 3.73 | 4.01E−07 | NM_006613 | 0 |
| chr17 | 30023224 | 30023358 | + | 0.04 | 0.529972 | 2.81 | 5.99E−05 | NR_029856 | 0 |
| chr17 | 68106484 | 68106709 | + | 1.16 | 0.08465 | 1.59 | 5.50E−05 | NM_018658 | 0 |
| chr17 | 71851797 | 71852183 | + | 0.68 | 0.126482 | 1.52 | 1.27E−06 | NR_027146 | 0 |
| chr17 | 75880302 | 75880439 | + | −0.54 | 0.3033 | 2.93 | 2.01E−05 | NR_028337 | 0 |
| chr17 | 77644303 | 77644849 | + | 1.5 | 0.002493 | 2.68 | 1.12E−13 | NR_039893 | 0 |
| chr18 | 6541217 | 6541352 | + | 0.78 | 0.301098 | 2 | 1.88E−06 | NM_001243517 | 0 |
| chr18 | 14912993 | 14913127 | + | −0.36 | 0.926622 | 3.38 | 1.29E−07 | NR_036153 | 0 |
| chr18 | 14971594 | 14971796 | + | 1.34 | 0.000666 | 2.38 | 5.51E−08 | NR_036153 | 0 |
| chr18 | 14972405 | 14972547 | + | 1.36 | 0.044821 | 2.68 | 1.57E−05 | NR_036153 | 0 |
| chr18 | 14986724 | 14986915 | + | 3.46 | 0.0011 | 2.96 | 6.20E−07 | NR_036153 | 0 |
| chr18 | 15067063 | 15067959 | + | 1.96 | 4.26E−07 | 1.56 | 4.63E−12 | NR_036153 | 0 |
| chr18 | 15070052 | 15070235 | + | 0.64 | 0.009079 | 2.73 | 9.25E−06 | NR_036153 | 0 |
| chr18 | 27197929 | 27198168 | + | 1.92 | 0.035602 | 1.49 | 7.02E−05 | NR_031684 | 0 |
| chr18 | 27292843 | 27292992 | + | 0.9 | 0.083608 | 1.88 | 2.74E−05 | NR_031684 | 0 |
| chr18 | 43414021 | 43414945 | + | −0.17 | 0.314291 | 1.41 | 1.25E−17 | NM_213602 | 0 |
| chr18 | 56155573 | 56155747 | + | −0.16 | 0.376068 | 1.8 | 6.58E−05 | NR_039899 | 0 |
| chr18 | 69776124 | 69776263 | + | 1.57 | 0.041179 | 3.73 | 4.01E−07 | NM_182511 | 0 |
| chr19 | 18401815 | 18401953 | + | −0.18 | 0.381896 | 2.46 | 1.94E−05 | NR_036155 | 0 |
| chr2 | 17454049 | 17454858 | + | 1.17 | 0.027729 | 3.59 | 1.76E−21 | NM_001099218 | 0 |
| chr2 | 21473721 | 21474996 | + | 0.65 | 0.083141 | 2.49 | 8.28E−26 | NM_000384 | 0 |
| chr2 | 21479032 | 21479219 | + | 1.26 | 0.156387 | 3.29 | 4.03E−07 | NM_000384 | 0 |
| chr2 | 21482960 | 21483383 | + | 2.26 | 0.010657 | 4.12 | 1.66E−13 | NM_000384 | 0 |
| chr2 | 21484000 | 21485005 | + | 4.57 | 5.78E−14 | 3.96 | 7.17E−33 | NM_000384 | 0 |
| chr2 | 21485602 | 21486129 | + | 2.83 | 2.77E−05 | 3.06 | 1.47E−16 | NM_000384 | 0 |
| chr2 | 21512539 | 21512779 | + | 0.45 | 0.015307 | 5.01 | 1.21E−09 | NM_000384 | 0 |
| chr2 | 21812857 | 21813002 | + | 0.78 | 0.301098 | 2.19 | 3.14E−07 | NR_038837 | 0 |
| chr2 | 33090422 | 33090693 | + | 0.84 | 0.148331 | 1.82 | 4.67E−06 | NR_027099 | 0 |
| chr2 | 56355601 | 56355735 | + | 0.96 | 0.003193 | 2.32 | 8.53E−08 | NM_001080433 | 0 |
| chr2 | 1.35E+08 | 1.35E+08 | + | 0.81 | 0.93262 | 2.52 | 7.56E−05 | NR_037450 | 0 |
| chr2 | 1.83E+08 | 1.83E+08 | + | 1.68 | 0.028759 | 2.05 | 2.62E−05 | NM_001080545 | 0 |
| chr2 | 1.86E+08 | 1.86E+08 | + | 1.34 | 0.000666 | 2.32 | 3.54E−06 | NM_173651 | 0 |
| chr2 | 2.15E+08 | 2.15E+08 | + | 1.04 | 0.325105 | 3.96 | 2.06E−08 | MR_047698 | 0 |
| chr2 | 2.4E+08 | 2.4E+08 | + | 0.04 | 0.492338 | 2.28 | 5.78E−06 | NR_036229 | 0 |
| chr20 | 49971625 | 49972134 | + | 0.9 | 0.083608 | 1.35 | 3.58E−06 | NR_036182 | 0 |
| chr20 | 56534294 | 56534456 | + | 0.68 | 0.182687 | 2.01 | 4.17E−05 | NR_039757 | 0 |
| chr21 | 41425428 | 41425556 | + | −0.36 | 0.926622 | 3.2 | 7.38E−05 | NR_039917 | 0 |
| chr21 | 41429262 | 41429575 | + | 2.36 | 0.007037 | 2.06 | 8.33E−08 | NR_039917 | 0 |
| chr21 | 46011639 | 46011902 | + | 0.46 | 0.463277 | 1.6 | 8.76E−05 | NM_198688 | 0 |
| chr22 | 48711353 | 48712110 | + | 2.78 | 4.29E−05 | 1.81 | 9.31E−11 | NR_036172 | 0 |
| chr22 | 48932686 | 48933292 | + | 0.89 | 0.048052 | 1.78 | 9.11E−08 | NR_038917 | 0 |
| chr3 | 13728707 | 13729763 | + | 1.18 | 0.001315 | 1.84 | 3.04E−17 | NR_027103 | 0 |
| chr3 | 13731953 | 13732599 | + | 0.87 | 0.062557 | 1.68 | 1.07E−09 | NR_027103 | 0 |
| chr3 | 59231154 | 59231995 | + | 2.04 | 0.006345 | 1.99 | 1.02E−14 | NM_198463 | 0 |
| chr3 | 64875851 | 64876079 | + | 0.19 | 0.424653 | 1.75 | 2.65E−05 | NR_030317 | 0 |
| chr3 | 86562326 | 86562565 | + | 1.63 | 0.07633 | 1.62 | 3.67E−05 | NR_046939 | 0 |
| chr3 | 86703026 | 86703274 | + | 1.63 | 0.07633 | 1.67 | 3.87E−05 | NR_046939 | 0 |
| chr3 | 1.01E+08 | 1.01E+08 | + | 0.59 | 0.149458 | 2.36 | 5.33E−05 | NM_014415 | 0 |
| chr3 | 1.12E+08 | 1.12E+08 | + | −1.03 | 0.107039 | 1.68 | 6.15E−05 | NM_183061 | 0 |
| chr3 | 1.34E+08 | 1.34E+08 | + | 2.26 | 0.065139 | 2.68 | 1.57E−05 | NM_178554 | 0 |
| chr3 | 1.53E+08 | 1.53E+08 | + | −0.54 | 0.281197 | 3.46 | 7.46E−06 | NM_001101337 | 0 |
| chr3 | 1.97E+08 | 1.97E+08 | + | −1.13 | 0.040692 | 1.72 | 4.05E−05 | NR_039960 | 0 |
| chr4 | 46707646 | 46707957 | + | 2.36 | 0.007037 | 1.54 | 1.27E−05 | NM_130902 | 0 |
| chr4 | 90630842 | 90631320 | + | 0.92 | 0.063595 | 1.41 | 7.38E−09 | NR_045481 | 0 |
| chr4 | 1.26E+08 | 1.26E+08 | + | 0.04 | 0.513686 | 3.2 | 1.25E−06 | NR_031746 | 0 |
| chr4 | 1.56E+08 | 1.56E+08 | + | 1.04 | 0.156144 | 2.63 | 2.67E−05 | NM_000910 | 0 |
| chr4 | 1.56E+08 | 1.56E+08 | + | 1.04 | 0.218994 | 2.88 | 1.00E−07 | NM_000910 | 0 |
| chr4 | 1.58E+08 | 1.58E+08 | + | 0.13 | 0.464479 | 2.36 | 5.33E−05 | NR_026992 | 0 |
| chr4 | 1.89E+08 | 1.89E+08 | + | 0.58 | 0.088758 | 1.36 | 4.60E−09 | NM_174900 | 0 |
| chr5 | 9165629 | 9165809 | + | 0.81 | 0.93262 | 2.93 | 2.01E−05 | NR_039779 | 0 |
| chr5 | 9166378 | 9167506 | + | 2.04 | 1.08E−06 | 1.56 | 1.19E−13 | NR_039779 | 0 |
| chr5 | 9623426 | 9623573 | + | −0.54 | 0.3033 | 2.68 | 1.57E−05 | NM_019599 | 0 |
| chr5 | 16887753 | 16887906 | + | −0.22 | 0.38435 | 2.58 | 6.51E−07 | NM_012334 | 0 |
| chr5 | 86415644 | 86416216 | + | 1.87 | 1.62E−13 | 2.14 | 3.90E−10 | NR_036243 | 0 |
| chr5 | 86421717 | 86421976 | + | 1.97 | 9.83E−07 | 3.65 | 2.246−09 | NR_036243 | 0 |
| chr5 | 86433127 | 86434310 | + | 2.84 | 8.85E−20 | 1.31 | 1.95E−10 | NR_036243 | 0 |
| chr5 | 86484078 | 86484518 | + | 2.21 | 0.00054 | 1.43 | 2.13E−07 | NR_036249 | 0 |
| chr5 | 1.46E+08 | 1.46E+08 | + | 3.16 | 0.004461 | 1.96 | 1.02E−08 | NM_194251 | 0 |
| chr5 | 1.46E+08 | 1.46E+08 | + | 0.13 | 0.429293 | 1.6 | 8.76E−05 | NR_047115 | 0 |
| chr5 | 1.52E+08 | 1.52E+08 | + | 2.46 | 0.042327 | 3.68 | 1.25E−09 | NM_020167 | 0 |
| chr5 | 1.52E+08 | 1.52E+08 | + | 2.87 | 1.79E−05 | 1.61 | 1.25E−08 | NM_020167 | 0 |
| chr5 | 1.54E+08 | 1.54E+08 | + | 1.46 | 0.005935 | 1.77 | 4.59E−08 | NR_037897 | 0 |
| chr5 | 1.6E+08 | 1.6E+08 | + | 1.92 | 0.003755 | 1.38 | 6.92E−06 | NR_036095 | 0 |
| chr5 | 1.7E+08 | 1.7E+08 | + | 0.04 | 0.504309 | 1.71 | 2.02E−10 | NM_004137 | 0 |
| chr6 | 22789703 | 22789842 | + | −1.18 | 0.025593 | 2.52 | 7.56E−05 | NM_138574 | 0 |
| chr6 | 44499354 | 44499687 | + | 1.78 | 0.01992 | 1.51 | 1.88E−05 | NR_039785 | 0 |
| chr6 | 1.32E+08 | 1.32E+08 | + | 1.73 | 0.001876 | 2.14 | 2.24E−25 | NR_038981 | 0 |
| chr7 | 19564732 | 19564940 | + | −0.59 | 0.05591 | 2.23 | 2.44E−06 | NR_036101 | 0 |
| chr7 | 20073788 | 20073917 | + | −0.51 | 0.00454 | 3.5 | 2.29E−08 | NR_046756 | 0 |
| chr7 | 43178657 | 43179090 | + | 1.33 | 0.082052 | 2.65 | 3.11E−14 | NR_037508 | 0 |
| chr7 | 1.28E+08 | 1.28E+08 | + | −0.54 | 0.281197 | 2.31 | 8.78E−05 | NR_027330 | 0 |
| chr7 | 1.38E+08 | 1.38E+08 | + | 0.46 | 0.267559 | 1.55 | 2.18E−06 | NM_004717 | 0 |
| chr7 | 1.43E+08 | 1.43E+08 | + | 1.81 | 4.89E−05 | 1.78 | 1.92E−06 | NM_001001667 | 0 |
| chr7 | 1.56E+08 | 1.56E+08 | + | 0.86 | 0.016783 | 2.37 | 2.37E−09 | NM_000193 | 0 |
| chr7 | 1.57E+08 | 1.57E+08 | + | 1.26 | 0.156387 | 2.52 | 7.56E−05 | NR_029689 | 0 |
| chr7 | 1.57E+08 | 1.57E+08 | + | 1.64 | 1.70E+06 | 2.56 | 1.81E−17 | NR_029689 | 0 |
| chr7 | 1.57E+08 | 1.57E+08 | + | 0.26 | 0.222416 | 1.83 | 2.91E−09 | NR_029689 | 0 |
| chr7 | 1.57E+08 | 1.57E+08 | + | −0.13 | 0.422029 | 3.1 | 3.83E−06 | NR_029689 | 0 |
| chr8 | 23552943 | 23553115 | + | −0.54 | 0.329466 | 2.12 | 1.03E−05 | NM_001136271 | 0 |
| chr8 | 24591737 | 24592027 | + | 0.78 | 0.104428 | 2.0 | 5.55E−08 | NM_005382 | 0 |
| chr8 | 24768615 | 24769206 | + | 0.51 | 0.064611 | 1.66 | 1.80E−12 | NM_005382 | 0 |
| chr8 | 39439535 | 39439711 | + | 1.04 | 0.156144 | 2.31 | 8.78E−05 | NM_001190956 | 0 |
| chr8 | 69547154 | 69547337 | + | 2.46 | 0.004821 | 3.05 | 6.54E−09 | NR_038877 | 0 |
| chr8 | 69552599 | 69553402 | + | 1.45 | 0.000395 | 2.82 | 3.25E−20 | NR_038877 | 0 |
| chr8 | 69610620 | 69611398 | + | 3.79 | 2.08E−27 | 3.45 | 8.54E−28 | NR_038877 | 0 |
| chr8 | 69612680 | 69612862 | + | 2.63 | 0.027294 | 4.01 | 1.13E−08 | NR_038877 | 0 |
| chr8 | 69670588 | 69870752 | + | 2.03 | 1.02E−05 | 3.2 | 1.25E−06 | NR_039986 | 0 |
| chr8 | 69671810 | 69672516 | + | 4.28 | 3.02E−21 | 2.73 | 1.31E−18 | NR_039986 | 0 |
| chr8 | 69698803 | 69699391 | + | 2.01 | 1.78E−05 | 1.89 | 5.40E−13 | NR_039983 | 0 |
| chr8 | 69765707 | 69766261 | + | 1.52 | 0.007411 | 2.7 | 6.43E−16 | NR_039986 | 0 |
| chr8 | 69828895 | 69829029 | + | 2.63 | 0.027294 | 2.54 | 6.31E−09 | NR_039986 | 0 |
| chr8 | 69942535 | 69943233 | + | 2.23 | 2.05E−05 | 1.72 | 3.14E−10 | NR_039986 | 0 |
| chr8 | 69946021 | 69946257 | + | 2.63 | 0.027294 | 3.68 | 1.25E−09 | NR_039986 | 0 |
| chr8 | 70178481 | 70178636 | + | 0.81 | 0.005384 | 3.63 | 1.30E−06 | NR_039986 | 0 |
| chr8 | 70189511 | 70190321 | + | 0.78 | 0.058164 | 3.22 | 6.38E−21 | NR_039986 | 0 |
| chr8 | 1.11E+08 | 1.11E+08 | + | 0.97 | 0.002477 | 1.38 | 5.32E−10 | NM_014379 | 0 |
| chr8 | 1.25E+08 | 1.25E+08 | + | 0.45 | 0.015307 | 2.55 | 1.66E−08 | NM_001039112 | 0 |
| chr8 | 1.29E+08 | 1.29E+08 | + | 1.14 | 0.016598 | 1.82 | 1.64E−08 | NR_031610 | 0 |
| chr8 | 1.43E+08 | 1.43E+08 | + | 1.78 | 1.12E−07 | 2.55 | 3.13E−30 | NM_207414 | 0 |
| chr8 | 1.43E+08 | 1.43E+08 | + | 1.12 | 0.001111 | 2.4 | 3.16E−26 | NM_207414 | 0 |
| chr8 | 1.43E+08 | 1.43E+08 | + | 1.87 | 2.79E−143 | 1.63 | 9.63E−180 | NM_207414 | 0 |
| chr8 | 1.425+08 | 1.43E+08 | + | 1.46 | 0.010305 | 3.83 | 1.23E−07 | NM_207414 | 0 |
| chr8 | 1.43E+08 | 1.43E+08 | + | 2.08 | 1.20E−63 | 2.55 | 9.09E−130 | NM_207414 | 0 |
| chr8 | 1.43E+08 | 1.43E+08 | + | 1.7 | 0.01132 | 2.88 | 1.84E−06 | NM_207414 | 0 |
| chr8 | 1.43E+08 | 1.43E+08 | + | 0.87 | 0.210905 | 3.58 | 7.18E−09 | NM_207414 | 0 |
| chr8 | 1.43E+08 | 1.43E+08 | + | 1.33 | 0.019547 | 2.96 | 1.51E−20 | NM_207414 | 0 |
| chr8 | 1.43E+08 | 1.43E+08 | + | 0.86 | 0.010197 | 1.53 | 1.13E−10 | NM_207414 | 0 |
| chr8 | 1.43E+08 | 1.43E+08 | + | 1.48 | 2.01E−24 | 1.66 | 1.10E−49 | NM_207414 | 0 |
| chr8 | 1.43E+08 | 1.43E+08 | + | 0.74 | 0.000836 | 1.99 | 4.61E−28 | NM_207414 | 0 |
| chr8 | 1.43E+08 | 1.43E+08 | + | 1.01 | 0.005043 | 1.83 | 2.21E−10 | NM_207414 | 0 |
| chr8 | 1.43E+08 | 1.43E+08 | + | 0.92 | 0.013438 | 2.68 | 1.20E−10 | NM_207414 | 0 |
| chr8 | 1.43E+08 | 1.43E+08 | + | 1.95 | 3.98E−11 | 2.85 | 1.16E−61 | NM_207414 | 0 |
| chr9 | 16515973 | 16518371 | + | 1.04 | 0.114476 | 1.35 | 3.96E−05 | NR_073471 | 0 |
| chr9 | 16522104 | 16522831 | + | 2.08 | 9.74E−05 | 1.67 | 1.44E−08 | NR_073471 | 0 |
| chr9 | 73363819 | 73364036 | + | 0.94 | 0.113163 | 2.03 | 1.09E−05 | NR_029621 | 0 |
| chr9 | 78104259 | 78104394 | + | 1.78 | 0.149771 | 2.31 | 8.78E−05 | NR_031679 | 0 |
| chr9 | 92238839 | 92238986 | + | 0.45 | 0.015307 | 3.29 | 4.03E−07 | NR_024280 | 0 |
| chr9 | 92457498 | 92458145 | + | 0.98 | 0.049001 | 1.98 | 1.59E−14 | NR_924280 | 0 |
| chr9 | 92504302 | 92504458 | + | 0.87 | 0.210905 | 2.5 | 1.82E−06 | NR_024280 | 0 |
| chr9 | 92691840 | 92692890 | + | 1.3 | 0.004056 | 1.62 | 4.49E−11 | NR_036253 | 0 |
| chr9 | 92764997 | 92765272 | + | 1.33 | 0.082052 | 1.52 | 7.61E−05 | NR_036253 | 0 |
| chr9 | 1.2E+08 | 1.2E+08 | + | −0.36 | 0.926622 | 3.58 | 2.33E−06 | NR_003708 | 0 |
| chr9 | 1.2E+08 | 1.2E+08 | + | 2.04 | 0.02399 | 2.72 | 4.00E−13 | NR_003708 | 0 |
| chr9 | 1.2E+08 | 1.2E+08 | + | 0.46 | 0.404925 | 1.99 | 3.61E−11 | NR_003708 | 0 |
| chr9 | 1.37E+08 | 1.37E+08 | + | 0.4 | 0.234774 | 1.72 | 9.93E−09 | NR_039816 | 0 |
| chr9 | 1.37E+08 | 1.37E+08 | + | 1.2 | 0.002108 | 1.3 | 2.87E−05 | NR_039816 | 0 |
| chrX | 1.3E+08 | 1.3E+08 | + | 4.29 | 2.13E−11 | 2.04 | 1.68E−09 | NM_178471 | 0 |
| chrX | 1.32E+08 | 1.32E+08 | + | −2.36 | 0.648257 | 3.4 | 1.33E−05 | NR_046891 | 0 |
| chrX | 1.45E+08 | 1.45E+08 | + | 1.78 | 0.052385 | 2.2 | 1.53E−05 | NR_030581 | 0 |
| chrY | 6003942 | 6004091 | + | 1.73 | 8.24E−05 | 4.27 | 6.94E−06 | NR_003593 | 0 |
| chr14 | 28812828 | 28812959 | + | 0.11 | 0.462988 | 2.35 | 9.04E−08 | NM_005249 | −0.01 |
| chr11 | 12757237 | 12757492 | + | −0.96 | 0.063073 | 1.91 | 7.26E−06 | NM_021961 | −0.02 |
| chr6 | 680566 | 681209 | + | −0.1 | 0.404446 | 1.3 | 1.97E−07 | NM_018303 | −0.02 |
| chr6 | 1.48E+08 | 1.48E+08 | + | 0.46 | 0.404925 | 2.19 | 3.14E−07 | NM_001030060 | −0.02 |
| chr7 | 55843574 | 55844292 | + | 0.96 | 1.36E−27 | 1.56 | 3.47E−46 | NR_003949 | −0.02 |
| chr1 | 1.81E+08 | 1.81E+08 | + | 0.73 | 0.017699 | 1.34 | 3.23E−06 | NM_020950 | −0.03 |
| chr12 | 95804840 | 95805100 | + | −0.32 | 0.326824 | 2.3 | 9.22E−07 | NM_006838 | −0.03 |
| chr19 | 14669628 | 14669765 | + | 0.36 | 0.303275 | 3.68 | 7.23E−07 | NM_004146 | −0.03 |
| chr22 | 22177308 | 22177515 | + | −1.54 | 0.060085 | 2.41 | 3.22E−05 | NM_002745 | −0.03 |
| chr3 | 1.54E+08 | 1.54E+08 | + | 1.34 | 0.000831 | 1.3 | 8.05E−05 | NM_001114397 | −0.03 |
| chr3 | 1.54E+08 | 1.54E+08 | + | −0.22 | 0.35557 | 2.58 | 4.50E−05 | NM_001114397 | −0.03 |
| chr5 | 86621733 | 86621861 | + | 1.04 | 0.325105 | 1.98 | 2.21E−06 | NM_022650 | −0.03 |
| chr1 | 68674563 | 66674892 | + | 1.04 | 0.156144 | 1.46 | 6.44E−05 | NM_001193334 | −0.04 |
| chr12 | 19609490 | 19609927 | + | 1.04 | 0.06437 | 1.37 | 5.70E−07 | NM_001267043 | −0.04 |
| chr14 | 78255769 | 78255979 | + | 1.46 | 0.058426 | 1.78 | 1.73E−05 | NM_020421 | −0.04 |
| chr19 | 39862553 | 39863647 | + | 1.77 | 1.33E−20 | 1.6 | 2.23E−16 | NM_001256826 | −0.04 |
| chr19 | 56592633 | 56592902 | + | −0.32 | 0.326824 | 1.78 | 1.92E−06 | NM_001002836 | −0.04 |
| chr20 | 11776581 | 11776709 | + | 0.46 | 0.463277 | 3.52 | 4.18E−06 | NM_181443 | −0.04 |
| chr20 | 11888084 | 11888218 | + | −0.03 | 0.460616 | 2.01 | 4.17E−05 | NM_014962 | −0.04 |
| chr20 | 12094211 | 12094431 | + | 1.04 | 0.114478 | 1.72 | 4.05E−05 | NM_014962 | −0.04 |
| chr4 | 1.52E+08 | 1.52E+08 | + | −1.51 | 1.18E−05 | 1.71 | 0.001489 | NM_006439 | −0.04 |
| chrX | 1.28E+08 | 1.28E+08 | + | 0.87 | 0.110781 | 1.35 | 1.30E−06 | NM_003069 | −0.04 |
| chr1 | 2.01E+08 | 2.01E+08 | + | 1.46 | 0.109973 | 3.88 | 6.78E−08 | NM_203459 | −0.05 |
| chr12 | 50609599 | 50609727 | + | 1.57 | 0.041179 | 2.81 | 5.99E−05 | NM_001113547 | −0.05 |
| chr15 | 83480940 | 83481157 | + | 2.04 | 0.02399 | 1.99 | 1.72E−05 | NM_001080435 | −0.05 |
| chr19 | 18458753 | 18458995 | + | 0.46 | 0.143077 | 1.9 | 5.25E−07 | NM_017712 | −0.05 |
| chr2 | 1.47E+08 | 1.47E+08 | + | 1.16 | 0.08465 | 2.58 | 9.92E−09 | NR_026904 | −0.05 |
| chr20 | 25201278 | 25201452 | + | −0.13 | 0.39179 | 2.23 | 2.44E−06 | NM_001114089 | −0.05 |
| chr22 | 26964283 | 26964493 | + | 0.46 | 0.334119 | 1.8 | 6.58E−05 | NM_001008566 | −0.05 |
| chr7 | 2847783 | 2848050 | + | 0.06 | 0.121103 | 1.7 | 5.53E−08 | NM_007353 | −0.05 |
| chr1 | 2.36E+08 | 2.36E+08 | + | 0.26 | 0.362534 | 4.52 | 5.98E−07 | NM_002508 | −0.06 |
| chr10 | 13987015 | 13987199 | + | −1.54 | 0.103108 | 2.15 | 9.64E−05 | NM_003675 | −0.06 |
| chr12 | 32362947 | 32363194 | + | 0.46 | 0.208746 | 1.84 | 1.76E−05 | NM_001714 | −0.06 |
| chr12 | 75940067 | 75940196 | + | −3.13 | 0.009517 | 3.05 | 6.68E−06 | NM_007043 | −0.06 |
| chr14 | 22723176 | 22723469 | + | 1.86 | 0.000267 | 1.5 | 1.86E−06 | NM_001344 | −0.06 |
| chr14 | 22838657 | 22838906 | + | 1.57 | 0.041179 | 1.51 | 1.88E−05 | NM_001344 | −0.06 |
| chr19 | 11012107 | 11012498 | + | 1.48 | 6.30E−05 | 1.31 | 2.62E−06 | NM_024029 | −0.06 |
| chr19 | 15386558 | 15386748 | + | 0.87 | 0.062557 | 1.58 | 2.17E−05 | NM_014299 | −0.06 |
| chr5 | 1.51E+08 | 1.51E+08 | + | 0.15 | 0.403369 | 3.83 | 1.23E−07 | NM_003118 | −0.06 |
| chr5 | 1.71E+08 | 1.71E+08 | + | 1.26 | 0.061907 | 2.32 | 1.48E−07 | NM_012300 | −0.06 |
| chr9 | 1.25E+08 | 1.25E+08 | + | 2.63 | 0.027294 | 1.72 | 4.05E−05 | NM_014222 | −0.06 |
| chr19 | 18262164 | 18262350 | + | −0.04 | 0.452279 | 2.03 | 1.09E−05 | NM_005027 | −0.07 |
| chr2 | 85985751 | 85986035 | + | 2.63 | 0.027294 | 2.09 | 4.71E−07 | NM_032827 | −0.07 |
| chr2 | 85989124 | 85989252 | + | −1.22 | 0.064934 | 4.52 | 5.98E−07 | NM_032827 | −0.07 |
| chr2 | 85994046 | 85994332 | + | −0.37 | 0.283497 | 1.67 | 7.30E−07 | NM_032827 | −0.07 |
| chr6 | 1.06E+08 | 1.06E+08 | + | 1.73 | 8.24E−05 | 1.35 | 0.000178 | NM_002726 | −0.07 |
| chr6 | 1.06E+08 | 1.06E+08 | + | −1.54 | 0.187264 | 2.58 | 4.50E−05 | NM_002726 | −0.07 |
| chr15 | 40126126 | 40126256 | + | 0.04 | 0.529972 | 2.51 | 1.16E−05 | NM_001271855 | −0.08 |
| chr5 | 31413148 | 31413415 | + | 0.66 | 0.121103 | 2.62 | 3.68E−08 | NM_001100412 | −0.08 |
| chrX | 1.33E+08 | 1.33E+08 | + | 2.93 | 4.28E−30 | 2.38 | 1.90E−19 | NR_029523 | −0.08 |
| chrX | 1.33E+08 | 1.33E+08 | + | 2.82 | 1.27E−20 | 2.27 | 4.50E−14 | NR_029523 | −0.08 |
| chr16 | 28774465 | 28774795 | + | 0.23 | 0.391248 | 2.06 | 7.49E−07 | NM_001099651 | −0.09 |
| chr1 | 1.51E+08 | 1.51E+08 | + | 1.59 | 0.009203 | 1.63 | 5.84E−05 | NM_144618 | −0.1 |
| chr16 | 580679 | 580827 | + | −1.28 | 0.107269 | 2.78 | 5.42E−06 | NM_005632 | −0.1 |
| chr18 | 4549715 | 4550035 | + | −0.13 | 0.411748 | 2.57 | 2.54E−11 | NM_004746 | −0.1 |
| chr22 | 32356606 | 32356897 | + | 0.87 | 0.210905 | 1.76 | 1.10E−05 | NM_015372 | −0.1 |
| chr4 | 1.57E+08 | 1.57E+08 | + | 0.18 | 0.316858 | 1.81 | 1.12E−05 | NM_016205 | −0.1 |
| chr4 | 1.58E+08 | 1.58E+08 | + | 0.36 | 0.303275 | 2.11 | 3.97E−05 | NM_016205 | −0.1 |
| chr4 | 1.58E+08 | 1.58E+08 | + | −0.16 | 0.369491 | 3.23 | 1.26E−08 | NM_016205 | −0.1 |
| chr8 | 1.22E+08 | 1.22E+08 | + | 0.75 | 0.075101 | 2.11 | 3.97E−05 | NM_002514 | −0.1 |
| chr10 | 45546217 | 45546405 | + | 1.04 | 0.156144 | 2.29 | 2.42E−07 | NM_001039380 | −0.11 |
| chr10 | 86097700 | 86097828 | + | 0.46 | 0.334119 | 2.88 | 3.48E−05 | NM_018999 | −0.11 |
| chr17 | 79972458 | 79972586 | + | −0.18 | 0.381896 | 2.2 | 5.95E−05 | NM_144998 | −0.11 |
| chr19 | 15518848 | 15518996 | + | 2.83 | 2.77E−05 | 1.66 | 0.000575 | NM_014371 | −0.11 |
| chr7 | 69271595 | 69271909 | + | −0.67 | 0.128489 | 1.65 | 9.30E−05 | NM_001127232 | −0.11 |
| chr18 | 55836086 | 55836255 | + | 1.7 | 0.01132 | 2.88 | 1.84E−06 | NM_001144968 | −0.12 |
| chr20 | 45542430 | 45542609 | + | 0.56 | 0.128974 | 2.05 | 2.62E−05 | NM_172110 | −0.12 |
| chr20 | 45616006 | 45616165 | + | −0.54 | 0.215984 | 1.65 | 9.30E−05 | NM_172110 | −0.12 |
| chr1 | 88666310 | 88666438 | + | 1.46 | 0.222672 | 2.25 | 3.66E−05 | NM_006256 | −0.13 |
| chr15 | 29363501 | 29363677 | + | 1.81 | 0.834416 | 2.81 | 5.99E−05 | NM_005503 | −0.13 |
| chr20 | 17630485 | 17630744 | + | −0.39 | 0.265331 | 1.52 | 2.99E−05 | NM_001042576 | −0.13 |
| chr3 | 1.97E+08 | 1.97E+08 | + | 0.54 | 0.106535 | 1.48 | 4.43E−08 | NM_004051 | −0.13 |
| chr4 | 25009191 | 25009509 | + | 0.8 | 0.026275 | 1.43 | 5.89E−05 | NM_018176 | −0.13 |
| chr4 | 25024621 | 25024749 | + | 0.78 | 0.14308 | 3.27 | 4.18E−05 | NM_018176 | −0.13 |
| chr5 | 73147145 | 73147508 | + | 0.87 | 0.110781 | 1.41 | 211E−05 | NM_001244364 | −0.13 |
| chr5 | 73469496 | 73469771 | + | −0.3 | 0.29184 | 2.98 | 1.96E−08 | NM_001244364 | −0.13 |
| chr6 | 1.12E+08 | 1.12E+08 | + | −0.28 | 0.278449 | 1.68 | 4.29E−06 | NM_001164281 | −0.13 |
| chr1 | 2.29E+08 | 2.29E+08 | + | 0.23 | 0.391248 | 1.65 | 9.30E−05 | NM_004578 | −0.14 |
| chr17 | 71433312 | 71433502 | + | 1.04 | 0.156144 | 3.12 | 6.79E−08 | NM_012121 | −0.14 |
| chr17 | 71461219 | 71462729 | + | 2.71 | 4.82E−22 | 1.31 | 4.19E−20 | NM_012121 | −0.14 |
| chr21 | 45725078 | 45725852 | + | 1.06 | 0.000573 | 1.52 | 9.05E−09 | NM_002626 | −0.14 |
| chr7 | 21657845 | 21657986 | + | 0.46 | 0.286689 | 3.54 | 1.28E−08 | NM_001277115 | −0.14 |
| chr9 | 1.08E+08 | 1.08E+08 | + | −0.54 | 0.22977 | 1.95 | 2.71E−05 | NM_080546 | −0.14 |
| chr9 | 1.08E+08 | 1.08E+08 | + | 1.46 | 0.222672 | 2.52 | 7.56E−05 | NM_080546 | −0.14 |
| chr11 | 1.06E+08 | 1.06E+08 | + | 0.94 | 0.113163 | 2.52 | 7.56E−05 | NM_032424 | −0.15 |
| chr2 | 1.23E+08 | 1.23E+08 | + | 0 | 0.593079 | 2.19 | 3.14E−07 | NR_048556 | −0.15 |
| chr8 | 5879386 | 5879562 | + | −0.13 | 0.422029 | 2.01 | 4.17E−05 | NR_040040 | −0.15 |
| chr9 | 1.31E+08 | 1.31E+08 | + | 1.04 | 0.218994 | 2.78 | 5.42E−06 | NM_001006641 | −0.15 |
| chr1 | 2.35E+08 | 2.35E+08 | + | 1.04 | 0.325105 | 2.2 | 5.95E−05 | NM_001012985 | −0.16 |
| chr11 | 1.17E+08 | 1.17E+08 | + | 0.16 | 0.427697 | 1.39 | 4.86E−05 | NM_014956 | −0.16 |
| chr8 | 24019532 | 24019740 | + | −0.59 | 0.05845 | 1.7 | 6.77E−06 | NM_001142730 | −0.16 |
| chr18 | 24113494 | 24113630 | + | 0.56 | 0.216906 | 2.42 | 5.02E−06 | NM_001142730 | −0.16 |
| chr19 | 17594685 | 17594813 | + | 1.78 | 0.001315 | 3.05 | 6.68E−08 | NM_198580 | −0.16 |
| chr5 | 52391142 | 52391595 | + | 1.01 | 0.005043 | 2.4 | 3.84E−11 | NM_004531 | −0.16 |
| chr9 | 1.34E+08 | 1.34E+08 | + | −1.35 | 0.06335 | 1.95 | 2.71E−05 | NM_006059 | −0.16 |
| chr14 | 1.06E+08 | 1.06E+08 | + | 0.13 | 0.464479 | 1.44 | 1.46E−05 | NM_145685 | −0.17 |
| chr6 | 37830297 | 37830470 | + | 1.26 | 0.156387 | 2.46 | 1.94E−05 | NM_021943 | −0.17 |
| chr13 | 24232830 | 24232958 | + | 0.74 | 0.137564 | 3.15 | 2.19E−06 | NM_001204458 | −0.18 |
| chr2 | 1.14E+08 | 1.14E+08 | + | −0.44 | 0.196391 | 1.38 | 7.74E−05 | NM_003466 | −0.18 |
| chr2 | 1.14E+08 | 1.14E+08 | + | 0.7 | 0.016184 | 1.99 | 3.68E−08 | NM_003466 | −0.18 |
| chr2 | 2.39E+08 | 2.39E+08 | + | −0.03 | 0.460616 | 1.76 | 5.85E−08 | NM_001137551 | −0.18 |
| chr20 | 55826453 | 55826700 | + | 2.54 | 0.00302 | 1.4 | 4.32E−06 | NM_001719 | −0.18 |
| chr21 | 46511547 | 46511770 | + | −0.54 | 0.3033 | 2.03 | 1.19E−06 | NR_027674 | −0.19 |
| chr22 | 50732336 | 50733389 | + | 1.94 | 9.77E−14 | 1.66 | 6.00E−15 | NM_012401 | −0.19 |
| chr22 | 50734451 | 50734594 | + | 4.04 | 2.38E−05 | 2.52 | 7.56E−05 | NM_012401 | −0.19 |
| chr6 | 50797659 | 50797966 | + | 0.19 | 0.465306 | 1.39 | 3.05E−05 | NM_003221 | −0.19 |
| chr7 | 95526342 | 95526471 | + | 0.64 | 0.009079 | 4.34 | 3.77E−06 | NM_004411 | −0.19 |
| chr9 | 1.37E+08 | 1.37E+08 | + | 1.04 | 0.325105 | 4.27 | 6.94E−06 | NM_001278074 | −0.19 |
| chr9 | 1.37E+08 | 1.37E+08 | + | −0.96 | 0.104912 | 1.43 | 2.13E−07 | NM_001278074 | −0.19 |
| chr9 | 1.37E+08 | 1.37E+08 | + | 0.23 | 0.02581 | 3.2 | 1.25E−06 | NM_001278074 | −0.19 |
| chr1 | 1.55E+08 | 1.55E+08 | + | 1.04 | 0.085353 | 2.31 | 8.78E−05 | NM_001278230 | −0.2 |
| chr18 | 47019391 | 47019580 | + | −0.44 | 0.196391 | 1.51 | 1.88E−05 | NM_000985 | −0.2 |
| chr19 | 3548581 | 3548873 | + | 0.09 | 0.430019 | 1.85 | 2.21E−07 | NM_021731 | −0.2 |
| chr3 | 48750631 | 48750763 | + | −1 | 0.064264 | 2.15 | 9.54E−05 | NM_001146179 | −0.2 |
| chr3 | 50163676 | 50163877 | + | −0.96 | 0.104912 | 2.11 | 3.97E−05 | NR_045388 | −0.2 |
| chr4 | 1.19E+08 | 1.19E+08 | + | −0.1 | 0.415961 | 1.91 | 1.75E−05 | NM_003619 | −0.2 |
| chr4 | 1.19E+08 | 1.19E+08 | + | 0.19 | 0.465306 | 3.4 | 1.33E−05 | NM_003619 | −0.2 |
| chr6 | 18198227 | 18198382 | + | 0.13 | 0.464479 | 2.46 | 1.94E−05 | NM_153042 | −0.2 |
| chr3 | 4566203 | 4566555 | + | 0.68 | 0.126482 | 1.31 | 5.07E−05 | NM_001099952 | −0.21 |
| chr3 | 1.44E+08 | 1.44E+08 | + | −0.03 | 0.460616 | 1.79 | 7.19E−06 | NM_173653 | −0.21 |
| chr3 | 1.44E+08 | 1.44E+08 | + | 0.19 | 0.424653 | 1.7 | 9.76E−05 | NM_173653 | −0.21 |
| chr4 | 36028221 | 36028436 | + | −0.04 | 0.956481 | 3.09 | 1.18E−07 | NM_015230 | −0.21 |
| chr8 | 17577806 | 17577999 | + | 1.04 | 0.085353 | 1.88 | 2.74E−05 | NM_001001931 | −0.21 |
| chr12 | 29696494 | 29696805 | + | 1.78 | 0.149771 | 1.7 | 2.56E−05 | NM_183378 | −0.22 |
| chr12 | 75867690 | 75868349 | + | 1.16 | 0.08465 | 1.96 | 4.92E−10 | NM_006851 | −0.22 |
| chr18 | 13956850 | 13957098 | + | 2.3 | 4.68E−05 | 1.34 | 2.25E−05 | NM_000529 | −0.22 |
| chr11 | 3066126 | 3066322 | + | 0.19 | 0.396995 | 2.07 | 6.37E−05 | NM_001014437 | −0.23 |
| chr13 | 28198440 | 28198931 | + | 3.46 | 0.0011 | 1.41 | 2.11E−05 | NM_152705 | −0.23 |
| chr2 | 1.59E+08 | 1.59E+08 | + | 2.92 | 0.000339 | 1.84 | 4.25E−01 | NM_001111067 | −0.23 |
| chr1 | 1.66E+08 | 1.66E+08 | + | −2.36 | 0.648257 | 2.83 | 3.17E−06 | NM_001017961 | −0.24 |
| chr1 | 1.66E+08 | 1.66E+08 | + | 0.65 | 0.173721 | 1.48 | 1.74E−07 | NM_001017961 | −0.24 |
| chr10 | 1.26E+08 | 1.26E+08 | + | 0.22 | 0.351988 | 2.64 | 2.45E−06 | NM_014661 | −0.24 |
| chr17 | 17744110 | 17744557 | + | 0.05 | 0.422088 | 1.56 | 4.49E−00 | NM_001005291 | −0.24 |
| chr19 | 6063761 | 6063815 | + | −0.28 | 0.353275 | 1.91 | 1.75E−05 | NM_134433 | −0.24 |
| chr7 | 1.32E+08 | 1.32E+08 | + | −0.17 | 0.314291 | 1.42 | 3.16E−12 | NM_001105543 | −0.24 |
| chr7 | 1.32E+08 | 1.32E+08 | + | 0.73 | 0.00011 | 1.42 | 1.57E−28 | NM_001105543 | −0.24 |
| chr7 | 1.32E+08 | 1.32E+08 | + | 0.44 | 0.077846 | 1.53 | 7.95E−20 | NM_001105543 | −0.24 |
| chr7 | 1.32E+08 | 1.32E+08 | + | 2.26 | 0.065139 | 3.05 | 6.68E−06 | NM_001105\543 | −0.24 |
| chrX | 1.53E+08 | 1.53E+08 | + | 1.46 | 0.018075 | 1.7 | 9.76E−05 | NM_001105543 | −0.24 |
| chr17 | 19549682 | 19549816 | + | −0.3 | 0.29184 | 2.41 | 3.22E−05 | NM_000382 | −0.25 |
| chr5 | 1.37E+08 | 1.37E+08 | + | 0.78 | 0.301098 | 2.31 | 8.78E−05 | NM_004598 | −0.25 |
| chr5 | 1.41E+08 | 1.41E+08 | + | 0.89 | 0.028357 | 1.79 | 3.37E−07 | NM_005471 | −0.25 |
| chr9 | 734802 | 735094 | + | 1.01 | 0.003957 | 1.46 | 4.04E−05 | NM_153186 | −0.25 |
| chr10 | 88161336 | 88161490 | + | 1.46 | 0.222672 | 1.72 | 4.05E−05 | NM_017551 | −0.26 |
| chr17 | 19212355 | 19213080 | + | 0.54 | 0.009121 | 1.46 | 1.08E−09 | NR_048576 | −0.26 |
| chr3 | 64627450 | 64627632 | + | 2.11 | 1.94E−05 | 2.07 | 6.37E−05 | NR_038264 | −0.26 |
| chr3 | 64628321 | 64628647 | + | 1.96 | 0.000304 | 1.58 | 2.17E−05 | NR_038264 | −0.26 |
| chr3 | 64644888 | 64645413 | + | 1.29 | 0.006935 | 1.88 | 1.43E−07 | NR_038264 | −0.26 |
| chr4 | 54872591 | 54873820 | + | 2.33 | 6.23E−28 | 1.66 | 4.42E−13 | NR_027153 | −0.26 |
| chr4 | 54877404 | 54877676 | + | 3.02 | 2.18E−20 | 1.85 | 3.03E−06 | NR_027153 | −0.26 |
| chr4 | 54878290 | 54878448 | + | 2.31 | 5.03E−08 | 2.46 | 3.03E−06 | NR_027153 | −0.26 |
| chr12 | 1.2E+08 | 12E+08 | + | 0.78 | 0.301098 | 2.63 | 2.67E−05 | NM_001136534 | −0.27 |
| chr19 | 45608097 | 45608261 | + | 0.57 | 0.230388 | 2.06 | 6.83E−06 | NM_019121 | −0.27 |
| chr14 | 1.05E+08 | 1.05E+08 | + | 0.81 | 0.079776 | 2.25 | 3.66E−05 | NM_015656 | −0.28 |
| chr14 | 1.05E+08 | 1.05E+08 | + | 0.46 | 0.308555 | 3.4 | 1.33E−05 | NM_015656 | −0.28 |
| chr2 | 84609937 | 84610232 | + | 0.46 | 0.167227 | 2.12 | 2.95E−07 | NM_003849 | −0.28 |
| chr8 | 1.24E+08 | 1.24E+08 | + | 0.51 | 0.147807 | 1.37 | 1.14E−10 | NM_014943 | −0.28 |
| chr14 | 49440307 | 49440548 | + | −0.74 | 0.042426 | 1.44 | 1.46E−05 | NM_001030001 | −0.29 |
| chr15 | 38807024 | 38807199 | + | 0.04 | 0.529972 | 2.6 | 4.14E−06 | NM_001128602 | −0.29 |
| chr7 | 23500778 | 23500940 | + | −0.2 | 0.353044 | 1.75 | 2.65E−05 | NM_006547 | −0.29 |
| chr8 | 1.07E+08 | 1.07E+08 | + | 0.59 | 0.149458 | 2.75 | 4.74E−08 | NM_018002 | −0.29 |
| chr11 | 1.13E+08 | 1.13E+08 | + | 0.92 | 0.152933 | 1.8 | 6.58E−05 | NR_034101 | −0.3 |
| chr14 | 72192384 | 72192771 | + | 0.37 | 0.15055 | 1.34 | 1.42E−05 | NM_015556 | −0.3 |
| chr13 | 61330417 | 61330978 | + | 0.36 | 0.303275 | 1.44 | 3.67E−07 | NM_001146070 | −0.32 |
| chr19 | 13215124 | 13215269 | + | −0.16 | 0.376068 | 2.35 | 2.16E−06 | NM_005583 | −0.32 |
| chr2 | 68642008 | 68642167 | + | −0.54 | 0.361994 | 3.05 | 6.68E−06 | NM_002664 | −0.32 |
| chr4 | 54153510 | 54153789 | + | 1.6 | 0.002197 | 2.95 | 3.39E−08 | NM_152540 | −0.32 |
| chr4 | 1.1E+08 | 1.1E+08 | + | −2.13 | 0.027712 | 2.58 | 4.50E−05 | NM_001256074 | −0.32 |
| chr4 | 1.43E+08 | 1.43E+08 | + | 2.16 | 0.016043 | 1.65 | 2.44E−05 | NM_003866 | −0.32 |
| chr4 | 1.43E+08 | 1.43E+08 | + | −0.2 | 0.32972 | 1.68 | 1.62E−05 | NM_003866 | −0.32 |
| chr4 | 1.44E+08 | 1.44E+08 | + | −0.13 | 0.422029 | 1.37 | 2.50E−06 | NM_003866 | −0.32 |
| chr5 | 1.39E+08 | 1.39E+08 | + | −1.36 | 0.791388 | 1.95 | 1.12E−05 | NM_032289 | −0.32 |
| chr10 | 52336134 | 52336787 | + | 0.78 | 0.14308 | 1.3 | 3.09E−07 | NM_147156 | −0.33 |
| chr16 | 4441454 | 4441968 | + | 1.2 | 0.002108 | 2.11 | 2.65E−10 | NM_138440 | −0.34 |
| chr2 | 74112768 | 74113003 | + | 0.29 | 0.337406 | 1.59 | 5.50E−05 | NM_001199893 | −0.35 |
| chr5 | 52159831 | 52160154 | + | 0.33 | 0.277101 | 2.32 | 3.28E−12 | NM_181501 | −0.35 |
| chr5 | 52164262 | 52164394 | + | 0.04 | 0.513686 | 3.12 | 6.79E−08 | NM_181501 | −0.35 |
| chr1 | 1.75E+08 | 1.75E+08 | + | 1.46 | 0.109973 | 2.34 | 5.64E−07 | NM_003285 | −0.36 |
| chr22 | 40795782 | 40795944 | + | 0.85 | 0.021439 | 2.05 | 2.62E−05 | NM_015705 | −0.36 |
| chr3 | 62234917 | 62235103 | + | −0.13 | 0.422029 | 2.68 | 1.57E−05 | NR_038283 | −0.38 |
| chr10 | 60726012 | 60726214 | + | −0.06 | 0.44608 | 2.73 | 9.25E−06 | NM_032439 | −0.41 |
| chr10 | 60885594 | 60885936 | + | 0.45 | 0.015307 | 1.78 | 8.03E−07 | NM_032439 | −0.41 |
| chr13 | 41172002 | 41172134 | + | −2.36 | 0.648257 | 3.73 | 4.01E−07 | NM_002015 | −0.42 |
| chr3 | 13142294 | 13142539 | + | 0.33 | 0.277101 | 1.59 | 5.50E−05 | NM_001134382 | −0.42 |
| chr1 | 15359923 | 15360135 | + | −0.96 | 0.18457 | 1.46 | 6.44E−05 | NM_001017999 | −0.43 |
| chr2 | 1.12E+08 | 1.12E+08 | + | 0.13 | 0.429293 | 1.68 | 6.15E−05 | NR_024373 | −0.44 |
| chr8 | 17626529 | 17626682 | + | 2.04 | 0.099329 | 2.6 | 4.14E−06 | NM_001001924 | −0.45 |
| chr4 | 93021108 | 93021236 | + | 2.04 | 0.099329 | 3.34 | 2.36E−05 | NM_001510 | −0.46 |
| chr4 | 93255689 | 93255904 | + | 0.45 | 0.015307 | 3.5 | 2.29E−08 | NM_001510 | −0.46 |
| chr13 | 39210948 | 39211076 | + | −2.13 | 0.027712 | 3.52 | 4.18E−06 | NM_207361 | −0.46 |
| chr5 | 1.46E+08 | 1.46E+08 | + | 1.2 | 0.002108 | 1.86 | 2.32E−13 | NM_181675 | −0.49 |
| chr5 | 1.46E+08 | 1.46E+08 | + | 1.78 | 0.149771 | 2.61 | 3.88E−07 | NM_181675 | −0.49 |
| chr5 | 1.46E+08 | 1.46E+08 | + | 2.54 | 0.00302 | 2.22 | 1.17E−08 | NM_181675 | −0.49 |
| chr5 | 1.46E+08 | 1.46E+08 | + | 1.55 | 0.000234 | 1.9 | 1.63E−08 | NM_181675 | −0.49 |
| chr5 | 1.46E+08 | 1.46E+08 | + | 0.78 | 0.201565 | 1.51 | 1.88E−05 | NM_181675 | −0.49 |
| chr5 | 1.46E+08 | 1.46E+08 | + | 2.63 | 0.027294 | 3.15 | 2.19E−06 | NM_181675 | −0.49 |
| chr8 | 64478362 | 64478992 | + | 2.49 | 7.99E−07 | 1.75 | 1.18E−09 | NR_038875 | −0.49 |
| chr2 | 1.9E+08 | 1.9E+08 | + | 0.78 | 0.301098 | 2.15 | 9.64E−05 | NM_014585 | −0.5 |
| chr11 | 1.32E+09 | 1.32E+08 | + | 1.29 | 5.83E−05 | 1.72 | 8.19E−18 | NM_018522 | −0.51 |
| chr11 | 1.32E+08 | 1.32E+08 | + | −0.24 | 0.279739 | 2.58 | 4.50E−05 | NM_016522 | −0.51 |
| chr11 | 44584566 | 44584885 | + | −0.98 | 0.015887 | 1.72 | 1.19E−06 | NM_002231 | −0.52 |
| chr14 | 1.03E+08 | 1.03E+08 | + | 0.11 | 0.462988 | 2.25 | 3.66E−05 | NR_073542 | −0.53 |
| chr1 | 31221034 | 31222083 | + | 1.57 | 1.65E−06 | 1.63 | 9.96E−19 | NM_006762 | −0.55 |
| chr1 | 31277302 | 31277909 | + | −0.78 | 0.010359 | 1.37 | 2.17E−08 | NM_006762 | −0.55 |
| chr10 | 62052666 | 62053455 | + | −0.98 | 0.010199 | 1.48 | 4.43E−08 | NM_020987 | −0.55 |
| chr10 | 62288823 | 62288970 | + | 0.98 | 0.049001 | 2.41 | 3.22E−05 | NM_001204404 | −0.55 |
| chr8 | 93065927 | 93066564 | + | 1.7 | 0.01132 | 2.04 | 1.68E−09 | NM_004349 | −0.55 |
| chr10 | 61099144 | 61099392 | + | 1.19 | 0.036046 | 2.61 | 5.72E−10 | NM_001166698 | −0.56 |
| chr8 | 93146827 | 93146961 | + | 0.04 | 0.529972 | 2.78 | 5.42E−06 | NM_01198633 | −0.56 |
| chr18 | 74792841 | 74792969 | + | −0.13 | 0.422029 | 2.73 | 9.25E−06 | NM_001025100 | −0.58 |
| chr3 | 30375463 | 30375667 | + | −0.54 | 0.3033 | 2.35 | 2.16E−06 | NM_001024847 | −0.58 |
| chr3 | 30683740 | 30683988 | + | 1.26 | 0.961907 | 1.99 | 1.72E−05 | NM_001024847 | −0.58 |
| chr3 | 30703324 | 30703592 | + | −0.06 | 0.442247 | 1.88 | 1.13E−05 | NM_001024847 | −0.58 |
| chr4 | 1.41E+08 | 1.41E+08 | + | −1.13 | 0.188905 | 2.52 | 7.56E−05 | NM_001204367 | −0.58 |
| chr5 | 62424424 | 62424557 | + | −0.36 | 0.926622 | 3.63 | 1.30E−06 | NM_181506 | −0.58 |
| chr5 | 62507265 | 62507441 | + | 2.26 | 0.065139 | 3.42 | 7.25E−08 | NM_181506 | −0.58 |
| chr6 | 1.32E+08 | 1.32E+08 | + | 0.46 | 0.404925 | 2.34 | 1.37E−05 | NM_001901 | −0.58 |
| chrX | 1.45E+08 | 1.45E+08 | + | 1.83 | 0.000917 | 2.15 | 1.70E−14 | NM_032539 | −0.58 |
| chrX | 1.45E+08 | 1.45E+08 | + | 2.26 | 0.010657 | 3.46 | 4.08E−08 | NM_032539 | −0.58 |
| chrX | 1.45E+08 | 1.45E+08 | + | 1.26 | 0.156387 | 3.42 | 7.25E−08 | NM_001144009 | −0.58 |
| chr1 | 1.87E+08 | 1.87E+08 | + | 0.46 | 0.404925 | 2.2 | 1.53E−05 | NM_024420 | −0.59 |
| chr1 | 1.87E+08 | 1.87E+08 | + | −1.96 | 0.01731 | 2.35 | 9.04E−08 | NM_024420 | −0.59 |
| chr2 | 1.83E+08 | 1.83E+08 | + | 2.04 | 0.009329 | 2.2 | 1.53E−05 | NM_001258314 | −0.63 |
| chr22 | 41057211 | 41058340 | + | 1.5 | 9.85E−06 | 1.84 | 1.99E−17 | NM_005297 | −0.65 |
| chr22 | 41072122 | 41072268 | + | 1.54 | 0.022876 | 3.1 | 3.83E−06 | NM_005297 | −0.65 |
| chr10 | 98909413 | 98909545 | + | −1.77 | 0.032657 | 2.58 | 4.50E−05 | NM_003061 | −0.68 |
| chr16 | 66300679 | 66300853 | + | −0.54 | 0.261994 | 2.36 | 5.33E−05 | NM_001795 | −0.69 |
| chr13 | 46697061 | 46697369 | + | 2.26 | 0.010657 | 1.72 | 1.19E−06 | NM_001278541 | −0.74 |
| chr20 | 57936385 | 57936687 | + | 0.52 | 0.173082 | 1.59 | 9.83E−07 | NM_207034 | −0.74 |
| chr17 | 77021745 | 77021873 | + | −0.99 | 0.000752 | 2.2 | 5.95E−05 | NR_049769 | −0.75 |
| chr2 | 14759214 | 14759421 | + | −0.22 | 0.38435 | 2.07 | 6.37E−06 | NM_145175 | −0.75 |
| chr2 | 15050407 | 15050805 | + | 0.84 | 0.035899 | 1.54 | 2.41E−08 | NM_145175 | −0.75 |
| chr14 | 32964091 | 32964219 | + | 0.72 | 0.192023 | 2.39 | 1.31E−06 | NM_004274 | −0.84 |
| chr6 | 1.29E+08 | 1.29E+08 | + | 2.95 | 2.62E−07 | 3.12 | 2.51E−14 | NM_001135648 | −0.86 |
| chr1 | 42385966 | 42386126 | + | 0.54 | 0.204578 | 2.31 | 8.78E−05 | NR_038260 | −0.94 |
| chr16 | 81440812 | 81440979 | + | −1.13 | 0.188905 | 2.58 | 4.50E−05 | NR_039871 | −0.94 |
| chr18 | 3989314 | 3989651 | + | 0.16 | 0.264283 | 1.46 | 1.60E−07 | NR_102 [symbol] | −0.97 |
| chr11 | 92593088 | 92593216 | + | 0.23 | 0.391248 | 2.31 | 5.99E−05 | NM_005959 | −1 |
| chr13 | 1.02E+08 | 1.02E+08 | + | 0.61 | 0.157103 | 1.5 | 4.71E−07 | NR_047687 | −1 |
| chr14 | 87618888 | 87619016 | + | 0.19 | 0.465306 | 2.31 | 8.78E−05 | NR_038445 | −1 |
| chr15 | 92887156 | 92887317 | + | 1.81 | 0.834416 | 2.31 | 8.78E−05 | NM_006011 | −1 |
| chr4 | 1.89E+08 | 1.89E+08 | + | 1.76 | 1.97E−06 | 1.83 | 3.41E−07 | NR_033869 | −1 |
| chr4 | 1.89E+08 | 1.89E+08 | + | −1.36 | 0.791388 | 2.63 | 2.67E−05 | NR_033869 | −1 |
| chr4 | 1.89E+08 | 1.89E+08 | + | 0.19 | 0.424653 | 1.54 | 5.74E−05 | NR_033869 | −1 |
| chr4 | 1.89E+08 | 1.89E+08 | + | 0.84 | 0.004799 | 2.07 | 7.65E−11 | NR_033869 | −1 |
| chr5 | 71837480 | 71837925 | + | 0.07 | 0.454457 | 1.48 | 6.87E−07 | NM_152625 | −1 |
| chr6 | 35013917 | 35014257 | + | −0.35 | 0.30382 | 1.9 | 1.63E−09 | NM_001261821 | −1 |
| chr8 | 1.02E+08 | 1.02E+08 | + | −0.54 | 0.215984 | 1.51 | 1.88E−05 | NM_152628 | −1 |
| chr5 | 1.4E+08 | 1.4E+09 | + | 0.78 | 0.14308 | 1.61 | 4.19E−07 | NM_031883 | −1.05 |
| chr20 | 49712632 | 49712903 | + | 3.04 | 0.007065 | 1.37 | 2.76E−05 | NM_002237 | −1.11 |
| chr20 | 49737398 | 49738692 | + | 0.12 | 0.372164 | 1.67 | 1.18E−18 | NM_002237 | −1.11 |
| chr6 | 1.07E+08 | 1.07E+08 | + | 0.78 | 0.301098 | 3.4 | 1.23E−05 | NM_182907 | −1.12 |
| chr1 | 1.61E+08 | 1.61E+08 | + | 0.04 | 0.529972 | 3.2 | 7.38E−05 | NM_021642 | −1.13 |
| chr2 | 30253886 | 30254215 | + | 0.46 | 0.404925 | 1.37 | 1.74E−05 | NM_004304 | −1.14 |
| chr18 | 31537374 | 31537520 | + | 1.36 | 0.044821 | 2.1 | 4.28E−06 | NM_001198549 | −1.2 |
| chr6 | 1.15E+08 | 1.15E+08 | + | 1.63 | 0.07633 | 2.38 | 8.30E−06 | NM_153612 | −1.21 |
| chr11 | 17834821 | 17835242 | + | 1.19 | 0.113922 | 1.68 | 1.25E−07 | NM_001112741 | −1.22 |
| chr11 | 17895454 | 17895831 | + | 0.19 | 0.396995 | 1.38 | 7.74E−05 | NM_001112741 | −1.22 |
| chr8 | 24924756 | 24924894 | + | 1.46 | 0.003445 | 2.51 | 1.16E−05 | NM_006158 | −1.22 |
| chr3 | 1.6E+08 | 1.6E4+08 | + | −0.32 | 0.326824 | 1.53 | 5.05E−06 | NM_001168214 | −1.29 |
| chr3 | 1.6E+08 | 1.6E+08 | + | −1.28 | 0.107269 | 1.81 | 2.71E−05 | NM_001168214 | −1.29 |
| chr3 | 1.6E+08 | 1.6E+08 | + | 2.83 | 4.18E−13 | 1.56 | 4.49E−09 | NM_001168214 | −1.29 |
| chr5 | 1.5E+08 | 1.5E+08 | + | 1.55 | 0.000265 | 1.37 | 1.19E−09 | NM_001166208 | −1.3 |
| chrX | 37463206 | 37463541 | + | 1.28 | 0.061907 | 1.7 | 2.05E−07 | NM_001170331 | −1.32 |
| chr7 | 1.51E+08 | 1.51E+08 | + | 0.34 | 0.120777 | 1.32 | 1.94E−16 | NM_024429 | −1.33 |
| chr7 | 1.51E+08 | 1.51E+08 | + | −0.54 | 0.329466 | 2.15 | 9.64E−05 | NM_024429 | −1.33 |
| chr11 | 8389877 | 8390012 | + | 0.94 | 0.113163 | 2.81 | 5.99E−05 | NM_001270428 | −1.4 |
| chr17 | 10018213 | 10018475 | + | 0.26 | 0.418658 | 2.03 | 1.09E−05 | NM_201432 | −1.44 |
| chr19 | 6801588 | 6802052 | + | −1.35 | 0.004025 | 1.66 | 1.25E−07 | NM_005428 | −1.46 |
| chr4 | 96134788 | 96135552 | + | 0.72 | 0.053006 | 1.75 | 1.18E−09 | NM_001256794 | −1.46 |
| chrX | 9874995 | 9875137 | + | −0.04 | 0.452279 | 2.31 | 8.78E−05 | NM_001195081 | −1.49 |
| chr17 | 17651960 | 17652696 | + | 1.6 | 6.92E−11 | 1.59 | 2.90E−12 | NR_024007 | −1.58 |
| chr17 | 17664127 | 17664362 | + | 1.83 | 0.000917 | 2.07 | 1.98E−07 | NR_024007 | −1.58 |
| chr17 | 17669266 | 17669463 | + | 1.82 | 0.000383 | 1.72 | 4.46E−06 | NR_024007 | −1.58 |
| chrX | 1.45E+08 | 1.45E+08 | + | 1.26 | 0.026644 | 2.83 | 1.67E−14 | NM_004709 | −1.58 |
| chrX | 1.45E+08 | 1.45E+08 | + | 1.98 | 0.002571 | 1.65 | 2.44E−05 | NM_004709 | −1.58 |
| chrX | 1.45E+08 | 1.45E+08 | + | 0.46 | 0.365076 | 4.2 | 1.27E−05 | NM_004709 | −1.58 |
| chrX | 1.45E+08 | 1.45E+08 | + | 2.26 | 0.010657 | 2.68 | 1.24E−09 | NM_004709 | −1.58 |
| chrX | 1.45E+08 | 1.45E+08 | + | 1.46 | 0.010305 | 2.15 | 9.64E−05 | NM_004709 | −1.58 |
| chr11 | 12101852 | 12102125 | + | −0.39 | 0.265331 | 2.34 | 5.64E−07 | NM_014632 | −1.59 |
| chr11 | 12111840 | 12112001 | + | −0.54 | 0.261994 | 2.25 | 3.66E−05 | NM_014632 | −1.59 |
| chr3 | 1.9E+08 | 1.9E+08 | + | −0.13 | 0.411748 | 4.92 | 4.20E−09 | NM_018192 | −1.6 |
| chr5 | 57312368 | 57312870 | + | 2.81 | 8.58E−08 | 1.89 | 5.16E−10 | NM_001252226 | −1.61 |
| chr5 | 57313486 | 57314877 | + | 1.28 | 5.32E−06 | 1.48 | 3.60E−12 | NM_001252226 | −1.61 |
| chr5 | 57315615 | 57316268 | + | 2.85 | 4.58E−12 | 1.4 | 2.62E−08 | NM_001252226 | −1.61 |
| chr5 | 57418112 | 57418362 | + | 0.04 | 0.529972 | 1.8 | 6.58E−05 | NM_001252226 | −1.61 |
| chr5 | 57724924 | 57725156 | + | 1.46 | 0.109973 | 3.27 | 7.19E−09 | NM_001252226 | −1.61 |
| chr7 | 4860643 | 4860771 | + | −0.54 | 0.281197 | 1.8 | 6.58E−05 | NR_039800 | −1.69 |
| chr17 | 53802711 | 53802839 | + | 1.26 | 0.061907 | 2.2 | 5.95E−05 | NM_018286 | −1.75 |
| chr6 | 1.19E+08 | 1.19E+08 | + | −0.77 | 0.876277 | 2.31 | 8.78E−05 | NR_037662 | −1.81 |
| chr10 | 45631266 | 45631624 | + | 2.29 | 7.49E−08 | 1.36 | 1.43E−06 | NR_024472 | −2 |
| chr21 | 42274009 | 42274387 | + | −0.41 | 0.248943 | 2.25 | 7.23E−09 | NR_073202 | −2.02 |
| chr7 | 32317511 | 32317647 | + | −0.54 | 0.281197 | 2.34 | 1.37E−05 | NM_001191058 | −2.05 |
| chr1 | 1.63E+08 | 1.63E+08 | + | 0.04 | 0.529972 | 2.2 | 1.88E−08 | NM_001113380 | −2.09 |
| chr3 | 11042532 | 11042833 | + | −0.13 | 0.411748 | 1.53 | 3.19E−06 | NM_003042 | −2.09 |
| chr7 | 32097768 | 32097923 | + | 1.64 | 0.000139 | 3.08 | 3.76E−09 | NM_001191056 | −2.28 |
| chr1 | 17447421 | 17447579 | + | 0.46 | 0.463277 | 1.92 | 4.25E−05 | NM_007365 | −2.32 |
| chr10 | 1.35E+08 | 1.35E+08 | + | −0.28 | 0.310976 | 2.93 | 2.01E−05 | NM_015722 | −2.32 |
| chr20 | 57121050 | 57121600 | + | −0.2 | 0.32972 | 2.02 | 8.33E−14 | NR_034147 | −2.43 |
| chr16 | 82667638 | 82666111 | + | 0.8 | 0.001044 | 1.98 | 1.55E−08 | NM_001220491 | −2.5 |
| chr1 | 89788157 | 89788474 | + | 1.26 | 0.061907 | 1.39 | 3.05E−05 | NR_077244 | −3.32 |
| chr1 | 90870532 | 90870713 | + | −1.54 | 0.008491 | 2.1 | 4.28E−06 | NM_020063 | −3.32 |
| chr1 | 1.81E+08 | 1.81E+08 | + | −1.2 | 0.001262 | 1.54 | 1.05E−20 | NM_000721 | −3.32 |
| chr11 | 59961362 | 59961523 | + | 0.56 | 0.216906 | 2.16 | 2.47E−05 | NM_001247999 | −3.32 |
| chr11 | 1.11E+08 | 1.11E+08 | + | 0.3 | 0.381963 | 1.95 | 1.12E−05 | NM_198498 | −3.32 |
| chr11 | 1.11E+08 | 1.11E+08 | + | 0.46 | 0.404925 | 2.2 | 5.95E−05 | NM_198498 | −3.32 |
| chr11 | 1.11E+08 | 1.11E+08 | + | −2.36 | 0.648257 | 3.29 | 4.03E−07 | NM_198498 | −3.32 |
| chr11 | 1.11E+08 | 1.11E+08 | + | 2.4 | 4.44E−07 | 1.92 | 0.000104 | NM_198498 | −3.32 |
| chr16 | 55314913 | 55315050 | + | −1.19 | 0.000306 | 2.52 | 7.56E−05 | NM_024335 | −3.32 |
| chr18 | 18847557 | 18848081 | + | 1.53 | 0.01294 | 2.91 | 6.64E−17 | NM_001142966 | −3.32 |
| chr18 | 18943441 | 18943744 | + | 0.46 | 0.334119 | 2.01 | 2.08E−07 | NM_001142966 | −3.32 |
| chr18 | 52755385 | 52755665 | + | −0.2 | 0.353044 | 1.9 | 2.20E−07 | NM_025214 | −3.32 |
| chr18 | 57883581 | 57885484 | + | 1.86 | 6.07E−06 | 1.86 | 2.42E.34 | NM_005912 | −3.32 |
| chr19 | 43624425 | 43626178 | + | 2.45 | 2.82E−42 | 1.31 | 1.28E−20 | NM_031246 | −3.32 |
| chr2 | 1.16E+08 | 1.16E+08 | + | 0.63 | 0.261697 | 1.6 | 8.76E−05 | NM_001004360 | −3.32 |
| chr20 | 42567918 | 42588208 | + | −1.54 | 0.103108 | 2.54 | 2.67E−09 | NM_001098798 | −3.32 |
| chr18 | 45846339 | 45846473 | + | 1.78 | 0.149771 | 2.2 | 5.95E−05 | NM_001039360 | −3.49 |
| chr13 | 43467628 | 43468040 | + | 0 | 0.593079 | 1.9 | 5.25E−07 | NM_001002264 | −4.32 |
| chr2 | 76894792 | 76894947 | + | 0.04 | 0.529972 | 2.31 | 8.78E−05 | NM_001134745 | −4.32 |
| chr5 | 1.74E+08 | 1.74E+08 | + | 2.04 | 0.000496 | 1.83 | 1.23E−09 | NM_002449 | −4.32 |
| chr5 | 1.74E+08 | 1.74E+08 | + | 0.04 | 0.529972 | 2.24 | 9.42E−06 | NM_002449 | −4.32 |
| chr5 | 1.74E+08 | 1.74E+08 | + | −1.28 | 0.107269 | 2.2 | 5.95E−05 | NM_002449 | −4.32 |
| chr19 | 18875178 | 18875362 | + | 0.13 | 0.464479 | 1.49 | 4.40E−05 | NM_000095 | −4.91 |
| chr22 | 28602385 | 28602897 | + | 0.87 | 0.062557 | 1.37 | 6.27E−06 | NR_036169 | −6.32 |

[symbol]

indicates data missing or illegible when filed

### Example 9

### Effects of p300/CBP HAT Inhibitors

To investigate the effects on MGMT expression in glioblastoma (GBM) cells by the inhibition of the enhancer, GBM cells (3080) were treated with two different p300/CBP HAT inhibitors, CBP1 and CPI-329. Both of them resulted in reduced levels of H3K27ac and reduced expression of MGMT (FIGS. 14A and 14B). The reduced expression levels of MGMT after each treatment were further confirmed by RT-qPCR (FIGS. 15A and 15B).

To test the sensitivity of GBM cells to TMZ, both neurosphere formation assay and colony formation assay were employed. GBM cells (3080) treated with p300/CBP HAT inhibitors (CBP1 and CPI-329) resulted in increased sensitivity to TMZ (FIGS. 16A and 16B).

### DOCUMENTS CITED

1. Stupp, R. et al. Radiotherapy plus Concomitant and Adjuvant Temozolomide for Glioblastoma. New England Journal of Medicine 352, 987-996, doi:doi:10.1056/NEJMoa043330 (2005).
2. Kitange, G. J. et al. Induction of MGMT expression is associated with temozolomide resistance in glioblastoma xenografts. Neuro-oncology 11, 281-291, doi:10.1215/15228517-2008-090 (2009).
3. Everhard, S. et al. Identification of regions correlating MGMT promoter methylation and gene expression in glioblastomas. Neuro-oncology 11, 348-356, doi:10.1215/15228517-2009-001 (2009).
4. Moradi, M. et al. The Prognostic Value of MGMT Promoter Methylation in Glioblastoma: A Meta-analysis of Clinical Trials. Journal of cellular physiology, doi:10.1002/jcp.25896 (2017).
5. Brandes, A. A. et al. Role of MGMT Methylation Status at Time of Diagnosis and Recurrence for Patients with Glioblastoma: Clinical Implications. The oncologist, doi:10.1634/theoncologist.2016-0254 (2017).
6. Lavon, I. et al. Novel mechanism whereby nuclear factor kappaB mediates DNA damage repair through regulation of O(6)-methylguanine-DNA-methyltransferase. Cancer research 67, 8952-8959, doi:10.1158/0008-5472.CAN-O6-3820 (2007).
7. Wickstrom, M. et al. Wnt/beta-catenin pathway regulates MGMT gene expression in cancer and inhibition of Wnt signalling prevents chemoresistance. Nature communications 6, 8904, doi:10.1038/ncomms9904 (2015).
8. Bobustuc, G. C. et al. Levetiracetam enhances p53-mediated MGMT inhibition and sensitizes glioblastoma cells to temozolomide. Neuro-oncology 12, 917-927, doi:10.1093/neuonc/noq044 (2010).
9. Taspinar, M. et al. Effect of lomeguatrib-temozolomide combination on MGMT promoter methylation and expression in primary glioblastoma tumor cells. Tumour biology: the journal of the International Society for Oncodevelopmental Biology and Medicine 34, 1935-1947, doi:10.1007/s13277-013-0738-7 (2013).
10. Quinn, J. A. et al. Phase II trial of temozolomide plus o6-benzylguanine in adults with recurrent, temozolomide-resistant malignant glioma. Journal of clinical oncology: official journal of the American Society of Clinical Oncology 27, 1262-1267, doi:10.1200/jco.2008.18.8417 (2009).
11. Blumenthal, D. T. et al. A Phase III study of radiation therapy (RT) and O(6)-benzylguanine+BCNU versus RT and BCNU alone and methylation status in newly diagnosed glioblastoma and gliosarcoma: Southwest Oncology Group (SWOG) study S0001. International journal of clinical oncology 20, 650-658, doi:10.1007/s10147-014-0769-0 (2015).
12. Warren, K. E. et al. A phase II study of O6-benzylguanine and temozolomide in pediatric patients with recurrent or progressive high-grade gliomas and brainstem gliomas: a Pediatric Brain Tumor Consortium study. Journal of neuro-oncology 106, 643-649, doi:10.1007/511060-011-0709-z (2012).
13. Park, C. K. et al. The Changes in MGMT Promoter Methylation Status in Initial and Recurrent Glioblastomas. Translational oncology 5, 393-397 (2012).
14. Kreth, S. et al. O-methylguanine-DNA methyltransferase (MGMT) mRNA expression predicts outcome in malignant glioma independent of MGMT promoter methylation. PloS one 6, e17156, doi:10.1371/journal.pone.0017156 (2011).
15. Hegi, M. E. et al. MGMT gene silencing and benefit from temozolomide in glioblastoma. The New England journal of medicine 352, 997-1003, doi:10.1056/NEJMoa043331 (2005).
16. Xie, L., Weichel, B., Ohm, J. E. & Zhang, K. An integrative analysis of DNA methylation and RNA-Seq data for human heart, kidney and liver. BMC Syst Biol 5 Suppl 3, S4, doi:10.1186/1752-0509-5-S3-S4 (2011).
17. Lund, R. J. et al. DNA methylation and Transcriptome Changes Associated with Cisplatin Resistance in Ovarian Cancer. Scientific reports 7, 1469, doi:10.1038/541598-017-01624-4 (2017).
18. Bannister, A. J. & Kouzarides, T. Regulation of chromatin by histone modifications. Cell research 21, 381-395, doi:10.1038/cr.2011.22 (2011).
19. Banerji, J., Rusconi, S. & Schaffner, W. Expression of a beta-globin gene is enhanced by remote SV40 DNA sequences. Cell 27, 299-308 (1981).
20. Shlyueva, D., Stampfel, G. & Stark, A. Transcriptional enhancers: from properties to genome-wide predictions. Nature reviews. Genetics 15, 272-286, doi:10.1038/nrg3682 (2014).
21. Heinz, S., Romanoski, C. E., Benner, C. & Glass, C. K. The selection and function of cell type-specific enhancers. Nat Rev Mol Cell Biol 16, 144-154, doi:10.1038/nrm3949 (2015).
22. Schlotter, C. M., Tietze, L., Vogt, U., Heinsen, C. V. & Hahn, A. Ki67 and lymphocytes in the pretherapeutic core biopsy of primary invasive breast cancer: positive markers of therapy response prediction and superior survival. Hormone molecular biology and clinical investigation 32, doi:10.1515/hmbci-2017-0022 (2017).
23. Zhou, Y. et al. Ki67 is a biological marker of malignant risk of gastrointestinal stromal tumors: A systematic review and meta-analysis. Medicine 96, e7911, doi: 10.1097/md.0000000000007911 (2017).
24. Kloppel, G. & La Rosa, S. Ki67 labeling index: assessment and prognostic role in gastroenteropancreatic neuroendocrine neoplasms. Virchows Archiv: an international journal of pathology, doi:10.1007/s00428-017-2258-0 (2017).
25. Kitange, G. J. et al. Inhibition of histone deacetylation potentiates the evolution of acquired temozolomide resistance linked to MGMT upregulation in glioblastoma xenografts. Clinical cancer research: an official journal of the American Association for Cancer Research 18, 4070-4079, doi: 10.1158/1078-0432.ccr-12-0560 (2012).
26. Allis, C. D. & Jenuwein, T. The molecular hallmarks of epigenetic control. Nature reviews. Genetics 17, 487-500, doi:10.1038/nrg.2016.59 (2016).
27. Braig, M. et al. Oncogene-induced senescence as an initial barrier in lymphoma development. Nature 436, 660-665, doi:10.1038/nature03841 (2005).
28. Gerson, S. L. MGMT: its role in cancer aetiology and cancer therapeutics. Nat Rev Cancer 4, 296-307, doi:10.1038/nrc1319 (2004).
29. Sur, I. & Taipale, J. The role of enhancers in cancer. Nat Rev Cancer 16, 483-493, doi:10.1038/nrc.2016.62 (2016).
30. Hnisz, D. et al. Super-enhancers in the control of cell identity and disease. Cell 155, 934-947, doi:10.1016/j.cell.2013.09.053 (2013).
31. Herz, H. M., Hu, D. & Shilatifard, A. Enhancer malfunction in cancer. Molecular cell 53, 859-866, doi:10.1016/j.molcel.2014.02.033 (2014).
32. Hilton, I. B. et al. Epigenome editing by a CRISPR-Cas9-based acetyltransferase activates genes from promoters and enhancers. Nature biotechnology 33, 510-517, doi:10.1038/nbt.3199 (2015).
33. Konermann, S. et al. Genome-scale transcriptional activation by an engineered CRISPR-Cas9 complex. Nature 517, 583-588, doi:10.1038/nature14136 (2015).
34. Ran, F. A. et al. Genome engineering using the CRISPR-Cas9 system. Nat Protoc 8, 2281-2308, doi:10.1038/nprot.2013.143 (2013).
35. Watts, G. S. et al. Methylation of discrete regions of the O6-methylguanine DNA methyltransferase (MGMT) CpG island is associated with heterochromatinization of the MGMT transcription start site and silencing of the gene. Molecular and cellular biology 17, 5612-5619 (1997).
36. Wang, Y. et al. Correlation between DNA methylation and expression of O6-methylguanine-DNA methyltransferase gene in cultured human tumor cells. Mutation research 273, 221-230 (1992).
37. EZH2 Inhibition May Prevent Chemoresistance in SCLC. Cancer discovery 7, 348, doi:10.1158/2159-8290.cd-rw2017-038 (2017).
38. Meisenberg, C. et al. Epigenetic changes in histone acetylation underpin resistance to the topoisomerase I inhibitor irinotecan. Nucleic acids research 45, 1159-1176, doi:10.1093/nar/gkw1026 (2017).
39. Muller, S., Filippakopoulos, P. & Knapp, S. Bromodomains as therapeutic targets. Expert reviews in molecular medicine 13, e29, doi:10.1017/s1462399411001992 (2011).
40. Wadhwa, E. & Nicolaides, T. Bromodomain Inhibitor Review: Bromodomain and Extra-terminal Family Protein Inhibitors as a Potential New Therapy in Central Nervous System Tumors. Cureus 8, e620, doi:10.7759/cureus.620 (2016).
41. Milite, C. et al. A novel cell-permeable, selective, and noncompetitive inhibitor of KAT3 histone acetyltransferases from a combined molecular pruning/classical isosterism approach. Journal of medicinal chemistry 58, 2779-2798, doi:10.1021/jm5019687 (2015).
42. Fang, D. et al. The histone H3.3K36M mutation reprograms the epigenome of chondroblastomas. Science (New York, N.Y.) 352, 1344-1348, doi: 10.1126/science.aae0065 (2016).
43. Kitange, G. J. et al. Retinoblastoma Binding Protein 4 Modulates Temozolomide Sensitivity in Glioblastoma by Regulating DNA Repair Proteins. Cell reports 14, 2587-2598, doi:10.1016/j.celrep.2016.02.045 (2016).
44. Zhang, H. et al. RPA Interacts with HIRA and Regulates H3.3 Deposition at Gene Regulatory Elements in Mammalian Cells. Molecular cell 65, 272-284, doi:10.1016/j.molcel.2016.11.030 (2017).
45. Hagege, H. et al. Quantitative analysis of chromosome conformation capture assays (3C-qPCR). Nat. Protocols 2, 1722-1733 (2007).
46. Sanjana, N. E., Shalem, O. & Zhang, F. Improved vectors and genome-wide libraries for CRISPR screening. Nature methods 11, 783-784, doi:10.1038/nmeth.3047 (2014).

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method for treating or ameliorating the effects of a cancer in a subject, comprising administering to the subject a therapeutically effective amount of a first agent that modulates the activity of an enhancer and a therapeutically effective amount of a second agent that is used to treat the cancer.

2. The method of claim 1, wherein the enhancer regulates the expression of O₆-methylguanine-DNA-methyltransferase (MGMT) and/or Ki67 in the subject.

3. The method of claim 1, wherein the first agent is selected from a Bromodomain inhibitor (BETi), a histone acetyltransferase inhibitor (HATi), and combinations thereof.

4. The method of claim 3, wherein the Bromodomain inhibitor (BETi) is selected from the group consisting of JQ1, I-BET151/762, PF-1, RVX-208, BMS-986158, OTX015, PLX-51107, GSK525762, INCB054329, TEN-010, GSK2820151, BAY 1238097, and combinations thereof.

5. The method of claim 3, wherein the histone acetyltransferase inhibitor (HATi) is selected from the group consisting of C646, NU-9056, PU141, EML425, L002, MB-3, CPTH2, Plumbagin, Embelin, EGCG, Curcumin, HAT inhibitor II, Garcinol, Anacardic acid, MG 149, Gossypol, CTK7A, Windorphen, LoCAM, TH1834, CTx-1, Lys-CoA, and combinations thereof.

6. The method of claim 3, wherein the histone acetyltransferase inhibitor (HATi) is a p300 HAT inhibitor.

7. The method of claim 1, wherein the second agent is temozolomide (TMZ).

8. The method of claim 7, wherein the subject is TMZ-resistant.

9. The method of claim 1, wherein the subject is a mammal.

10. The method of claim 9, wherein the mammal is a human.

11. The method of claim 1, wherein the cancer is selected from breast cancer, lung cancer, and brain tumor.

12. The method of claim 11, wherein the brain tumor is gliobastoma (GBM).

13. The method of claim 1, further comprising administering to the subject a therapeutically effective amount of a histone deaceylase inhibitor (HDACi) that is selected from the group consisting of Trichostatin A, SAHA, Belinostat, Panabiostat, Givinostat, Resminostat, Abexinostat, Quisinostat, Rocilinostat, Practinostat, CHR-3996, Valproic acid, Butyric acid, Phenylbutyric acid, Entinostat, Tacedinaline, 4SC202, Mocetinostat, Rom idepsin, Nicotinamide, Sirtinol, Cambinol, EX-527, and combinations thereof.

14. A method for delaying the emergence of temozolomide (TMZ) resistance and/or increasing the TMZ sensitivity in a subject, comprising administering to the subject a therapeutically effective amount of an agent that modulates the activity of an enhancer.

15. The method of claim 14, wherein the enhancer regulates the expression of O₆-methylguanine-DNA-methyltransferase (MGMT) in the subject.

16. The method of claim 14, wherein the agent is selected from a Bromodomain inhibitor (BETi), a histone acetyltransferase inhibitor (HATi), and combinations thereof.

17. The method of claim 16, wherein the Bromodomain inhibitor (BETi) is selected from the group consisting of JQ1, I-BET151/762, PF-1, RVX-208, BMS-986158, OTX015, PLX-51107, GSK525762, INCB054329, TEN-010, GSK2820151, BAY 1238097, and combinations thereof.

18. The method of claim 16, wherein the histone acetyltransferase inhibitor (HATi) is selected from the group consisting of C646, NU-9056, PU141, EML425, L002, MB-3, CPTH2, Plumbagin, Embelin, EGCG, Curcumin, HAT inhibitor II, Garcinol, Anacardic acid, MG 149, Gossypol, CTK7A, Windorphen, LoCAM, TH1834, CTx-1, Lys-CoA, and combinations thereof.

19. The method of claim 16, wherein the histone acetyltransferase inhibitor (HATi) is a p300 HAT inhibitor.

20. A method for reducing tumor aggressiveness in a subject, comprising administering to the subject a therapeutically effective amount of an agent that modulates the activity of an enhancer.

21. The method of claim 20, wherein the enhancer regulates the expression of Ki67 in the subject.

22. The method of claim 20, wherein the agent is selected from a Bromodomain inhibitor (BETi), a histone acetyltransferase inhibitor (HATi), and combinations thereof.

23. The method of claim 22, wherein the Bromodomain inhibitor (BETi) is selected from the group consisting of JQ1, I-BET151/762, PF-1, RVX-208, BMS-986158, OTX015, PLX-51107, GSK525762, INCB054329, TEN-010, GSK2820151, BAY 1238097, and combinations thereof.

24. The method of claim 22, wherein the histone acetyltransferase inhibitor (HATi) is selected from the group consisting of C646, NU-9056, PU141, EML425, L002, MB-3, CPTH2, Plumbagin, Embelin, EGCG, Curcumin, HAT inhibitor II, Garcinol, Anacardic acid, MG 149, Gossypol, CTK7A, Windorphen, LoCAM, TH1834, CTx-1, Lys-CoA, and combinations thereof.

25. The method of claim 22, wherein the histone acetyltransferase inhibitor (HATi) is a p300 HAT inhibitor.

26. A pharmaceutical composition comprising 1) a therapeutically effective amount of a first agent that modulates the activity of an enhancer in a subject suffering from a cancer and 2) a therapeutically effective amount of a second agent that is used to treat the cancer.

27. The pharmaceutical composition of claim 26, wherein the enhancer regulates the expression of O₆-methylguanine-DNA-methyltransferase (MGMT) and/or Ki67 in the subject.

28. The pharmaceutical composition of claim 26, wherein the first agent is selected from a Bromodomain inhibitor (BETi), a histone acetyltransferase inhibitor (HATi), and combinations thereof.

29. The pharmaceutical composition of claim 28, wherein the Bromodomain inhibitor (BETi) is selected from the group consisting of JQ1, I-BET151/762, PF-1, RVX-208, BMS-986158, OTX015, PLX-51107, GSK525762, INCB054329, TEN-010, GSK2820151, BAY 1238097, and combinations thereof.

30. The pharmaceutical composition of claim 28, wherein the histone acetyltransferase inhibitor (HATi) is selected from the group consisting of C646, NU-9056, PU141, EML425, L002, MB-3, CPTH2, Plumbagin, Embelin, EGCG, Curcumin, HAT inhibitor II, Garcinol, Anacardic acid, MG 149, Gossypol, CTK7A, Windorphen, LoCAM, TH1834, CTx-1, Lys-CoA, and combinations thereof.

31. The pharmaceutical composition of claim 28, wherein the histone acetyltransferase inhibitor (HATi) is a p300 HAT inhibitor.

32. The pharmaceutical composition of claim 26, wherein the second agent is temozolomide (TMZ).

33. The pharmaceutical composition of claim 32, wherein the subject is TMZ-resistant.

34. The pharmaceutical composition of claim 26, wherein the subject is a mammal.

35. The pharmaceutical composition of claim 34, wherein the mammal is a human.

36. The pharmaceutical composition of claim 26, wherein the cancer is selected from breast cancer, lung cancer, and brain tumor.

37. The pharmaceutical composition of claim 36, wherein the brain tumor is gliobastoma (GBM).

38. The pharmaceutical composition of claim 26, further comprising 3) a therapeutically effective amount of a histone deaceylase inhibitor (HDACi) that is selected from the group consisting of Trichostatin A, SAHA, Belinostat, Panabiostat, Givinostat, Resminostat, Abexinostat, Quisinostat, Rocilinostat, Practinostat, CHR-3996, Valproic acid, Butyric acid, Phenylbutyric acid, Entinostat, Tacedinaline, 4SC202, Mocetinostat, Rom idepsin, Nicotinamide, Sirtinol, Cambinol, EX-527, and combinations thereof.

39. A kit comprising the pharmaceutical composition according to any one of claims 26-38, and instructions of use.

40. A method for treating or ameliorating the effects of gliobastoma (GBM) in a subject that is resistant to temozolomide (TMZ), comprising administering to the subject a therapeutically effective amount of a p300 HAT inhibitor or a Bromodomain inhibitor (BETi) and a therapeutically effective amount of TMZ.

41. A method of modulating proliferation of brain tumor cells with or without activation of a MGMT enhancer, the method comprising contacting the cells with an effective amount of a composition selected from a HAT inhibitor, a p300 inhibitor, a bromodomain inhibitor, and combinations thereof.
